(19) United States
(12) Patent Application Publication
(10) Pub. No.: US 2023/0241217 A1
(43) Pub. Date: Aug. 3, 2023

(54) HYDROGEL IRRADIATION

(71) Applicant: Ascendis Pharma A/S, Hellerup (DK)

(72) Inventors: Oliver Boris STAUCH, Palo Alto, CA (US); Sebastian STARK, Heidelberg (DE); Tobias VOIGT, Heidelberg (DE); Nicola BISEK, Heidelberg (DE)

(21) Appl. No.: 17/923,314

(22) PCT Filed: May 3, 2021
(86) PCT No.: PCT/EP2021/061544

§ 371 Date: Nov. 4, 2022

(30) Foreign Application Priority Data
May 4, 2020 (EP) 20172675.9

(51) Int. Cl. A61K 41/17; A61K 47/60; A61K 47/69

(52) U.S. Cl. CPC A61K 41/17; A61K 47/60; A61K 47/6903

(57) ABSTRACT

The present invention relates to a process for the irradiation of a water-insoluble conjugate comprising a polymer Z to which a plurality of moieties -L²-L¹-D is covalently conjugated or of a water-insoluble complex comprising a plurality of releasably and non-covalently bound drug molecules D-H or D-OH embedded in a polymer Z′, wherein the process comprises the steps of (a) providing said conjugate or complex; and (b) exposing the conjugate or complex to ionizing radiation; wherein each -L²- is independently a chemical bond or is a spacer moiety; each -L¹- is independently a linker moiety covalently and reversibly attached to -D; and each -D is independently a drug moiety.

The present invention relates to a process for the irradiation of a water-insoluble conjugate comprising a polymer Z to which a plurality of moieties -L²-L¹-D is covalently conjugated or of a water-insoluble complex comprising a plurality of releasably and non-covalently bound drug molecules D-H or D-OH embedded in a polymer Z′, wherein the process comprises the steps of (a) providing said conjugate or complex; and (b) exposing the conjugate or complex to ionizing radiation; wherein each is independently a chemical bond or is a spacer moiety; each -L¹- is independently a linker moiety covalently and reversibly attached to -D; and each -D is independently a drug moiety.

Sterility of pharmaceutical compositions intended for parenteral administration is of outmost importance to ensure patient safety. Commonly used methods of sterilization include sterile filtration or heat sterilization (e.g. autoclaving). These techniques however can only be used for liquid formulations of solutions or robust drugs, that are resistant against heat treatment, respectively. Water-insoluble drug formulations, such as particle suspensions or hydrogels cannot be filter-sterilized, and autoclaving may destroy the structure of the active pharmaceutical ingredient of a drug product, which may lead to a partial or complete loss of activity or may cause undesired side-effects. Furthermore, it may be technically challenging to start with sterile soluble starting reagents and to perform all subsequent steps in a sterile environment or by aseptic manufacturing techniques.

WO2011/051406A1 discloses methods for sterilizing biodegradable PEG-based insoluble hydrogels in a liquid formulation comprising a protective solvent using gamma irradiation. However, it may not always be possible to use a liquid formulation, or the presence of a protective solvent may be undesirable, so alternative approaches to terminally sterilize water-insoluble drugs are needed.

Thus, there is a need to overcome these shortcomings at least partially.

This objective is achieved with a process for the irradiation of a water-insoluble conjugate comprising a polymer Z to which a plurality of moieties -L²-L¹-D is covalently conjugated or of a water-insoluble complex comprising a plurality of releasably and non-covalently bound drug molecules D-H or D-OH embedded in a polymer Z′, wherein the process comprises the steps of
(a) providing said conjugate or complex; and
(b) exposing the conjugate or complex to ionizing radiation;
wherein
each -L²- is independently a chemical bond or is a spacer moiety;
each -L¹- is independently a linker moiety covalently and reversibly attached to -D; and
each -D is independently a drug moiety.

It was surprisingly found that after exposure of such conjugates and complexes to ionizing radiation, the drug molecules released from such irradiated conjugates and complexes are unmodified.

The present method may be used to terminally sterilize water-insoluble conjugates comprising a polymer to which a plurality of drug moieties is covalently conjugated or to terminally sterilize water-insoluble complexes comprising a plurality of releasably and non-covalently bound drug molecules embedded in the polymer. Such terminal sterilization is advantageous, because sterile filtration is not feasible for water-insoluble compounds and synthesizing such water-insoluble conjugates in a sterile process starting from sterilized reagents is costly and prone to contamination.

Within the present invention the terms are used having the meaning as follows.

As used herein, the term “unmodified” in connection with drug molecules released from an irradiated conjugate or complex means that when releasing drug molecules from such irradiated conjugate or complex under physiological conditions (aqueous buffer, pH 7.4, 37° C.) for a time period T, in which 10% to 20% of the total number of drug moieties or drug present in the same amount of a corresponding non-irradiated conjugate or complex are released therefrom, the number of drug molecules released from the irradiated conjugate or complex in time T is at least 50%, such as least 55%, at least 60%, at least 65%, at least 70%, at last 75%, at least 80%, at least 85% or at least 90%, and not more than 150%, such as no more than 140%, no more than 130%, no more than 120%, no more than 110% or no more than 100%, of the number of drug molecules released from the non-irradiated conjugate or complex within time T and said released drug molecules have a purity of at least 80%, such as at least 85%, at least 88%, at least 90%, at least 92%, at least 94% or at least 95%, of the purity of the drug molecules released from a corresponding non-irradiated conjugate or complex within time T. It is understood that the “corresponding non-irradiated conjugate or complex” refers to a sample of the conjugate or complex having essentially the same structure as the irradiated conjugate or complex. In certain embodiments such corresponding non-irradiated conjugate or complex is from the same batch as the irradiated conjugate or complex.

As used herein the term “continuous irradiation” refers to a mode of irradiation in which the total radiation dose is administered without interruption of the ionizing irradiation.

As used herein the term “multiple radiation exposures” refers to a mode of irradiation in which the total radiation dose is administered in two or more exposures to the radiation field, such as in two, three, four, five, six, seven, eight, nine or ten exposures, with exposures interrupted by a break, in which no irradiation occurs.

As used herein the term “small molecule drug” or “small molecule drug moiety” refers to an organic drug or drug moiety having a molecular weight of less than 1000 Da, such as less than 900 Da or less than 800 Da. Such small molecule drug may comprise one or more polymer moieties, such as for example a peptide moiety.

As used herein the term “large molecule drug” or “large molecule drug moiety” refers to an organic drug or drug moiety having a molecular weight of more than 1000 Da and does not comprise a polymer. In certain embodiments the molecular weight of such large molecule drug or moiety is no more than 6 kDa, such as no more than 5 kDa.

As used herein, the term “oligonucleotide” refers to linear single- or double-stranded RNA and DNA with preferably 2 to 1000 nucleotides or base pairs, respectively, to circular single- or double-stranded RNA and DNA with preferably 500 to 30000 nucleotides or base pairs, respectively, and any modifications thereof. Modifications may include those which provide other chemical groups that incorporate additional charge, polarizability, hydrogen bonding, electrostatic interaction, and fluxionality to the nucleic acid bases or to the nucleic acid molecule as a whole. Such modifications may include 2′-position sugar modifications, 5-position pyrimidine modifications, 8-position purine modifications, modifications at exocyclic amines, substitution of 4-thiouridines, substitution of 5-bromo or 5-iodo-uracil; backbone modifications, methylations, and unusual base-pairing combinations such as for example the isobases isocytidine and isoguanidine. Modifications may also include 3′ and 5′ modifications such as capping and change of stereochemistry. The term also includes aptamers.

The term “peptide nucleic acids” refers to organic polymers having a peptidic backbone, i.e. a backbone in which the monomers are connected to each other through peptide linkages, to which nucleobases, preferably adenine, cytosine, guanine, thymine and uracil, are attached. A preferred backbone for a peptide nucleic acid comprises N-(2-aminoethyl)-glycine.

The term “peptide” as used herein refers to a chain of at least 2 and up to and including 50 amino acid monomer moieties, which may also be referred to as “amino acid residues”, linked by peptide (amide) linkages. The term “peptide” also includes peptidomimetics, such as D-peptides, peptoids and beta-peptides and combinations thereof and combinations with amino acid monomer moieties linked by peptide linkages and covers such peptidomimetic chains with up to and including 50 monomer moieties.

As used herein, the term “protein” refers to a chain of more than 50 amino acid monomer moieties, i.e. at least 51 amino acids, which may also be referred to as “amino acid residues”, linked by peptide linkages, in which preferably no more than 12000 amino acid monomers are linked by peptide linkages, such as no more than 10000 amino acid monomer moieties, no more than 8000 amino acid monomer moieties, no more than 5000 amino acid monomer moieties or no more than 2000 amino acid monomer moieties.

As used herein, the term “water-insoluble” refers to a compound of which less than 1 g can be dissolved in one liter of water at 20° C. to form a homogeneous solution. Accordingly, the term “water-soluble” refers to a compound of which 1 g or more can be dissolved in one liter of water at 20° C. to form a homogeneous solution.

It is understood that the water-insoluble conjugates comprising a polymer Z to which a plurality of moieties -L²-L¹-D is covalently conjugated as described herein are prodrugs.

As used herein the term “prodrug” refers to a drug moiety reversibly and covalently connected to a specialized protective group through a reversible prodrug linker moiety which is a linker moiety comprising a reversible linkage with the drug moiety and wherein the specialized protective group alters or eliminates undesirable properties in the parent molecule. This also includes the enhancement of desirable properties in the drug and the suppression of undesirable properties. The specialized non-toxic protective group may also be referred to as “carrier”. A prodrug releases the reversibly and covalently bound drug moiety in the form of its corresponding drug. In other words, a prodrug is a conjugate comprising a drug moiety, which is covalently and reversibly conjugated to a carrier moiety via a reversible linker moiety, which covalent and reversible conjugation of the carrier to the reversible linker moiety is either directly or through a spacer. The reversible linker may also be referred to as “reversible prodrug linker”. Such conjugate may release the formerly conjugated drug moiety in the form of a free drug, in which case the reversible linker or reversible prodrug linker is a traceless linker.

As used herein, the term “free form” of a drug means the drug in its unmodified, pharmacologically active form. Chemical degradation, such as during manufacturing or storage, may not always be avoidable, so it is understood that a drug in its free form may also comprise pharmacologically active compounds that are the result of chemical degradation of this active form.

As used herein the term “spacer” refers to a moiety that connects at least two other moieties with each other.

As used herein, the term “reversible”, “reversibly”, “degradable” or “degradably” with regard to the attachment of a first moiety to a second moiety means that the linkage that connects said first and second moiety is cleavable under physiological conditions, which physiological conditions are aqueous buffer at pH 7.4 and 37° C., with a half-life ranging from one day to three month, such as from one day to two months, such as from one day to one month. Such cleavage is non-enzymatically. Accordingly, the term “stable” with regard to the attachment of a first moiety to a second moiety means that the linkage that connects said first and second moiety exhibits a half-life of more than three months under physiological conditions.

As used herein, the term “reagent” means a chemical compound, which comprises at least one functional group for reaction with the functional group of another chemical compound or drug. It is understood that a drug comprising a functional group is also a reagent.

As used herein, the term “moiety” means a part of a molecule, which lacks one or more atom(s) compared to the corresponding reagent. If, for example, a reagent of the formula “H—X—H” reacts with another reagent and becomes part of the reaction product, the corresponding moiety of the reaction product has the structure “H—X—” or “—X—”, whereas each “-” indicates attachment to another moiety. Accordingly, a drug moiety, such as an antibiotic moiety, is released from a reversible linkage as a drug, such as an antibiotic drug.

It is understood that if the chemical structure of a group of atoms is provided and if this group of atoms is attached to two moieties or is interrupting a moiety, said sequence or chemical structure can be attached to the two moieties in either orientation, unless explicitly stated otherwise. For example, a moiety “—C(O)N(R¹)—” can be attached to two moieties or interrupting a moiety either as “—C(O)N(R¹)—” or as “—N(R¹)C(O)—”. Similarly, a moiety

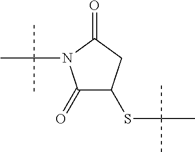

can be attached to two moieties or can interrupt a moiety either as

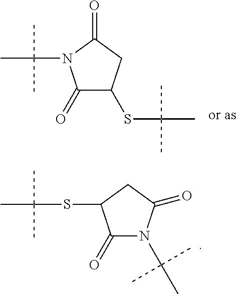

The term “substituted” as used herein means that one or more —H atoms of a molecule or moiety are replaced by a different atom or a group of atoms, which is referred to as “substituent”.

As used herein, the term “substituent” in certain embodiments refers to a moiety selected from the group consisting of halogen, —CN, —COOR^{x1}, —OR^{x1}, —C(O)R^{x1}, —C(O)N(R^{x1}R^{x1a}), —S(O)₂N(R^{x1}R^{x1a}), —S(O)N(R^{x1}R^{x1a}), —S(O)₂R^{x1}, —S(O)R^{x1}, —N(R^{x1})S(O)₂N(R^{x1a}R^{x1b}), SR^{x1}, —N(R^{x1}R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a}R^{x1b}), —OC(O)N(R^{x1}R^{x1a}), -T⁰, C₁₋₅₀ alkyl, C₂₋₅₀ alkenyl, and alkynyl; wherein -T⁰, alkyl, C₂₋₅₀ alkenyl, and C₂₋₅₀ alkynyl are optionally substituted with one or more —R^{x2}, which are the same or different and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;

—R^{x1}, —R^{x1a}, R^{x1b} are independently of each other selected from the group consisting of —H, -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R^{x2}, which are the same or different and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—; —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;

each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2}, which are the same or different;

each —R^{x2} is independently selected from the group consisting of halogen, —CN, oxo (═O), —COOR^{x4}, —OR^{x4}, —C(O)R^{x4}, —C(O)N(R^{x4}R^{x4a}), —S(O)₂N(R^{x4}R^{x4a}), —S(O)N(R^{x4}R^{x4a}), —S(O)₂R^{x4}, —S(O)R^{x4}, —N(R^{x4})S(O)₂N(R^{x4a}R^{x4b}), —SR^{x4}, —N(R^{x4}R^{x4a}), —NO₂, —OC(O)R^{x4}, —N(R^{x4})C(O)R^{x4a}, —N(R^{x4})S(O)₂R^{x4a}, —N(R^{x4})S(O)R^{x4a}, —N(R^{x4})C(O)OR^{x4a}, —N(R^{x4})C(O)N(R^{x4a}R^{x4b}), —OC(O)N(R^{x4}R^{x4a}), and C₁₋₆ alkyl; wherein C₁₋₆ alkyl is optionally substituted with one or more halogen, which are the same or different;

each —R^{x3}, —R^{x3a}, —R^{x4}, —R^{x4a}, —R^{x4b} is independently selected from the group consisting of —H and C₁₋₆ alkyl; wherein C₁₋₆ alkyl is optionally substituted with one or more halogen, which are the same or different.

In certain embodiments a maximum of 6 —H atoms of an optionally substituted molecule are independently replaced by a substituent, e.g. 5 —H atoms are independently replaced by a substituent, 4 —H atoms are independently replaced by a substituent, 3 —H atoms are independently replaced by a substituent, 2 —H atoms are independently replaced by a substituent, or 1 —H atom is replaced by a substituent.

As used herein the term “crosslinker” refers to a moiety that is a connection between different elements of a hydrogel, such as between two or more backbone moieties or between two or more hyaluronic acid strands.

As used herein, the term “hydrogel” means a hydrophilic or amphiphilic polymeric network composed of homopolymers or copolymers, which is insoluble due to the presence of hydrophobic interactions, hydrogen bonds, ionic interactions and/or covalent chemical crosslinks. The crosslinks provide the network structure and physical integrity.

As used herein the term “about” in combination with a numerical value is used to indicate a range ranging from and including the numerical value plus and minus no more than 25% of said numerical value, such as no more than plus and minus 20% of said numerical value or such as no more than plus and minus 10% of said numerical value. For example, the phrase “about 200” is used to mean a range ranging from and including 200+/−25%, i.e. ranging from and including 150 to 250; such as 200+/−20%, i.e. ranging from and including 160 to 240; such as ranging from and including 200+/−10%, i.e. ranging from and including 180 to 220. It is understood that a percentage given as “about 50%” does not mean “50%+/−25%”, i.e. ranging from and including 25 to 75%, but “about 50%” means ranging from and including 37.5 to 62.5%, i.e. plus and minus 25% of the numerical value which is 50.

As used herein, the term “polymer” means a molecule comprising repeating structural units, i.e. the monomers, connected by chemical bonds in a linear, circular, branched, crosslinked or dendrimeric way or a combination thereof, which may be of synthetic or biological origin or a combination of both. The monomers may be identical, in which case the polymer is a homopolymer, or may be different, in which case the polymer is a heteropolymer. A heteropolymer may also be referred to as a “copolymer” and includes, for example, alternating copolymers in which monomers of different types alternate, periodic copolymers, in which monomers of different types are arranged in a repeating sequence; statistical copolymers, in which monomers of different types are arranged randomly; block copolymers, in which blocks of different homopolymers consisting of only one type of monomers are linked by a covalent bond; and gradient copolymers, in which the composition of different monomers changes gradually along a polymer chain. It is understood that a polymer may also comprise one or more other moieties, such as, for example, one or more functional groups. The term “polymer” also relates to a peptide or protein, even though the side chains of individual amino acid residues may be different. It is understood that for covalently crosslinked polymers, such as hydrogels, no meaningful molecular weight ranges can be provided.

As used herein, the term “polymeric” refers to a reagent or a moiety comprising one or more polymers or polymer moieties. A polymeric reagent or moiety may optionally also comprise one or more other moieties, which in certain embodiments are selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl; and
linkages selected from the group comprising

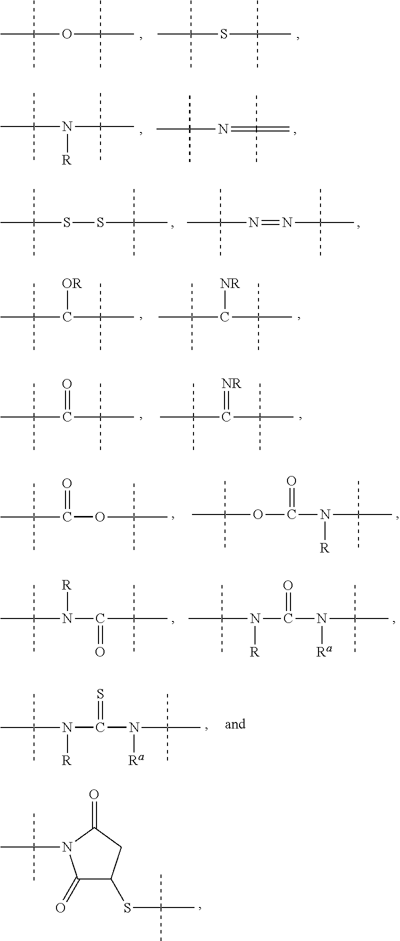

wherein
dashed lines indicate attachment to the remainder of the moiety or reagent, and
—R and —R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and which moieties and linkages are optionally further substituted.

The person skilled in the art understands that the polymerization products obtained from a polymerization reaction do not all have the same molecular weight, but rather exhibit a molecular weight distribution. Consequently, the molecular weight ranges, molecular weights, ranges of numbers of monomers in a polymer and numbers of monomers in a polymer as used herein, refer to the number average molecular weight and number average of monomers, i.e. to the arithmetic mean of the molecular weight of the polymer or polymeric moiety and the arithmetic mean of the number of monomers of the polymer or polymeric moiety.

Accordingly, in a polymeric moiety comprising “x” monomer units any integer given for “x” therefore corresponds to the arithmetic mean number of monomers. Any range of integers given for “x” provides the range of integers in which the arithmetic mean numbers of monomers lies. An integer for “x” given as “about x” means that the arithmetic mean numbers of monomers lies in a range of integers of x+/−25%, such as x+/−20% or such as x+/−10%.

As used herein, the term “number average molecular weight” means the ordinary arithmetic mean of the molecular weights of the individual polymers.

As used herein, the term “PEG-based” in relation to a moiety or reagent means that said moiety or reagent comprises PEG. Such PEG-based moiety or reagent comprises at least 10% (w/w) PEG, such as at least 20% (w/w) PEG, such as at least 30% (w/w) PEG, such as at least 40% (w/w) PEG, such as at least 50% (w/w), such as at least 60 (w/w) PEG, such as at least 70% (w/w) PEG, such as at least 80% (w/w) PEG, such as at least 90% (w/w) PEG, or such as at least 95% (w/w) PEG. The remaining weight percentage of the PEG-based moiety or reagent may be other moieties, such as those selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl; and
linkages selected from the group consisting of

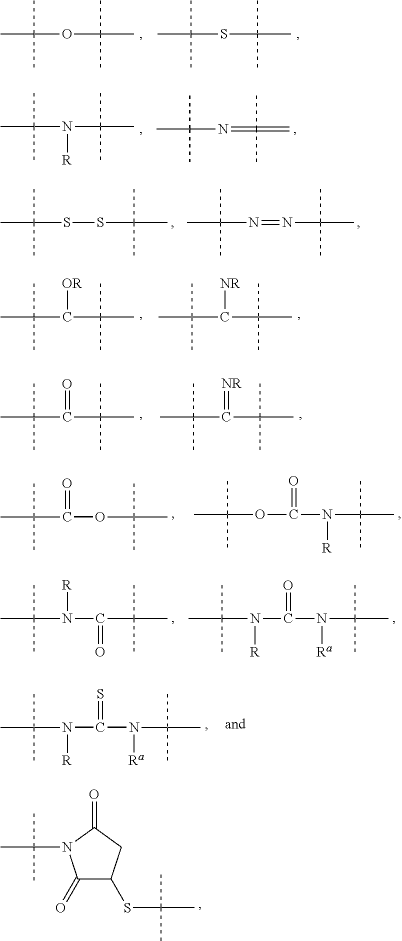

wherein
dashed lines indicate attachment to the remainder of the moiety or reagent, and
—R and —R^{a} are independently of each other selected from the group consisting of —H, and C₁₋₆ alkyl; and
which moieties and linkages are optionally further substituted.

The terms “poly(alkylene glycol)-based”, “poly(propylene glycol)-based” and “hyaluronic acid-based” are used accordingly.

The term “interrupted” means that a moiety is inserted between two carbon atoms or—if the insertion is at one of the moiety's ends—between a carbon or heteroatom and a hydrogen atom.

As used herein, the term “C₁₋₄ alkyl” alone or in combination means a straight-chain or branched alkyl moiety having 1 to 4 carbon atoms. If present at the end of a molecule, examples of straight-chain or branched C₁₋₄ alkyl are methyl, ethyl, n-propyl, isopropyl, n-butyl, isobutyl, sec-butyl and tert-butyl. When two moieties of a molecule are linked by the C₁₋₄ alkyl, then examples for such C₁₋₄ alkyl groups are —CH₂—, —CH₂—CH₂—, —CH(CH₃)—, —CH₂—CH₂—CH₂—, —CH(C₂H₅)—, —C(CH₃)₂—. Each hydrogen of a C₁₋₄ alkyl carbon may optionally be replaced by a substituent as defined above. Optionally, a C₁₋₄ alkyl may be interrupted by one or more moieties as defined below.

As used herein, the term “C₁₋₆ alkyl” alone or in combination means a straight-chain or branched alkyl moiety having 1 to 6 carbon atoms. If present at the end of a molecule, examples of straight-chain and branched C₁₋₆ alkyl groups are methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. When two moieties of a molecule are linked by the C₁₋₆ alkyl group, then examples for such C₁₋₆ alkyl groups are —CH₂—, —CH₂—CH₂—, —CH(CH₃)—, —CH₂—CH₂—CH₂—, —CH(C₂H₅)— and —C(CH₃)₂—. Each hydrogen atom of a C₁₋₆ carbon may optionally be replaced by a substituent as defined above. Optionally, a C₁₋₆ alkyl may be interrupted by one or more moieties as defined below.

Accordingly, “C₁₋₁₀ alkyl”, “C₁₋₂₀ alkyl” or “C₁₋₅₀ alkyl” means an alkyl chain having 1 to 10, 1 to 20 or 1 to 50 carbon atoms, respectively, wherein each hydrogen atom of the C₁₋₁₀, C₁₋₂₀ or C₁₋₅₀ carbon may optionally be replaced by a substituent as defined above. Optionally, a C₁₋₁₀ or C₁₋₅₀ alkyl may be interrupted by one or more moieties as defined below.

As used herein, the term “C₂₋₆ alkenyl” alone or in combination means a straight-chain or branched hydrocarbon moiety comprising at least one carbon-carbon double bond having 2 to 6 carbon atoms. If present at the end of a molecule, examples are —CH═CH₂, —CH═CH—CH₃, —CH₂—CH═CH₂, —CH═CH—CH₂—CH₃ and —CH═CH—CH═CH₂. When two moieties of a molecule are linked by the C₂₋₆ alkenyl group, then an example for such C₂₋₆ alkenyl is —CH═CH—. Each hydrogen atom of a C₂₋₆ alkenyl moiety may optionally be replaced by a substituent as defined above. Optionally, a C₂₋₆ alkenyl may be interrupted by one or more moieties as defined below.

Accordingly, the terms “C₂₋₁₀ alkenyl”, “C₂₋₂₀ alkenyl” or “C₂₋₅₀ alkenyl” alone or in combination mean a straight-chain or branched hydrocarbon moiety comprising at least one carbon-carbon double bond having 2 to 10, 2 to 20 or 2 to 50 carbon atoms, respectively. Each hydrogen atom of a C₂₋₁₀ alkenyl, C₂₋₂₀ alkenyl or C₂₋₅₀ alkenyl group may optionally be replaced by a substituent as defined above. Optionally, a C₂₋₁₀ alkenyl, C₂₋₂₀ alkenyl or C₂₋₅₀ alkenyl may be interrupted by one or more moieties as defined below.

As used herein, the term “C₂₋₆ alkynyl” alone or in combination means a straight-chain or branched hydrocarbon moiety comprising at least one carbon-carbon triple bond having 2 to 6 carbon atoms. If present at the end of a molecule, examples are —C≡CH, —CH₂—C≡CH, CH₂—CH₂—C≡CH and CH₂—C≡C—CH₃. When two moieties of a molecule are linked by the alkynyl group, then an example is —C≡C—. Each hydrogen atom of a C₂₋₆ alkynyl group may optionally be replaced by a substituent as defined above. Optionally, one or more double bond(s) may occur. Optionally, a C₂₋₆ alkynyl may be interrupted by one or more moieties as defined below.

Accordingly, as used herein, the term “C₂₋₁₀ alkynyl”, “C₂₋₂₀ alkynyl” and “C₂₋₅₀ alkynyl” alone or in combination means a straight-chain or branched hydrocarbon moiety comprising at least one carbon-carbon triple bond having 2 to 10, 2 to 20 or 2 to 50 carbon atoms, respectively. Each hydrogen atom of a C₂₋₁₀ alkynyl, C₂₋₂₀ alkynyl or C₂₋₅₀ alkynyl group may optionally be replaced by a substituent as defined above. Optionally, one or more double bond(s) may occur. Optionally, a C₂₋₁₀ alkynyl, C₂₋₂₀ alkynyl or C₂₋₅₀ alkynyl may be interrupted by one or more moieties as defined below.

As mentioned above, a C₁₋₄ alkyl, C₁₋₆ alkyl, C₁₋₁₀ alkyl, C₁₋₂₀ alkyl, C₁₋₅₀ alkyl, C₂₋₆ alkenyl, C₂₋₁₀ alkenyl, C₂₋₂₀ alkenyl, C₂₋₅₀ alkenyl, C₂₋₆ alkynyl, C₂₋₁₀ alkynyl, C₂₋₂₀ alkenyl or C₂₋₅₀ alkynyl may optionally be interrupted by one or more moieties which may be selected from the group consisting of

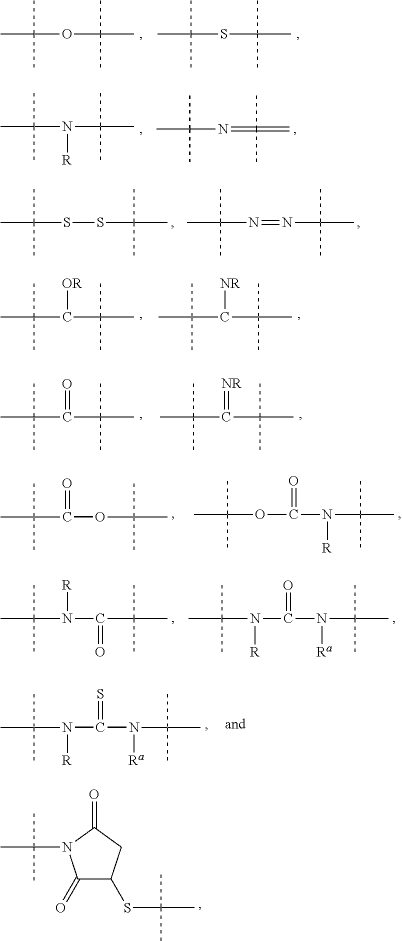

wherein
dashed lines indicate attachment to the remainder of the moiety or reagent; and
—R and —R^{a} are independently of each other selected from the group consisting of —H and C₁₋₆ alkyl.

As used herein, the term “C₃₋₁₀ cycloalkyl” means a cyclic alkyl chain having 3 to 10 carbon atoms, which may be saturated or unsaturated, e.g. cyclopropyl, cyclobutyl, cyclopentyl, cyclohexyl, cyclohexenyl, cycloheptyl, cyclooctyl, cyclononyl or cyclodecyl. Each hydrogen atom of a C₃₋₁₀ cycloalkyl carbon may be replaced by a substituent as defined above. The term “C₃₋₁₀ cycloalkyl” also includes bridged bicycles like norbornane or norbornene.

The term “8- to 30-membered carbopolycyclyl” or “8- to 30-membered carbopolycycle” means a cyclic moiety of two or more rings with 8 to 30 ring atoms, where two neighboring rings share at least one ring atom and that may contain up to the maximum number of double bonds (aromatic or non-aromatic ring which is fully, partially or un-saturated). In one embodiment an 8- to 30-membered carbopolycyclyl means a cyclic moiety of two, three, four or five rings. In another embodiment an 8- to 30-membered carbopolycyclyl means a cyclic moiety of two, three or four rings.

As used herein, the term “3- to 10-membered heterocyclyl” or “3- to 10-membered heterocycle” means a ring with 3, 4, 5, 6, 7, 8, 9 or 10 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for 3- to 10-membered heterocycles include but are not limited to aziridine, oxirane, thiirane, azirine, oxirene, thiirene, azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine and homopiperazine. Each hydrogen atom of a 3- to 10-membered heterocyclyl or 3- to 10-membered heterocyclic group may be replaced by a substituent.

As used herein, the term “8- to 11-membered heterobicyclyl” or “8- to 11-membered heterobicycle” means a heterocyclic moiety of two rings with 8 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including=N(O)—) and wherein the ring is linked to the rest of the molecule via a carbon or nitrogen atom. Examples for an 8- to 11-membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquinoline, tetrahydroisoquinoline, dihydroisoquinoline, benzazepine, purine and pteridine. The term 8- to 11-membered heterobicycle also includes spiro structures of two rings like 1,4-dioxa-8-azaspiro[4.5]decane or bridged heterocycles like 8-aza-bicyclo[3.2.1]octane. Each hydrogen atom of an 8- to 11-membered heterobicyclyl or 8- to 11-membered heterobicycle carbon may be replaced by a substituent.

Similary, the term “8- to 30-membered heteropolycyclyl” or “8- to 30-membered heteropolycycle” means a heterocyclic moiety of more than two rings with 8 to 30 ring atoms, such as of three, four or five rings, where two neighboring rings share at least one ring atom and that may contain up to the maximum number of double bonds (aromatic or non-aromatic ring which is fully, partially or unsaturated), wherein at least one ring atom up to 10 ring atoms are replaced by a heteroatom selected from the group of sulfur (including —S(O)—, —S(O)₂—), oxygen and nitrogen (including ═N(O)—) and wherein the ring is linked to the rest of a molecule via a carbon or nitrogen atom.

It is understood that the phrase “the pair R^{x}/R^{y} is joined together with the atom to which they are attached to form a C₃₋₁₀ cycloalkyl or a 3- to 10-membered heterocyclyl” in relation with a moiety of the structure

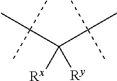

means that R^{x} and R^{y} form the following structure:

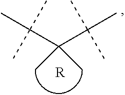

wherein R is C₃₋₁₀ cycloalkyl or 3- to 10-membered heterocyclyl.

It is also understood that the phrase “the pair R^{x}/R^{y} is joint together with the atoms to which they are attached to form a ring A” in relation with a moiety of the structure

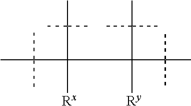

means that R^{x} and R^{y} form the following structure:

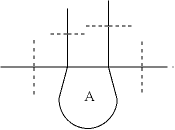

As used herein, “halogen” means fluoro, chloro, bromo or iodo. In certain embodiments halogen is fluoro or chloro.

As used herein the term “alkali metal ion” refers to Na⁺, K⁺, Li⁺, Rb⁺ and Cs⁺. In certain embodiments “alkali metal ion” refers to Na⁺, K⁺ and Li⁺.

As used herein the term “alkaline earth metal ion” refers to Mg²⁺, Ca²⁺, Sr²⁺ and Ba²⁺. In certain embodiments an alkaline earth metal ion is Mg²⁺ or Ca²⁺.

As used herein, the term “functional group” means a group of atoms which can react with other groups of atoms. Exemplary functional groups are carboxylic acid, primary amine, secondary amine, tertiary amine, maleimide, thiol, sulfonic acid, carbonate, carbamate, hydroxyl, aldehyde, ketone, hydrazine, isocyanate, isothiocyanate, phosphoric acid, phosphonic acid, haloacetyl, alkyl halide, acryloyl, aryl fluoride, hydroxylamine, disulfide, sulfonamides, sulfuric acid, vinyl sulfone, vinyl ketone, diazoalkane, oxirane, and aziridine.

In case the conjugates or complexes of the present invention comprise one or more acidic or basic groups, the invention also comprises their corresponding pharmaceutically or toxicologically acceptable salts, in particular their pharmaceutically utilizable salts. Thus, the conjugates or complexes of the present invention comprising acidic groups can be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine, amino acids, and quarternary ammonium salts, like tetrabutylammonium or cetyl trimethylammonium. Conjugates or complexes of the present invention comprising one or more basic groups, i.e. groups which can be protonated, can be present and can be used according to the invention in the form of their addition salts with inorganic or organic acids. Examples for suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, trifluoroacetic acid, and other acids known to the person skilled in the art. For the person skilled in the art further methods are known for converting the basic group into a cation like the alkylation of an amine group resulting in a positively-charge ammonium group and an appropriate counterion of the salt. If the conjugates or complexes of the present invention simultaneously comprise acidic and basic groups, the invention also includes, in addition to the salt forms mentioned, inner salts or betaines (zwitterions). The respective salts can be obtained by customary methods, which are known to the person skilled in the art like, for example by contacting these conjugates with an organic or inorganic acid or base in a solvent or dispersant, or by anion exchange or cation exchange with other salts. The present invention also includes all salts of the conjugates or complexes of the present invention which, owing to low physiological compatibility, are not directly suitable for use in pharmaceuticals but which can be used, for example, as intermediates for chemical reactions or for the preparation of pharmaceutically acceptable salts.

The term “pharmaceutically acceptable” means a substance that does not cause harm when administered to a patient and in certain embodiments means approved by a regulatory agency, such as the EMA (Europe) and/or the FDA (US) and/or any other national regulatory agency for use in animals, such as for use in humans. In certain embodiments “pharmaceutically acceptable” means Generally Recognized As Safe (GRAS) as described, for example, by the FDA in sections 201(s) and 409 of the Federal Food, Drug, and Cosmetic Act.

As used herein, the term “excipient” refers to a diluent, adjuvant, or vehicle with which the therapeutic, such as a drug or prodrug, is administered. Such pharmaceutical excipient may be sterile liquids, such as water and oils, including those of petroleum, animal, vegetable or synthetic origin, including but not limited to peanut oil, soybean oil, mineral oil, sesame oil and the like. Water is a preferred excipient when the pharmaceutical composition is administered orally. Saline and aqueous dextrose are preferred excipients when the pharmaceutical composition is administered intravenously. Saline solutions and aqueous dextrose and glycerol solutions are preferably employed as liquid excipients for injectable solutions. Suitable pharmaceutical excipients include for example starch, glucose, lactose, sucrose, mannitol, trehalose, gelatin, malt, rice, flour, chalk, silica gel, sodium stearate, glycerol monostearate, talc, sodium chloride, dried skim milk, glycerol, propylene, glycol, hyaluronic acid, propylene glycol, water, ethanol and the like. The pharmaceutical composition, if desired, can also contain minor amounts of wetting or emulsifying agents, pH buffering agents, like, for example, acetate, succinate, tris, carbonate, phosphate, HEPES (4-(2-hydroxyethyl)-1-piperazineethanesulfonic acid), MES (2-(N-morpholino)ethanesulfonic acid), or may contain detergents, like Tween, poloxamers, poloxamines, CHAPS, Igepal, or amino acids like, for example, glycine, lysine, or histidine. These pharmaceutical compositions can take the form of solutions, suspensions, emulsions, tablets, pills, capsules, powders, sustained-release formulations and the like. The pharmaceutical composition can be formulated as a suppository, with traditional binders and excipients such as triglycerides. Oral formulation can include standard excipients such as pharmaceutical grades of mannitol, lactose, starch, magnesium stearate, sodium saccharine, cellulose, magnesium carbonate, etc. Such compositions will contain a therapeutically effective amount of the drug or drug moiety, together with a suitable amount of excipient so as to provide the form for proper administration to the patient. The formulation should suit the mode of administration.

In general, the terms “comprise” or “comprising” also encompasses “consist of” or “consisting of”.

In one embodiment the process is a process for the irradiation of a water-insoluble conjugate comprising a polymer Z to which a plurality of moieties -L²-L¹-D is covalently conjugated, wherein the process comprises the steps of
(a) providing said conjugate; and
(b) exposing the conjugate to ionizing radiation; and
wherein
each -L²- is independently a chemical bond or is a spacer moiety;
each -L¹- is independently a linker moiety covalently and reversibly attached to -D; and
each -D is independently a drug moiety.

In another embodiment the process is a process for the irradiation of a water-insoluble complex comprising a plurality of releasably and non-covalently bound drug molecules D-H or D-OH embedded in a polymer Z′, wherein the process comprises the steps of
(a) providing said complex; and
(b) exposing the complex to ionizing radiation.

The ionizing radiation may be achieved with radiation selected from the group consisting of electron beam radiation, X-ray radiation, gamma radiation, proton beam radiation, neutron beam radiation, positron beam radiation, alpha particle radiation, UV radiation and any combination thereof.

In certain embodiment the ionizing radiation is electron beam radiation. In certain embodiments the ionizing radiation is X-ray radiation. In certain embodiments the ionizing radiation is gamma radiation. In certain embodiments the ionizing radiation is proton beam irradiation. In certain embodiments the ionizing radiation is neutron beam radiation. In certain embodiments the ionizing radiation is positron beam radiation. In certain embodiments the ionizing radiation is alpha particle radiation. In certain embodiments the ionizing radiation is UV radiation. In certain embodiments the ionizing radiation is a combination of 2 or more, such as 2, 3, 4, or 5, different types of radiation selected from the group consisting of X-ray radiation, gamma radiation, electron beam radiation, proton beam radiation, neutron beam radiation, positron beam radiation, alpha particle radiation, and UV radiation.

Irradiation may be performed as a continuous irradiation or as multiple irradiation exposures. Multiple exposures provide the option of cooling, incubating or storing the material to be irradiated between irradiation exposures, which may be advantageous for sensitive compounds, such as protein moieties or molecules.

The exposure to ionizing radiation in step (b) may be performed with a total absorbed radiation dose ranging from 10 to 80 kGy. In certain embodiments the total absorbed radiation dose in step (b) ranges from 12 to 60 kGy. In certain embodiments the total absorbed radiation dose in step (b) ranges from 15 to 50 kGy. In certain embodiments the total absorbed radiation dose in step (b) ranges from 17 to 40 kGy. In certain embodiments the total absorbed radiation dose in step (b) ranges from 17.5 to 35 kGy. In certain embodiment the total absorbed radiation dose in step (b) is 17.5 kGy. In certain embodiment the total absorbed radiation dose in step (b) is 25 kGy. It is understood that due to technical restrictions it may be impossible to irradiate with an exact amount of radiation, e.g. due to varying spatial distances from the radiation source. Therefore, all radiation doses provided herein refer to the nominal minimum dose, i.e. to the dose that every sample in a particular irradiation process must at least absorb. This means that certain samples may absorb a higher radiation dose. A total absorbed radiation dose of for example 25 kGy may be achieved with one continuous exposure or with multiple irradiation exposures of, for example, five exposures each at 5-kGy absorbed radiation dose.

In certain embodiments the conjugate or complex is in a liquid formulation in step (b), in particular in an aqueous formulation, such as in an aqueous buffer formulation. Such liquid formulation comprises the conjugate or complex, a liquid and optionally one or more excipients. Accordingly, the liquid is water in case of an aqueous formulation and comprises one or more buffering agents in case of an aqueous buffer formulation.

In certain embodiments the conjugate or complex is in a dry formulation in step (b). Such dry formulation may be obtained by lyophilization, vacuum drying or spray drying of a liquid formulation comprising the conjugate or complex and optionally one or more excipients. In certain embodiments the dry formulation is obtained by lyophilization. In certain embodiments the dry formulation is obtained by vacuum drying. In certain embodiments the dry formulation is obtained by spray drying.

Such liquid or dry formulation may comprise one type of conjugate or complex, i.e. the same type of conjugate or complex molecules, or may comprise more than one type of conjugate or complex, i.e. comprises a mixture of different types of conjugates or complexes. Such different types of conjugate or complex may for example vary in the type of drug covalently conjugated to the conjugate or non-covalently embedded in the complex. There may also be differences in the moiety -L¹-, either with the same moieties -D or with different moieties -D, such as to allow for a combination of different release half-lives, allowing for example for a combined fast release and a slow release of drug. Alternatively, using more than one type of -L¹- may allow for a first drug to be released with a fast release half-life and a second drug with a slow release half-life.

Step (b) may in certain embodiments be performed at a temperature ranging from −196° C. to +45° C. A temperature of −196° C. may be achieved by irradiating the conjugate or complex in a container stored in liquid nitrogen. In certain embodiments the conjugate or complex or a container comprising said conjugate or complex may be in contact with or in proximity to dry ice during step (b), such as by placing such container on or in or in proximity to the dry ice. In certain embodiments the conjugate or complex or the container comprising said conjugate or complex may be in contact with or in proximity to ice during step (b), such as by placing such containers on or in the ice. In general, it is advantageous if the conjugate or complex is cooled during step (b), because cooling reduces potential damage to sensitive drug moieties and molecules, such as protein moieties. In certain embodiments step (b) occurs at ambient temperature, i.e. at a temperature ranging from 15° C. to 45° C. It is understood that the term “ambient temperature” refers to the temperature present in the irradiation chamber.

Step (b) may also occur under vacuum or in an inert gas atmosphere which may be achieved by placing the conjugate or complex in a container with a vacuum or an inert gas atmosphere. Such inert gases may for example be selected from the group consisting of argon, helium, neon, xenon and krypton and in certain embodiments the inert gas is argon.

In certain embodiments the conjugate or complex of step (a) is a mixture of two or more different conjugates or complexes or a mixture of at least one conjugate and at least one complex. This may be achieved if the conjugate or complex is for example in the form of a particle, in which case different types of particles will be mixed.

If the conjugate or complex comprises protein moieties or protein drugs, said conjugate or complex may be in liquid or dry formulation in step (b). If the irradiation of step (b) is in the form of gamma irradiation, the conjugate or complex in certain embodiments is in a liquid formulation during step (b).

Each -D may be independently selected from the group consisting of small molecule drug moieties, large molecule drug moieties, oligonucleotide moieties, peptide nucleic acid moieties, peptide moieties and protein moieties or may comprise any combination thereof. Each -D may independently be a conjugate, such as a conjugate comprising at least one antibody or antibody fragment conjugated to one or more small molecule drug moieties, large molecule drug moieties, oligonucleotide moieties, peptide nucleic acid moieties, peptide moieties and/or protein moieties. Each -D may independently be a conjugate in which one or more small molecule drug moiety, large molecule drug moiety, oligonucleotide moiety, peptide nucleic acid moiety, peptide moiety and/or protein moiety is either stably or reversibly conjugated to one or more polymeric moiety. In certain embodiments all moieties -D of the conjugate or complex are identical and may be connected to the same or different types of -L¹-. In certain embodiments the conjugate or complex comprises more than one type of -D, such as two, three, four or five different types of -D. Such different types of -D may be connected to the same or different type of moiety -L¹-. Using different types of moiety -L¹- may for example facilitate different release half-lives for either the same or different drugs, which allows a combination of for example a fast and a slow release.

In certain embodiments -D is a small molecule drug moiety. In certain embodiments -D is a large molecule drug moiety. In certain embodiments -D is an oligonucleotide drug moiety. In certain embodiments -D is a peptide nucleic acid drug moiety. In certain embodiments -D is a peptide moiety. In certain embodiments -D is a protein moiety. In certain embodiments the conjugate or complex comprises moieties -D in the form of small molecule drug moieties and in addition comprises moieties -D in the form of peptide drug moieties. In certain embodiments the conjugate or complex comprises moieties -D in the form of small molecule drug moieties and in addition comprises moieties -D in the form of protein drug moieties. In certain embodiments the conjugate or complex comprises moieties -D in the form of peptide drug moieties and in addition comprises moieties -D in the form of protein drug moieties.

In certain embodiments -D is selected from the group consisting of consisting of central nervous system-active drug moieties, anti-infective drug moieties, anti-allergic drug moieties, immunomodulating drug moieties, anti-obesity drug moieties, anticoagulant drug moieties, antidiabetic drug moieties, anti-neoplastic drug moieties, antibacterial drug moieties, anti-viral drug moieties, anti-fungal drug moieties, analgesic drug moieties, contraceptive drug moieties, anti-inflammatory drug moieties, steroidal drug moieties, vasodilating drug moieties, vasoconstricting drug moieties, and cardiovascular drug moieties.

In certain embodiments -D is selected from the group consisting of cytotoxic/chemotherapeutic agents, immune checkpoint inhibitors or antagonists, immune checkpoint agonists, multi-specific drugs, antibody-drug conjugates (ADC), radionuclides or targeted radionuclide therapeutics, DNA damage repair inhibitors, tumor metabolism inhibitors, pattern recognition receptor agonists, protein kinase inhibitors, chemokine and chemoattractant receptor agonists, chemokine or chemokine receptor antagonists, cytokine receptor agonists, death receptor agonists, CD47 or SIRPa antagonists, oncolytic drugs, signal converter proteins, epigenetic modifiers, tumor peptides or tumor vaccines, heat shock protein (HSP) inhibitors, proteolytic enzymes, ubiquitin and proteasome inhibitors, adhesion molecule antagonists, and hormones including hormone peptides and synthetic hormones.

In certain embodiments -D is a growth hormone moiety, such as human growth hormone moiety.

In certain embodiments -D is a natriuretic peptide moiety, such as an ANP, BNP, CNP or DNP moiety. In certain embodiments -D is a CNP moiety.

In certain embodiments -D is a parathyroid hormone (PTH).

In certain embodiments -D is resiquimod.

In certain embodiments -D is doxorubicin.

In certain embodiments -D is an antibiotic drug moiety, such as an antibiotic drug moiety selected from the group consisting of aminoglycosides, tetracycline antibiotics, amphenicols, pleuromutilins, macrolid antibiotics, lincosamides, steroid antibiotics, antifolate antibiotics, sulfonamides, topoisomerase inhibitors, quinolones, fluoroquinolones, nitroimidazole antibiotics, nitrofuran antibiotics, rifamycins, glycopeptides, penicillins, cephalosporins, monobactams, beta-lactamase inhibitors, polymyxin antibiotics, lipopeptide antibiotics, oxazolidinon, antimicrobial peptides, antimicrobial proteins, porphyrins, azole antifungals, polyenes, antiprotozoal drugs, fosfomycin, cycloserine, and bacitracin.

In certain embodiments -D is selected from the group consisting of antisense RNA drug moieties, antisense DNA drug moieties, ribozyme drug moieties or RNAi drug moieties targeting a VEGF nucleic acid; anti-VEGF aptamer drug moieties, anti-VEGF antibody drug moieties, anti-VEGF antibody fragment drug moieties, DARPin drug moieties, and soluble VEGF receptor decoy drug moieties that prevent binding of a VEGF to its cognate receptor; antisense, ribozyme, and RNAi drug molecules targeting a cognate VEGF receptor (VEGFR) nucleic acid; anti-VEGFR aptamer drug moieties or anti-VEGFR antibodies that bind to a cognate VEGFR receptor; anti-VEGFR antibody fragment drug moieties that bind to a cognate VEGFR receptor and VEGFR tyrosine kinase inhibitors.

The moiety is conjugated to -D via a functional group of -D, which functional group is in certain embodiments selected from the group consisting of carboxylic acid, primary amine, secondary amine, thiol, sulfonic acid, carbonate, carbamate, hydroxyl, aldehyde, ketone, hydrazine, isothiocyanate, phosphoric acid, phosphonic acid, acryloyl, hydroxylamine, sulfate, vinyl sulfone, vinyl ketone, diazoalkane, guanidine, aziridine, amide, imide, imine, urea, amidine, guanidine, sulfonamide, phosphonamide, phorphoramide, hydrazide and selenol. In certain embodiments -L¹- is conjugated to -D via a functional group of -D selected from the group consisting of carboxylic acid, primary amine, secondary amine, thiol, sulfonic acid, carbonate, carbamate, hydroxyl, aldehyde, ketone, hydrazine, isothiocyanate, phosphoric acid, phosphonic acid, acryloyl, hydroxylamine, sulfate, vinyl sulfone, vinyl ketone, diazoalkane, guanidine, amidine and aziridine. In certain embodiments -L¹- is conjugated to -D via a functional group of -D selected from the group consisting of hydroxyl, primary amine, secondary amine, amidine and carboxylic acid. In certain embodiments -L¹- is conjugated to -D via a hydroxyl group of -D. In certain embodiments -L¹- is conjugated to -D via a primary amine group of -D. In certain embodiments -L¹- is conjugated to -D via a secondary amine group of -D. In certain embodiments -L¹- is conjugated to -D via a carboxylic acid group of -D. In certain embodiments -L¹- is conjugated to -D via an amidine group of -D.

The moiety -L¹- may be connected to -D through any type of linkage, provided that it is reversible. In certain embodiments -L¹- is connected to -D through a linkage selected from the group consisting of amide, ester, carbamate, acetal, aminal, imine, oxime, hydrazone, disulfide, acylguanidine, acylamidine, carbonate, phosphate, sulfate, urea, hydrazide, thioester, thiophosphate, thiosulfate, sulfonamide, sulfoamidine, sulfaguanidine, phosphoramide, phosphoamidine, phosphoguanidine, phosphonamide, phosphonamidine, phosphonguanidine, phosphonate, borate and imide. In certain embodiments -L¹- is connected to -D through a linkage selected from the group consisting of amide, ester, carbonate, carbamate, acetal, aminal, imine, oxime, hydrazone, disulfide, acylamidine and acylguanidine. In certain embodiments -L¹- is connected to -D through a linkage selected from the group consisting of amide, ester, carbonate, acylamide and carbamate. It is understood that some of these linkages may not be reversible per se, but that in the present invention neighboring groups present in -L¹- render these linkages reversible. In certain embodiments -L¹- is connected to -D through an ester linkage. In certain embodiments -L¹- is connected to -D through a carbonate linkage. In certain embodiments -L¹- is connected to -D through an acylamidine linkage. In certain embodiments -L¹- is connected to -D through a carbamate linkage. In certain embodiments -L¹- is connected to -D through an amide linkage.

Strictly speaking the term “-D” refers to a drug moiety as present in the conjugates of the present invention and it is understood that for simplicity the drug molecules of the complexes are not explicitly mentioned as “D-H” and “D-OH”, but are also covered by the term “-D”.

The moiety -L¹- is a linker moiety from which -D is released in its free form, i.e. in the form of D-H or D-OH. Such moieties are also known as “prodrug linkers” or “reversible prodrug linkers” and are known in the art, such as for example the reversible linker moieties disclosed in WO 2005/099768 A2, WO 2006/136586 A2, WO 2011/089216 A1, WO 2013/024053 A1, WO 2011/012722 A1, WO 2011/089214 A1, WO 2011/089215 A1, WO 2013/024052 A1 and WO 2013/160340 A1, which are incorporated by reference herewith.

In one embodiment -L¹- has a structure as disclosed in WO 2009/095479 A2. Accordingly, in one embodiment the moiety -L¹- is of formula (II):

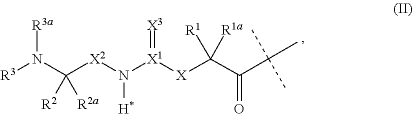

wherein the dashed line indicates attachment to a nitrogen of -D by forming an amide bond;
—X— is —C(R⁴R^{4a})—; —N(R⁴)—; —O—; —C(R⁴R^{4a})—C(R⁵R^{5a})—; —C(R⁵R^{5a})—C(R⁴R^{4a})—; —C(R⁴R^{4a})—N(R⁶)—; —N(R⁶)—C(R⁴R^{4a})—; —C(R⁴R^{4a})—O—; —O—C(R⁴R^{4a})—; or —C(R⁷R^{7a})—;
X¹ is C; or S(O);
—X²— is —C(R⁸R^{8a})—; or —C(R⁸R^{8a})—C(R⁹R^{9a})—;
═X³ is ═O; ═S; or ═N—CN;
—R¹, —R^{1a}, —R², —R^{2a}, —R⁴, —R^{4a}, —R⁵, —R^{5a}, —R⁶, —R⁸, —R^{8a}, —R⁹, —R^{9a} are independently selected from the group consisting of —H; and C₁₋₆ alkyl;
—R³, —R^{3a} are independently selected from the group consisting of —H; and C₁₋₆ alkyl, provided that in case one of —R³, —R^{3a} or both are other than —H they are connected to N to which they are attached through an SP³-hybridized carbon atom;
—R⁷ is —N(R¹⁰R^{10a}); or —NR¹⁰—(C═O)—R¹¹;
—R^{7a}, —R¹⁰, —R^{10a}, —R¹¹ are independently of each other —H; or C₁₋₆ alkyl;
optionally, one or more of the pairs —R^{1a}/—R^{4a}, —R^{1a}/—R^{5a}, —R^{1a}/—R^{7a}, —R^{4a}/—R^{5a}, —R^{8a}/—R^{9a} form a chemical bond;
optionally, one or more of the pairs —R¹/—R^{1a}, —R²/—R^{2a}, —R⁴/—R^{4a}, —R⁵/—R^{5a}, —R⁸/—R^{8a}, —R⁹/—R^{9a} are joined together with the atom to which they are attached to form a C₃₋₁₀ cycloalkyl; or 3- to 10-membered heterocyclyl;
optionally, one or more of the pairs —R¹/—R⁴, —R¹/—R⁵, —R¹/—R⁶, —R¹/—R^{7a}, —R⁴/—R⁵, —R⁴/—R⁶, —R⁸/—R⁹, —R²/—R³ are joined together with the atoms to which they are attached to form a ring A;
optionally, R³/R^{3a} are joined together with the nitrogen atom to which they are attached to form a 3- to 10-membered heterocycle;
A is selected from the group consisting of phenyl; naphthyl; indenyl; indanyl; tetralinyl; C₃₋₁₀ cycloalkyl; 3- to 10-membered heterocyclyl; and 8- to 11-membered heterobicyclyl; and
wherein is substituted with at least one -L²- and wherein -L¹- is optionally further substituted, provided that the hydrogen marked with the asterisk in formula (II) is not replaced by -L²- or a substituent.

Preferably -L¹- of formula (II) is substituted with one moiety -L²-.

In one embodiment -L¹- of formula (II) is not further substituted.

It is understood that if —R³/—R^{3a} of formula (II) are joined together with the nitrogen atom to which they are attached to form a 3- to 10-membered heterocycle, only such 3- to 10-membered heterocycles may be formed in which the atoms directly attached to the nitrogen are SP³-hybridized carbon atoms. In other words, such 3- to 10-membered heterocycle formed by —R³/—R^{3a} together with the nitrogen atom to which they are attached has the following structure:

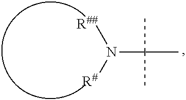

wherein
the dashed line indicates attachment to the rest of -L¹-;
the ring comprises 3 to 10 atoms comprising at least one nitrogen; and
R^{#} and R^{##} represent an SP³-hydridized carbon atom.

It is also understood that the 3- to 10-membered heterocycle may be further substituted.

Exemplary embodiments of suitable 3- to 10-membered heterocycles formed by —R³/—R^{3a} of formula (II) together with the nitrogen atom to which they are attached are the following:

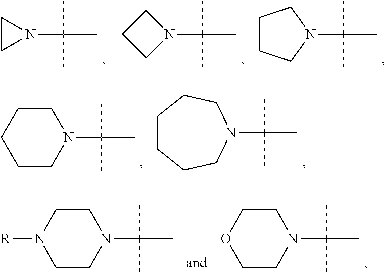

wherein
dashed lines indicate attachment to the rest of the molecule; and
—R is selected from the group consisting of —H and C₁₋₆ alkyl.

-L¹- of formula (II) may optionally be further substituted. In general, any substituent may be used as far as the cleavage principle is not affected, i.e. the hydrogen marked with the asterisk in formula (II) is not replaced and the nitrogen of the moiety

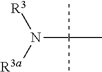

of formula (II) remains part of a primary, secondary or tertiary amine, i.e. —R³ and —R^{3a} are independently of each other —H or are connected to —N<through an SP³-hybridized carbon atom.

The nitrogen of -D linked to -L¹- of formula (II) is in certain embodiments the nitrogen of an amine functional group, which may be a primary, secondary or tertiary amine group. In certain embodiments the nitrogen of -D linked to -L¹- of formula (II) is the nitrogen of an amine functional group, which is a primary or secondary amine group. In certain embodiments the nitrogen of -D linked to -L¹- of formula (II) is the nitrogen of a primary amine functional group. In certain embodiments the nitrogen of -D linked to -L¹- of formula (II) is the nitrogen of a primary amine functional group. If -L¹- of formula (II) is conjugated to -D, wherein -D is a protein or peptide drug moiety the amine functional may in certain embodiments be the N-termina amine functional group or the amine functional group of a lysine site chain. If -L¹- of formula (II) is conjugated to -D, wherein -D is a protein or peptide drug moiety, the amine functional may in certain embodiments be the amine functional group of a lysine site chain.

In one embodiment —R¹ or —R^{1a} of formula (II) is substituted with -L²-. In another embodiment —R² or —R^{2a} of formula (II) is substituted with -L²-. In another embodiment —R³ or —R^{3a} of formula (II) is substituted with -L²-. In another embodiment —R⁴ of formula (II) is substituted with -L²-. In another embodiment —R⁵ or —R^{5a} of formula (II) is substituted with -L²-. In another embodiment —R⁶ of formula (II) is substituted with -L²-. In another embodiment —R⁷ or —R^{7a} of formula (II) is substituted with -L²-. In another embodiment —R⁸ or —R^{a} of formula (II) is substituted with -L²-. In another embodiment —R⁹ or —R^{9a} of formula (II) is substituted with -L²-.

In another embodiment -L¹- has a structure as disclosed in WO2016/020373A1. Accordingly, in another embodiment the moiety -L¹- is of formula (III):

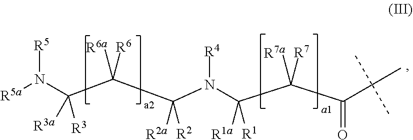

wherein
the dashed line indicates attachment to a primary or secondary amine or hydroxyl of -D by forming an amide or ester linkage, respectively;
—R¹, —R^{1a}, —R², —R^{2a}, —R³ and —R^{3a} are independently of each other selected from the group consisting of —H, —C(R⁸R^{8a}R^{8b}), —C(═O)R⁸, —C≡N, —C(═NR⁸)R^{8a}, —CR⁸(═CR^{8a}R^{8b}), —C≡CR⁸ and -T;
—R⁴, —R⁵ and —R^{5a} are independently of each other selected from the group consisting of —H, —C(R⁹R^{9a}R^{9b}) and -T;
a1 and a2 are independently of each other 0 or 1;
each —R⁶, —R^{6a}, —R⁷, —R^{7a}, —R^{8a}, —R^{8b}, —R⁹, —R^{9a}, R^{9b} are independently of each other selected from the group consisting of —H, halogen, —CN, —COOR¹⁰, —OR₁₀, —C(O)R¹⁰, —C(O)N(R¹⁰)R^{10a}), —S(O)₂N(R¹⁰R^{10a}), —S(O)N(R¹⁰R^{10a}), —S(O)₂R¹⁰, —S(O)R¹⁰, —N(R¹⁰)S(O)₂N(R^{10a}R^{10b}), —SR¹⁰, —N(R¹⁰R^{10a}), —NO₂, —OC(O)R¹⁰, —N(R¹⁰)C(O)R^{10a}, —N(R¹⁰)S(O)₂R^{10a}, —N(R¹⁰)S(O)R^{10a}, —N(R¹⁰)C(O)OR^{10a}, —N(R¹⁰)C(O)N(R^{10a}R^{10b}), —OC(O)N(R¹⁰R^{10a}), -T, C₁₋₂₀ alkyl, C₂₋₂₀ alkenyl, and C₂₋₂₀ alkynyl; wherein -T, C₁₋₂₀ alkyl, C₂₋₂₀ alkenyl, and C₂₋₂₀ alkynyl are optionally substituted with one or more —R¹¹, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)(R^{12a})—, —N(R¹²)C(O)N(R^{12a})—, and —OC(O)N(R¹²)—;
each —R¹⁰, —R^{10a}, R^{10b} is independently selected from the group consisting of —H, -T, C₁₋₂₀ alkyl, C₂₋₂₀ alkenyl, and C₂₋₂₀ alkynyl; wherein -T, C₁₋₂₀ alkyl, C₂₋₂₀ alkenyl, and C₂₋₂₀ alkynyl are optionally substituted with one or more —R¹¹, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)(R^{12a})—, —N(R¹²)C(O)N(R^{12a})—, and —OC(O)N(R¹²)—;
each T is independently of each other selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T is independently optionally substituted with one or more —R¹¹, which are the same or different;
each —R¹¹ is independently of each other selected from halogen, —CN, oxo (═O), —COOR¹³, —OR¹³, —C(O)R¹³), —C(O)N(R¹³R^{13a}), —S(O)₂N(R¹³R^{13a}), —S(O)N(R¹³R^{13a}), —S(O)₂R¹³, —S(O)R¹³, —N(R¹³)S(O)₂N(R^{13a}R^{13b}), —SR¹³, —N(R¹³R^{13a}), —NO₂, —OC(O)R¹³, —N(R¹³)C(O)R^{13a}, —N(R¹³)S(O)₂R^{13a}, —N(R¹³)S(O)R^{13a}, —N(R¹³)C(O)OR^{13a}, —N(R¹³)C(O)N(R^{13a}R^{13b}), —OC(O)N(R¹³R^{13a}), and C₁₋₆ alkyl; wherein C₁₋₆ alkyl is optionally substituted with one or more halogen, which are the same or different;
each —R¹², —R^{12a}, —R¹³, —R^{13a}, —R^{13b} is independently selected from the group consisting of —H, and C₁₋₆ alkyl; wherein C₁₋₆ alkyl is optionally substituted with one or more halogen, which are the same or different;
optionally, one or more of the pairs —R¹/—R^{1a}, —R²/—R^{2a}, —R³/—R^{3a}, —R⁶/—R^{6a}, —R⁷/—R^{7a} are joined together with the atom to which they are attached to form a C₃₋₁₀ cycloalkyl or a 3- to 10-membered heterocyclyl;
optionally, one or more of the pairs —R¹/—R², —R¹/—R³, —R¹/—R⁴, —R¹/—R⁵, —R¹/—R⁶, —R¹/—R⁷, —R²/—R³, —R²/—R⁴, —R²/—R⁵, —R²/—R⁶, —R²/—R⁷, —R³/—R⁴, —R³/—R⁵, —R³/—R⁶, —R³/—R⁷, —R⁴/—R⁵, —R⁴/—R⁶, —R⁴/—R⁷, —R⁵/—R⁶, —R⁵/—R⁷, —R⁶/—R⁷ are joint together with the atoms to which they are attached to form a ring A;
A is selected from the group consisting of phenyl; naphthyl; indenyl; indanyl; tetralinyl; C₃₋₁₀ cycloalkyl; 3- to 10-membered heterocyclyl; and 8- to 11-membered heterobicyclyl;
wherein is substituted with at least one -L²- and wherein -L¹- is optionally further substituted.

The optional further substituents of -L¹- of formula (III) are preferably as described above.

Preferably of formula (III) is substituted with one moiety -L²-.

In one embodiment -L¹- of formula (III) is not further substituted.

In another embodiment -L¹- has a structure as disclosed in EP1536334B1, WO2009/009712A1, WO2008/034122A1, WO2009/143412A2, WO2011/082368A2, and U.S. Pat. No. 8,618,124B2, which are herewith incorporated by reference.

In another embodiment -L¹- has a structure as disclosed in U.S. Pat. No. 8,946,405B2 and U.S. Pat. No. 8,754,190B2, which are herewith incorporated by reference. Accordingly, in another embodiment -L¹- is of formula (IV):

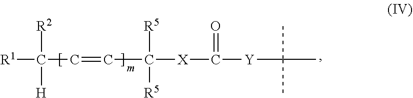

wherein
the dashed line indicates attachment to -D through a functional group of -D selected from the group consisting of —OH, —SH and —NH₂;
m is 0 or 1;
at least one or both of —R¹ and —R² is/are independently of each other selected from the group consisting of —CN, —NO₂, optionally substituted aryl, optionally substituted heteroaryl, optionally substituted alkenyl, optionally substituted alkynyl, —C(O)R³, —S(O)R³, —S(O)₂R³, and —SR⁴,
one and only one of —R¹ and —R² is selected from the group consisting of —H, optionally substituted alkyl, optionally substituted arylalkyl, and optionally substituted heteroarylalkyl;
—R³ is selected from the group consisting of —H, optionally substituted alkyl, optionally substituted aryl, optionally substituted arylalkyl, optionally substituted heteroaryl, optionally substituted heteroarylalkyl, —OR⁹ and —N(R⁹)₂;
—R⁴ is selected from the group consisting of optionally substituted alkyl, optionally substituted aryl, optionally substituted arylalkyl, optionally substituted heteroaryl, and optionally substituted heteroarylalkyl;
each —R⁵ is independently selected from the group consisting of —H, optionally substituted alkyl, optionally substituted alkenylalkyl, optionally substituted alkynylalkyl, optionally substituted aryl, optionally substituted arylalkyl, optionally substituted heteroaryl and optionally substituted heteroarylalkyl;
—R⁹ is selected from the group consisting of —H and optionally substituted alkyl;
—Y— is absent and —X— is —O— or —S—; or
—Y— is —N(Q)CH₂— and —X— is —O—;
Q is selected from the group consisting of optionally substituted alkyl, optionally substituted aryl, optionally substituted arylalkyl, optionally substituted heteroaryl and optionally substituted heteroarylalkyl;
optionally, —R¹ and —R² may be joined to form a 3 to 8-membered ring; and
optionally, both —R⁹ together with the nitrogen to which they are attached form a heterocyclic ring;
wherein -L¹- is substituted with at least one -L²- and wherein -L¹- is optionally further substituted.

Only in the context of formula (IV) the terms used have the following meaning:

The term “alkyl” as used herein includes linear, branched or cyclic saturated hydrocarbon groups of 1 to 8 carbons, or in some embodiments 1 to 6 or 1 to 4 carbon atoms.

The term “alkoxy” includes alkyl groups bonded to oxygen, including methoxy, ethoxy, isopropoxy, cyclopropoxy, cyclobutoxy, and similar.

The term “alkenyl” includes non-aromatic unsaturated hydrocarbons with carbon-carbon double bonds.

The term “alkynyl” includes non-aromatic unsaturated hydrocarbons with carbon-carbon triple bonds.

The term “aryl” includes aromatic hydrocarbon groups of 6 to 18 carbons, preferably 6 to 10 carbons, including groups such as phenyl, naphthyl, and anthracenyl. The term “heteroaryl” includes aromatic rings comprising 3 to 15 carbons containing at least one N, O or S atom, preferably 3 to 7 carbons containing at least one N, O or S atom, including groups such as pyrrolyl, pyridyl, pyrimidinyl, imidazolyl, oxazolyl, isoxazolyl, thiazolyl, isothiazolyl, quinolyl, indolyl, indenyl, and similar.

In some instance, alkenyl, alkynyl, aryl or heteroaryl moieties may be coupled to the remainder of the molecule through an alkylene linkage. Under those circumstances, the substituent will be referred to as alkenylalkyl, alkynylalkyl, arylalkyl or heteroarylalkyl, indicating that an alkylene moiety is between the alkenyl, alkynyl, aryl or heteroaryl moiety and the molecule to which the alkenyl, alkynyl, aryl or heteroaryl is coupled.

The term “halogen” includes bromo, fluoro, chloro and iodo.

The term “heterocyclic ring” refers to a 4 to 8 membered aromatic or non-aromatic ring comprising 3 to 7 carbon atoms and at least one N, O, or S atom. Examples are piperidinyl, piperazinyl, tetrahydropyranyl, pyrrolidine, and tetrahydrofuranyl, as well as the exemplary groups provided for the term “heteroaryl” above.

When a ring system is optionally substituted, suitable substituents are selected from the group consisting of alkyl, alkenyl, alkynyl, or an additional ring, each optionally further substituted. Optional substituents on any group, including the above, include halo, nitro, cyano, —OR, —SR, —NR₂, —OCOR, —NRCOR, —COOR, —CONR₂, —SOR, —SO₂R, —SONR₂, —SO₂NR₂, wherein each R is independently alkyl, alkenyl, alkynyl, aryl or heteroaryl, or two R groups taken together with the atoms to which they are attached form a ring.

Preferably -L¹- of formula (IV) is substituted with one moiety -L²-.

In another embodiment -L¹- has a structure as disclosed in WO2013/036857A1, which is herewith incorporated by reference. Accordingly, in another embodiment -L¹- is of formula (V):

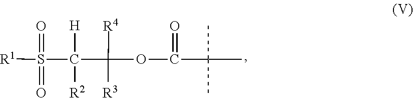

wherein
the dashed line indicates attachment to -D through an amine functional group of -D;
—R¹ is selected from the group consisting of optionally substituted C₁-C₆ linear, branched, or cyclic alkyl; optionally substituted aryl; optionally substituted heteroaryl; alkoxy; and —NR⁵₂;
—R² is selected from the group consisting of —H; optionally substituted C₁-C₆ alkyl; optionally substituted aryl; and optionally substituted heteroaryl;
—R³ is selected from the group consisting of —H; optionally substituted C₁-C₆ alkyl; optionally substituted aryl; and optionally substituted heteroaryl;
—R⁴ is selected from the group consisting of —H; optionally substituted C₁-C₆ alkyl; optionally substituted aryl; and optionally substituted heteroaryl;
each —R⁵ is independently of each other selected from the group consisting of —H; optionally substituted C₁-C₆ alkyl; optionally substituted aryl; and optionally substituted heteroaryl; or when taken together two —R⁵ can be cycloalkyl or cycloheteroalkyl;
wherein -L¹- is substituted with at least one -L²- and wherein -L¹- is optionally further substituted.

Only in the context of formula (V) the terms used have the following meaning:

“Alkyl”, “alkenyl”, and “alkynyl” include linear, branched or cyclic hydrocarbon groups of 1-8 carbons or 1-6 carbons or 1-4 carbons wherein alkyl is a saturated hydrocarbon, alkenyl includes one or more carbon-carbon double bonds and alkynyl includes one or more carbon-carbon triple bonds. Unless otherwise specified these contain 1-6 carbon atoms.

“Aryl” includes aromatic hydrocarbon groups of 6-18 carbons, preferably 6-10 carbons, including groups such as phenyl, naphthyl, and anthracene. “Heteroaryl” includes aromatic rings comprising 3-15 carbons containing at least one N, O or S atom, preferably 3-7 carbons containing at least one N, O or S atom, including groups such as pyrrolyl, pyridyl, pyrimidinyl, imidazolyl, oxazolyl, isoxazolyl, thiszolyl, isothiazolyl, quinolyl, indolyl, indenyl, and similar. The term “substituted” means an alkyl, alkenyl, alkynyl, aryl, or heteroaryl group comprising one or more substituent groups in place of one or more hydrogen atoms. Substituents may generally be selected from halogen including F, Cl, Br, and I; lower alkyl including linear, branched, and cyclic; lower haloalkyl including fluoroalkyl, chloroalkyl, bromoalkyl, and iodoalkyl; OH; lower alkoxy including linear, branched, and cyclic; SH; lower alkylthio including linear, branched and cyclic; amino, alkylamino, dialkylamino, silyl including alkylsilyl, alkoxysilyl, and arylsilyl; nitro; cyano; carbonyl; carboxylic acid, carboxylic ester, carboxylic amide, aminocarbonyl; aminoacyl; carbamate; urea; thiocarbamate; thiourea; ketone; sulfone; sulfonamide; aryl including phenyl, naphthyl, and anthracenyl; heteroaryl including 5-member heteroaryls including as pyrrole, imidazole, furan, thiophene, oxazole, thiazole, isoxazole, isothiazole, thiadiazole, triazole, oxadiazole, and tetrazole, 6-member heteroaryls including pyridine, pyrimidine, pyrazine, and fused heteroaryls including benzofuran, benzothiophene, benzoxazole, benzimidazole, indole, benzothiazole, benzisoxazole, and benzisothiazole.

Preferably -L¹- of formula (V) is substituted with one moiety -L²-.

In another embodiment -L¹- has a structure as disclosed in U.S. Pat. No. 7,585,837B2, which is herewith incorporated by reference. Accordingly, in another embodiment -L¹- is of formula (VI):

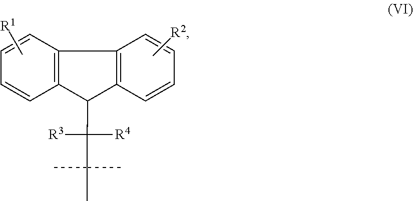

wherein
the dashed line indicates attachment to -D through an amine functional group of -D;
R¹ and R² are independently selected from the group consisting of hydrogen, alkyl, alkoxy, alkoxyalkyl, aryl, alkaryl, aralkyl, halogen, nitro, —SO₃H, —SO₂NHR⁵, amino, ammonium, carboxyl, PO₃H₂, and OPO₃H₂;
R³, R⁴, and R⁵ are independently selected from the group consisting of hydrogen, alkyl, and aryl;
wherein -L¹- is substituted with at least one -L²- and wherein -L¹- is optionally further substituted.

Suitable substituents for formulas (VI) are alkyl (such as C₁₋₆ alkyl), alkenyl (such as C₂₋₆ alkenyl), alkynyl (such as C₂₋₆ alkynyl), aryl (such as phenyl), heteroalkyl, heteroalkenyl, heteroalkynyl, heteroaryl (such as aromatic 4 to 7 membered heterocycle) or halogen moieties.

Only in the context of formula (VI) the terms used have the following meaning:

The terms “alkyl”, “alkoxy”, “alkoxyalkyl”, “aryl”, “alkaryl” and “aralkyl” mean alkyl radicals of 1-8, preferably 1-4 carbon atoms, e.g. methyl, ethyl, propyl, isopropyl and butyl, and aryl radicals of 6-10 carbon atoms, e.g. phenyl and naphthyl. The term “halogen” includes bromo, fluoro, chloro and iodo.

Preferably -L¹- of formula (VI) is substituted with one moiety -L²-.

In another embodiment -L¹- has a structure as disclosed in WO2002/089789A1, which is herewith incorporated by reference. Accordingly, in another embodiment -L¹- is of formula (VII):

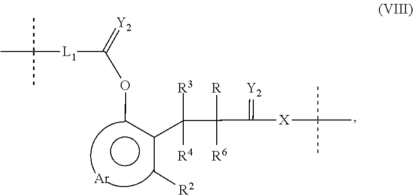

wherein
the dashed line indicates attachment to -D through an amine functional group of -D;
L₁ is a bifunctional linking group,
Y₁ and Y₂ are independently O, S or NR⁷;
R², R³, R⁴, R⁵, R⁶ and R⁷ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy, and C₁₋₆ heteroalkoxy;
Ar is a moiety which when included in formula (VII) forms a multisubstituted aromatic hydrocarbon or a multi-substituted heterocyclic group;
X is a chemical bond or a moiety that is actively transported into a target cell, a hydrophobic moiety, or a combination thereof,
y is 0 or 1;
wherein -L¹- is substituted with at least one -L²- and wherein -L¹- is optionally further substituted.

Only in the context of formula (VII) the terms used have the following meaning:

The term “alkyl” shall be understood to include, e.g. straight, branched, substituted C₁₋₁₂ alkyls, including alkoxy, C₃₋₈ cycloalkyls or substituted cycloalkyls, etc.

The term “substituted” shall be understood to include adding or replacing one or more atoms contained within a functional group or compounds with one or more different atoms.

Substituted alkyls include carboxyalkyls, aminoalkyls, dialkylaminos, hydroxyalkyls and mercaptoalkyls; substitued cycloalkyls include moieties such as 4-chlorocyclohexyl; aryls include moieties such as napthyl; substituted aryls include moieties such as 3-bromo-phenyl; aralkyls include moieties such as toluyl; heteroalkyls include moieties such as ethylthiophene; substituted heteroalkyls include moieties such as 3-methoxythiophone; alkoxy includes moieities such as methoxy; and phenoxy includes moieties such as 3-nitrophenoxy. Halo- shall be understood to include fluoro, chloro, iodo and bromo.

Preferably -L¹- of formula (VII) is substituted with one moiety -L²-.

In another embodiment -L¹- comprises a substructure of formula (VIII)

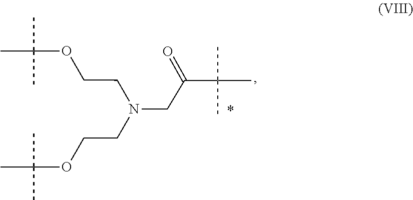

wherein
the dashed line marked with the asterisk indicates attachment to a nitrogen of -D by forming an amide bond;
the unmarked dashed lines indicate attachment to the remainder of -L¹-; and
wherein -L¹- is substituted with at least one -L²- and wherein -L¹- is optionally further substituted.

Preferably -L¹- of formula (VIII) is substituted with one moiety -L²-.

In one embodiment -L¹- of formula (VIII) is not further substituted.

In another embodiment -L¹- comprises a substructure of formula (IX)

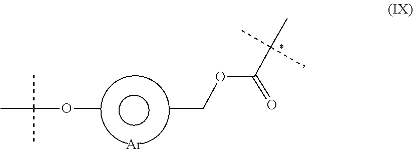

wherein
the dashed line marked with the asterisk indicates attachment to a nitrogen of -D by forming a carbamate bond;
the unmarked dashed lines indicate attachment to the remainder of -L¹-; and
wherein -L¹- is substituted with at least one -L²- and wherein -L¹- is optionally further substituted.

Preferably -L¹- of formula (IX) is substituted with one moiety -L²-.

In one embodiment -L¹- of formula (IX) is not further substituted.

In one embodiment -L¹- is of formula (IX-a):

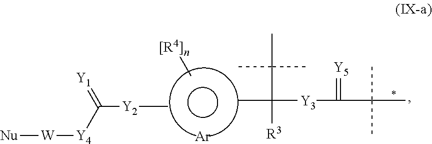

wherein
the dashed line marked with the asterisk indicates attachment to a nitrogen of -D by forming an amide bond and the unmarked dashed line indicates attachment to -L²-;
n is 0, 1, 2, 3, or 4;
═Y₁, ═Y₅ are independently of each other selected from the group consisting of ═O and ═S;
—Y₂—, —Y₃— are independently of each other selected from the group consisting of —O— and —S—;
—Y₄— is selected from the group consisting of —O—, —NR⁵— and —C(R⁶R^{6a})—;
—R³, —R⁵, —R⁶, —R^{6a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;
—R⁴ is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;
—W— is selected from the group consisting of C₁₋₂₀ alkyl optionally interrupted by one or more groups selected from the group consisting of C₃₋₁₀ cycloalkyl, 8- to 30-membered carbopolycyclyl, 3- to 10-membered heterocyclyl, —C(O)—, —C(O)N(R⁷)—, —O—, —S— and —N(R⁷)—;
—Nu is a nucleophile selected from the group consisting of —N(R⁷R^{7a}), —N(R⁷OH), —N(R⁷)—N(R^{7a}R^{7b}), —S(R⁷), —COOH,

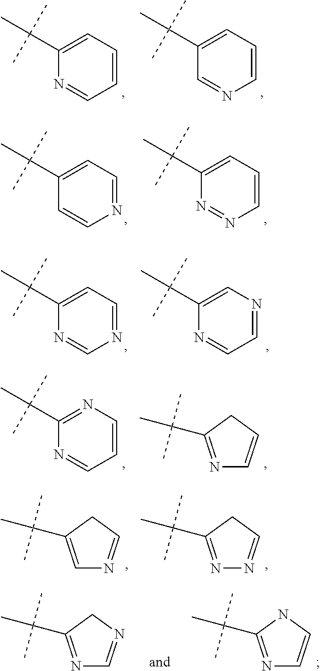

—Ar— is selected from the group consisting of

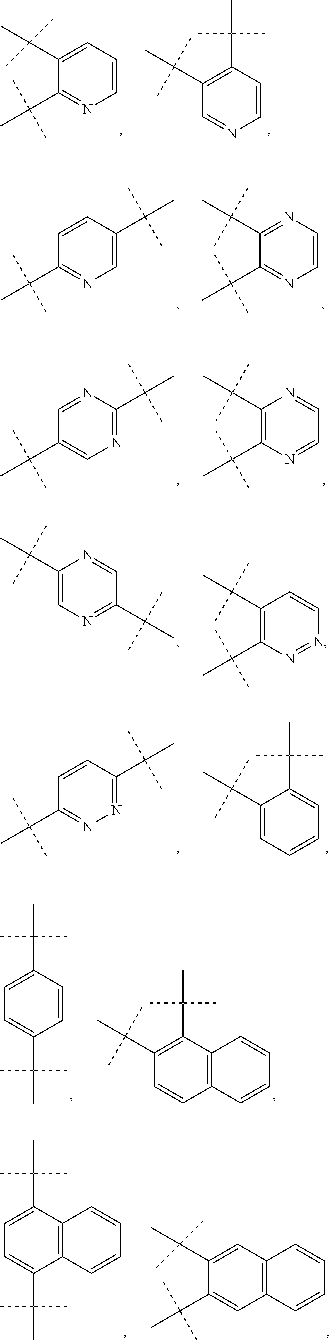

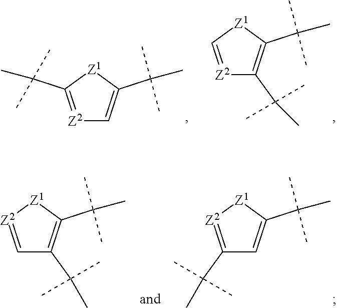

wherein
dashed lines indicate attachment to the remainder of -L¹-,
—Z¹— is selected from the group consisting of —O—, —S— and —N(R⁷)—, and
—Z²— is —N(R⁷)—; and
—R⁷, —R^{7a}, —R^{7b} are independently of each other selected from the group consisting of —H, C₁₋₆ alkyl, C₂₋₆ alkenyl and C₂₋₆ alkynyl;
wherein -L¹- is optionally further substituted.

In one embodiment -L¹- of formula (IX-a) is not further substituted.

In another embodiment -L¹- is of formula (IX-b):

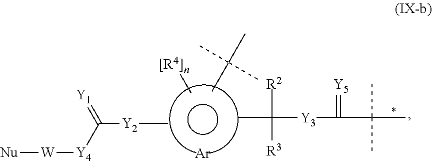

wherein
the dashed line marked with the asterisk indicates attachment to a nitrogen of -D by forming an amide bond and the unmarked dashed line indicates attachment to -L²-;
n is 0, 1, 2, 3, or 4;
═Y₁, ═Y₅ are independent of each other selected from the group consisting of ═O and ═S;
—Y₂—, —Y₃— are independently of each other selected from the group consisting of —O— and —S—;
—Y₄— is selected from the group consisting of —O—, —NR⁵— and —C(R⁶R^{6a})—;
—R², —R³, —R⁵, —R⁶, —R^{6a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;
—R⁴ is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;
—W— is selected from the group consisting of C₁₋₂₀ alkyl optionally interrupted by one or more groups selected from the group consisting of C₃₋₁₀ cycloalkyl, 8- to 30-membered carbopolycyclyl, 3- to 10-membered heterocyclyl, —C(O)—, —C(O)N(R⁷)—, —O—, —S— and —N(R⁷)—;
-Nu is a nucleophile selected from the group consisting of —N(R⁷R^{7a}), —N(R⁷OH), —N(R⁷)—N(R^{7a}R^{7b}), —S(R⁷), —COOH,

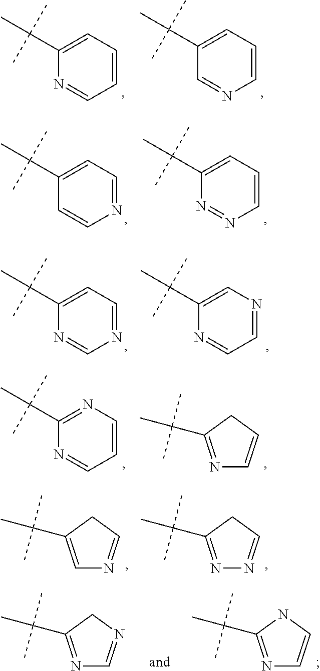

—Ar— is selected from the group consisting of

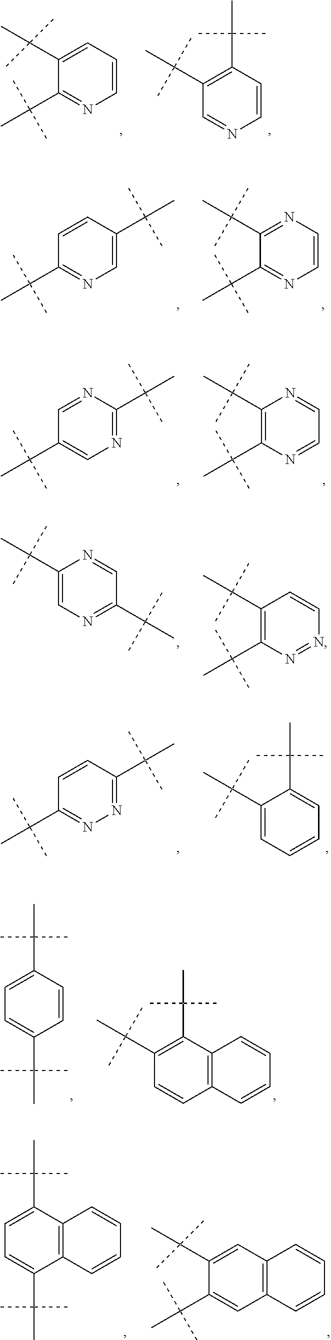

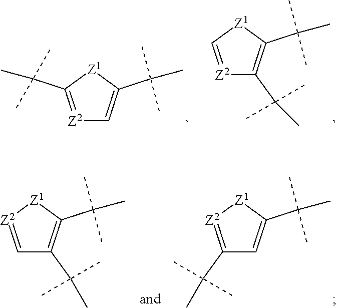

wherein
dashed lines indicate attachment to the remainder of -L¹-,
—Z¹— is selected from the group consisting of —O—, —S— and —N(R⁷)—, and
—Z²— is —N(R⁷)—; and
—R⁷, —R^{7a}, —R^{7b} are independently of each other selected from the group consisting of —H, C₁₋₆ alkyl, C₂₋₆ alkenyl and C₂₋₆ alkynyl;
wherein -L¹- is optionally further substituted.

In one embodiment -L¹- of formula (IX-b) is not further substituted.

In certain embodiments -L¹- has a structure as disclosed in WO2020/206358 A1. Accordingly, in certain embodiments the moiety -L¹- is of formula (X):

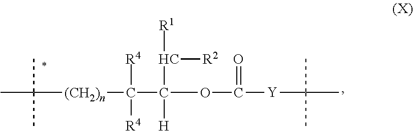

wherein
the unmarked dashed line indicates attachment to -D;
the dashed line marked with the asterisk indicates attachment to -L²-;
n is an integer selected from the group consisting of 0, 1, 2, 3, 4, 5 and 6;
—R¹ and —R² are independently an electron-withdrawing group, alkyl, or —H, and wherein at least one of —R¹ or —R² is an electron-withdrawing group;
each —R⁴ is independently C₁-C₃ alkyl or the two —R⁴ are taken together with the carbon atom to which they are attached to form a 3- to 6-membered ring; and
—Y— is absent when -D is a drug moiety connected through an amine, or —Y— is —N(R⁶)CH₂— when -D is a drug moiety connected through a phenol, alcohol, thiol, thiophenol, imidazole, or non-basic amine; wherein —R⁶ is optionally substituted C₁-C₆ alkyl, optionally substituted aryl, or optionally substituted heteroaryl.

In certain embodiments n of formula (X) is an integer selected from 1, 2, 3, 4, 5 and 6. In certain embodiments n of formula (X) is an integer selected from 1, 2 and 3. In certain embodiments n of formula (X) is an integer from 0, 1, 2 and 3. In certain embodiments n of formula (X) is 1. In certain embodiments n of formula (X) is 2. In certain embodiments n of formula (X) is 3.

In certain embodiments the electron-withdrawing group of —R¹ and —R² of formula (X) is selected from the group consisting of —CN; —NO₂; optionally substituted aryl; optionally substituted heteroaryl; optionally substituted alkenyl; optionally substituted alkynyl; —COR³, —SOR³, or —SO₂R³, wherein —R³ is —H, optionally substituted alkyl, optionally substituted aryl, optionally substituted arylalkyl, optionally substituted heteroaryl, optionally substituted heteroarylalkyl, —OR⁸ or —NR⁸₂, wherein each —R⁸ is independently —H or optionally substituted alkyl, or both —R⁸ groups are taken together with the nitrogen to which they are attached to form a heterocyclic ring; or —SR⁹, wherein —R⁹ is optionally substituted alkyl, optionally substituted aryl, optionally substituted arylalkyl, optionally substituted heteroaryl, or optionally substituted heteroarylalkyl.

In certain embodiments the electron-withdrawing group of —R¹ and —R² of formula (X) is —CN. In certain embodiments the electron-withdrawing group of —R¹ and —R² of formula (X) is —NO₂. In certain embodiments the electron-withdrawing group of —R¹ and —R² of formula (X) is optionally substituted aryl comprising 6 to 10 carbons. In certain embodiments the electron-withdrawing group of —R¹ and —R² of formula (X) is optionally substituted phenyl, naphthyl, or anthracenyl. In certain embodiments the electron-withdrawing group of —R¹ and —R² of formula (X) is optionally substituted heteroaryl comprising 3 to 7 carbons and comprising at least one N, O, or S atom. In certain embodiments the electron-withdrawing group of —R¹ and —R² of formula (X) is optionally substituted pyrrolyl, pyridyl, pyrimidinyl, imidazolyl, oxazolyl, isoxazolyl, thiazolyl, isothiazolyl, quinolyl, indolyl, or indenyl. In certain embodiments the electron-withdrawing group of —R¹ and —R² of formula (X) is optionally substituted alkenyl containing 2 to 20 carbon atoms. In certain embodiments the electron-withdrawing group of —R¹ and —R² of formula (X) is optionally substituted alkynyl comprising 2 to 20 carbon atoms. In certain embodiments the electron-withdrawing group of —R¹ and —R² of formula (X) is —COR³, —SOR³, or —SO₂R³, wherein —R³ is —H, optionally substituted alkyl comprising 1 to 20 carbon atoms, optionally substituted aryl, optionally substituted arylalkyl, optionally substituted heteroaryl, optionally substituted heteroarylalkyl, —OR⁸ or —NR⁸₂, wherein each —R⁸ is independently —H or optionally substituted alkyl comprising 1 to 20 carbon atoms, or both —R⁸ groups are taken together with the nitrogen to which they are attached to form a heterocyclic ring. In certain embodiments the electron-withdrawing group of —R¹ and —R² of formula (X) is —SR⁹, wherein —R⁹ is optionally substituted alkyl comprising 1 to 20 carbon atoms, optionally substituted aryl, optionally substituted arylalkyl, optionally substituted heteroaryl, or optionally substituted heteroarylalkyl.

In certain embodiments at least one of —R¹ or —R² of formula (X) is —CN, —SOR³ or —SO₂R³. In certain embodiments at least one of —R¹ and —R² of formula (X) is —CN or —SO₂R³. In certain embodiments at least one of —R¹ and —R² of formula (X) is —CN or —SO₂R³, wherein —R³ is optionally substituted alkyl, optionally substituted aryl, or —NR⁸₂. In certain embodiments at least one of —R¹ and —R² of formula (X) is —CN, —SO₂N(CH₃)₂, —SO₂CH₃, phenyl substituted with —SO₂, phenyl substituted with —SO₂ and —Cl, —SO₂N(CH₂CH₂)₂O, —SO₂CH(CH₃)₂, —SO₂N(CH₃)(CH₂CH₃), or —SO₂N(CH₂CH₂OCH₃)₂.

In certain embodiments each —R⁴ of formula (X) is independently C₁-C₃ alkyl. In certain embodiments both —R⁴ are methyl.

In certain embodiments —Y— of formula (X) is absent. In certain embodiments —Y— of formula (X) is —N(R⁶)CH₂—.

In certain embodiments -L¹- is of formula (X), wherein n is 1, —R¹ is —CN, —R² is —H, and —R⁴ is —CH₃. In certain embodiments -L¹- is of formula (X), wherein n is 1, —R¹ is —SO₂N(CH₃)₂, —R² is —H, and —R⁴ is —CH₃. In certain embodiments -L¹- is of formula (X), wherein n is 1, —R¹ is SO₂CH₃, —R² is —H, and —R⁴ is —CH₃. In certain embodiments -L¹- is of formula (X), wherein n is 1, —R¹ is —SO₂N(CH₂CH₂)₂CHCH₃, —R² is —H, and —R⁴ is —CH₃. In certain embodiments -L¹- is of formula (X), wherein n is 1, —R¹ is phenyl substituted with —SO₂, —R² is —H, and —R⁴ is —CH₃. In certain embodiments -L¹- is of formula (X), wherein n is 1, —R¹ is phenyl substituted with —SO₂ and —Cl, —R² is —H, and —R⁴ is —CH₃. In certain embodiments -L¹- is of formula (X), wherein n is 1, —R¹ is —SO₂N(CH₂CH₂)₂O, —R² is —H, and —R⁴ is —CH₃. In certain embodiments -L¹- is of formula (X), wherein n is 1, —R¹ is —SO₂CH(CH₃)₂, —R² is —H, and —R⁴ is —CH₃. In certain embodiments -L¹- is of formula (X), wherein n is 1, —R¹ is —SO₂N(CH₃)(CH₂CH₃), —R² is —H, and —R⁴ is —CH₃. In certain embodiments -L¹- is of formula (X), wherein n is 1, —R¹ is —SO₂N(CH₂CH₂OCH₃)₂, —R² is —H, and —R⁴ is —CH₃. In certain embodiments -L¹- is of formula (X), wherein n is 1, —R¹ is phenyl substituted with —SO₂ and —CH₃, —R² is —H, and —R⁴ is —CH₃.

In certain embodiments -L¹- is of formula (X), wherein n is 2, —R¹ is —CN, —R² is —H, and —R⁴ is —CH₃. In certain embodiments -L¹- is of formula (X), wherein n is 2, —R¹ is —SO₂N(CH₃)₂, —R² is —H, and —R⁴ is —CH₃. In certain embodiments -L¹- is of formula (X), wherein n is 2, —R¹ is SO₂CH₃, —R² is —H, and —R⁴ is —CH₃. In certain embodiments -L¹- is of formula (X), wherein n is 2, —R¹ is —SO₂N(CH₂CH₂)₂CHCH₃, —R² is —H, and —R⁴ is —CH₃. In certain embodiments -L¹- is of formula (X), wherein n is 2, —R¹ is phenyl substituted with —SO₂, —R² is —H, and —R⁴ is —CH₃. In certain embodiments -L¹- is of formula (X), wherein n is 2, —R¹ is phenyl substituted with —SO₂ and —Cl, —R² is —H, and —R⁴ is —CH₃. In certain embodiments -L¹- is of formula (X), wherein n is 2, —R¹ is —SO₂N(CH₂CH₂)₂O, —R² is —H, and —R⁴ is —CH₃. In certain embodiments -L¹- is of formula (X), wherein n is 2, —R¹ is —SO₂CH(CH₃)₂, —R² is —H, and —R⁴ is —CH₃. In certain embodiments -L¹- is of formula (X), wherein n is 2, —R¹ is —SO₂N(CH₃)(CH₂CH₃), —R² is —H, and —R⁴ is —CH₃. In certain embodiments -L¹- is of formula (X), wherein n is 2, —R¹ is —SO₂N(CH₂CH₂OCH₃)₂, —R² is —H, and —R⁴ is —CH₃. In certain embodiments -L¹- is of formula (X), wherein n is 2, —R¹ is phenyl substituted with —SO₂ and —CH₃, —R² is —H, and —R⁴ is —CH₃.

In certain embodiments -L¹- is of formula (X), wherein n is 3, —R¹ is —CN, —R² is —H, and —R⁴ is —CH₃. In certain embodiments -L¹- is of formula (X), wherein n is 3, —R¹ is —SO₂N(CH₃)₂, —R² is —H, and —R⁴ is —CH₃. In certain embodiments -L¹- is of formula (X), wherein n is 3, —R¹ is SO₂CH₃, —R² is —H, and —R⁴ is —CH₃. In certain embodiments -L¹- is of formula (X), wherein n is 3, —R¹ is —SO₂N(CH₂CH₂)₂CHCH₃, —R² is —H, and —R⁴ is —CH₃. In certain embodiments -L¹- is of formula (X), wherein n is 3, —R¹ is phenyl substituted with —SO₂, —R² is —H, and —R⁴ is —CH₃. In certain embodiments -L¹- is of formula (X), wherein n is 3, —R¹ is phenyl substituted with —SO₂ and —Cl, —R² is —H, and —R⁴ is —CH₃. In certain embodiments -L¹- is of formula (X), wherein n is 3, —R¹ is —SO₂N(CH₂CH₂)₂O, —R² is —H, and —R⁴ is —CH₃. In certain embodiments -L¹- is of formula (X), wherein n is 3, —R¹ is —SO₂CH(CH₃)₂, —R² is —H, and —R⁴ is —CH₃. In certain embodiments -L¹- is of formula (X), wherein n is 3, —R¹ is —SO₂N(CH₃)(CH₂CH₃), —R² is —H, and —R⁴ is —CH₃. In certain embodiments -L¹- is of formula (X), wherein n is 3, —R¹ is —SO₂N(CH₂CH₂OCH₃)₂, —R² is —H, and —R⁴ is —CH₃. In certain embodiments -L¹- is of formula (X), wherein n is 3, —R¹ is phenyl substituted with —SO₂ and —CH₃, —R² is —H, and —R⁴ is —CH₃.

Only in the context of formula (X) the terms used have the following meaning:

The term “alkyl” refers to linear, branched, or cyclic saturated hydrocarbon groups of 1 to 20, 1 to 12, 1 to 8, 1 to 6, or 1 to 4 carbon atoms. In certain embodiments an alkyl is linear or branched. Examples of linear or branched alkyl groups include methyl, ethyl, n-propyl, isopropyl, n-butyl, t-butyl, isobutyl, sec-butyl, n-pentyl, n-hexyl, n-heptyl, n-octyl, n-nonyl, and n-decyl. In certain embodiments an alkyl is cyclic. Examples of cyclic alkyl groups include cyclopropyl, cyclobutyl, cyclopentyl, cyclopentadienyl, and cyclohexyl.

The term “alkoxy” refers to alkyl groups bonded to oxygen, including methoxy, ethoxy, isopropoxy, cyclopropoxy, and cyclobutoxy.

The term “alkenyl” refers to non-aromatic unsaturated hydrocarbons with carbon-carbon double bonds and 2 to 20, 2 to 12, 2 to 8, 2 to 6, or 2 to 4 carbon atoms.

The term “alkynyl” refers to non-aromatic unsaturated hydrocarbons with carbon-carbon triple bonds and 2 to 20, 2 to 12, 2 to 8, 2 to 6, or 2 to 4 carbon atoms.

The term “aryl” refers to aromatic hydrocarbon groups of 6 to 18 carbons, preferably 6 to 10 carbons, including groups such as phenyl, naphthyl, and anthracenyl. The term “heteroaryl” refers to aromatic rings comprising 3 to 15 carbons comprising at least one N, O or S atom, preferably 3 to 7 carbons comprising at least one N, O or S atom, including groups such as pyrrolyl, pyridyl, pyrimidinyl, imidazolyl, oxazolyl, isoxazolyl, thiazolyl, isothiazolyl, quinolyl, indolyl, and indenyl.

In certain embodiments alkenyl, alkynyl, aryl or heteroaryl moieties may be coupled to the remainder of the molecule through an alkyl linkage. Under those circumstances, the substituent will be referred to as alkenylalkyl, alkynylalkyl, arylalkyl or heteroarylalkyl, indicating that an alkylene moiety is between the alkenyl, alkynyl, aryl or heteroaryl moiety and the molecule to which the alkenyl, alkynyl, aryl or heteroaryl is coupled.

The term “halogen” or “halo” refers to bromo, fluoro, chloro and iodo.

The term “heterocyclic ring” or “heterocyclyl” refers to a 3- to 15-membered aromatic or non-aromatic ring comprising at least one N, O, or S atom. Examples include piperidinyl, piperazinyl, tetrahydropyranyl, pyrrolidine, and tetrahydrofuranyl, as well as the exemplary groups provided for the term “heteroaryl” above. In certain embodiments a heterocyclic ring or heterocyclyl is non-aromatic. In certain embodiments a heterocyclic ring or heterocyclyl is aromatic.

The term “optionally substituted” refers to a group may be unsubstituted or substituted by one or more (e.g., 1, 2, 3, 4 or 5) of the substituents which may be the same or different. Examples of substituents include alkyl, alkenyl, alkynyl, halogen, —CN, —OR^{aa}, —SR^{aa}, —NR^{aa}R^{bb}, —NO₂, —C═NH(OR^{aa}), —C(O)R^{aa}, —OC(O)R^{aa}, —C(O)OR^{aa}, —C(O)NR^{aa}R^{bb}, —OC(O)NR^{aa}R^{bb}, —NR^{aa}C(O)R^{bb}, —NR^{aa}C(O)OR^{bb}, —S(O)R^{aa}, —S(O)₂R^{aa}, —NR^{aa}S(O)R^{bb}, —C(O)NR^{aa}S(O)R^{bb}, —NR^{aa}S(O)₂R^{bb}, —C(O)NR^{aa}S(O)₂R^{bb}, —S(O)NR^{aa}R^{bb}, —S(O)₂NR^{aa}R^{bb}, —P(O)(OR^{aa})(OR^{bb}), heterocyclyl, heteroaryl, or aryl, wherein the alkyl, alkenyl, alkynyl, cycloalkyl, heterocyclyl, heteroaryl, and aryl are each independently optionally substituted by —R^{cc}, wherein —R^{aa} and —R^{bb} are each independently —H, alkyl, alkenyl, alkynyl, heterocyclyl, heteroaryl, or aryl, or —R^{aa} and —R^{bb} are taken together with the nitrogen atom to which they attach to form a heterocyclyl, which is optionally substituted by alkyl, alkenyl, alkynyl, halogen, hydroxyl, alkoxy, or —CN, and wherein: each —R^{cc} is independently alkyl, alkenyl, alkynyl, halogen, heterocyclyl, heteroaryl, aryl, —CN, or —NO₂.

In one embodiment -L²- is a chemical bond.

In another embodiment -L²- is a spacer moiety.

When -L²- is other than a chemical bond, -L²- is preferably selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;

—R^{y1} and —R^{y1a} are independently of each other selected from the group consisting of —H, -T, C₁₋₅₀ alkyl, C₂₋₅₀ alkenyl, and C₂₋₅₀ alkynyl; wherein -T, C₁₋₅₀ alkyl, C₂₋₅₀ alkenyl, and C₂₋₅₀ alkynyl are optionally substituted with one or more —R^{y2}, which are the same or different, and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;

each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;

each —R^{y2} is independently selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), (O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and each —R^{y3}, —R^{y3a}, —R^{y4}, —R^{y4a}, —R^{y5}, —R^{y5a} and —R^{y5b} is independently selected from the group consisting of —H, and C₁₋₆ alkyl, wherein C₁₋₆ alkyl is optionally substituted with one or more halogen, which are the same or different.

When -L²- is other than a single chemical bond, -L²- is more preferably selected from -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₂₀ alkyl, C₂₋₂₀ alkenyl, and C₂₋₂₀ alkynyl are optionally substituted with one or more —R^{y2}, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;

—R^{y1} and —R^{y1a} are independently of each other selected from the group consisting of —H, -T, C₁₋₁₀ alkyl, C₂₋₁₀ alkenyl, and C₂₋₁₀ alkynyl; wherein -T, C₁₋₁₀ alkyl, C₂₋₁₀ alkenyl, and C₂₋₁₀ alkynyl are optionally substituted with one or more —R^{y2}, which are the same or different, and wherein C₁₋₁₀ alkyl, C₂₋₁₀ alkenyl, and C₂₋₁₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;

each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;

—R^{y2} is selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R⁵, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and

each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C₁₋₆ alkyl is optionally substituted with one or more halogen, which are the same or different.

When -L²- is other than a single chemical bond, -L²- is even more preferably selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;

—R^{y1} and —R^{y1a} are independently selected from the group consisting of —H, -T, C₁₋₁₀ alkyl, C₂₋₁₀ alkenyl, and C₂₋₁₀ alkynyl;

each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl;

each —R^{y2} is independently selected from the group consisting of halogen, and C₁₋₆ alkyl; and

each —R^{y3}, —R^{y3a}, —R^{y4}, —R^{y4a}, —R^{y5}, —R^{y5a} and —R^{y5b} is independently of each other selected from is independently of each other selected from the group consisting of —H, and C₁₋₆ alkyl; wherein C₁₋₆ alkyl is optionally substituted with one or more halogen, which are the same or different.

Even more preferably, -L²- is a C₁₋₂₀ alkyl chain, which is optionally interrupted by one or more groups independently selected from —O—, -T- and —C(O)N(R^{y1})—; and which C₁₋₂₀ alkyl chain is optionally substituted with one or more groups independently selected from —OH, -T and —C(O)N(R^{y6}R^{y6a}); wherein —R^{y1}, —R^{y6}, —R^{y6a} are independently selected from the group consisting of H and C₁₋₄ alkyl and wherein T is selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl.

Preferably, -L²- has a molecular weight in the range of from 14 g/mol to 750 g/mol.

Preferably, -L²- comprises a moiety selected from

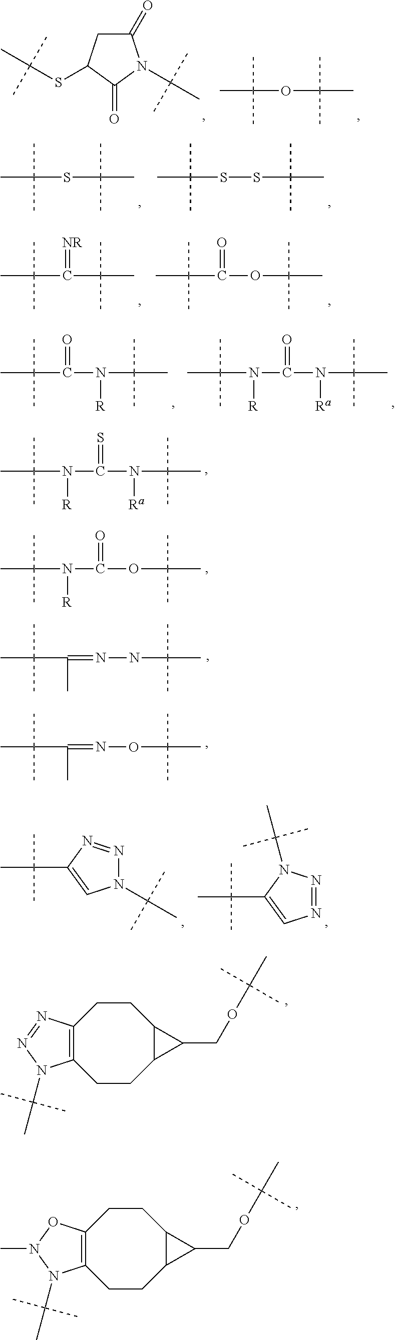

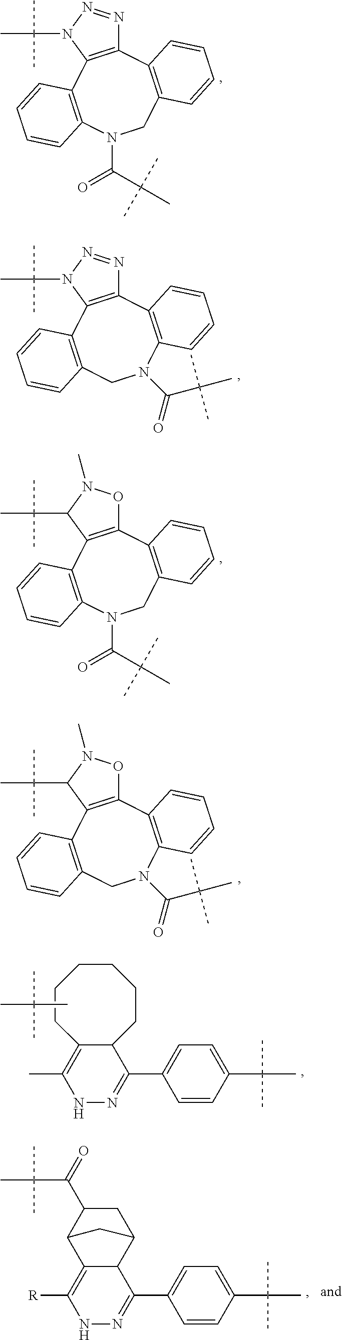

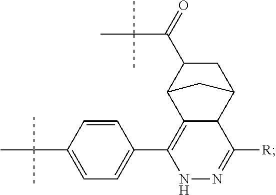

wherein

dashed lines indicate attachment to -L¹-, the remainder of -L²- or —Z, respectively; and

—R and —R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

In certain embodiments -L²- comprises a moiety

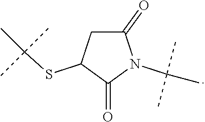

In certain embodiments -L²- comprises a moiety

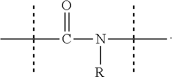

In certain embodiments Z is degradable. Irradiating degradable polymer carriers is particularly challenging, because the degradable bonds may be damaged or additional cross-links could be introduced during irradiation. Both reactions would alter the biophysical properties of the polymer and potentially impact its intended performance. In the context of the use for drug delivery purposes, this may result in changes in the safety or efficacy profile, or both. It was now surprisingly found that the degradation half-life after irradiation of such degradable Z varies by no more than 20% compared to corresponding non-irradiated Z.

In certain embodiments Z is a hydrogel.

In certain embodiments such hydrogel Z comprises a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(alkylene glycols), such as poly(ethylene glycols) and poly(propylene glycol), poly(ethylene oxides), poly(ethyl phosphates), poly(ethyloxazolines), poly(glycolic acids), poly(hydroxyethyl acrylates), poly(hy droxy ethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

In certain embodiments Z is a poly(alkylene glycol)-based or hyaluronic acid-based hydrogel.

In certain embodiments Z is a poly(propylene glycol)-based hydrogel.

In certain embodiments Z is a PEG-based hydrogel. Suitable hydrogels are known in the art. Examples are WO2006/003014, WO2011/012715 and WO2014/056926, which are herewith incorporated by reference.

In certain embodiments such PEG-based hydrogel comprises a plurality of backbone moieties that are crosslinked via crosslinker moieties -CL^{p}-. Optionally, there is a spacer moiety -SP¹- between a backbone moiety and a crosslinker moiety. In certain embodiments such spacer -SP¹- is defined as described above for -L²-.

In certain embodiments a backbone moiety has a molecular weight ranging from 1 kDa to 20 kDa.

In certain embodiments a backbone moiety is of formula (pA)

B*-(A-Hyp)ₓ (pA),
wherein
B* is a branching core,
A is a PEG-based polymer,
Hyp is a branched moiety,
x is an integer of from 3 to 16;
and wherein each backbone moiety is connected to one or more crosslinker moieties and to one or more moieties -L²-, which crosslinker moieties and moieties -L²- are connected to Hyp, either directly or through a spacer moiety.

In certain embodiments B* of formula (pA) is selected from the group consisting of polyalcohol moieties and polyamine moieties. In certain embodiments B* of formula (pA) is a polyalcohol moiety. In certain embodiments B* of formula (pA) is a polyamine moiety.

In certain embodiments the polyalcohol moieties for B* of formula (pA) are selected from the group consisting of a pentaerythritol moiety, tripentaerythritol moiety, hexaglycerine moiety, sucrose moiety, sorbitol moiety, fructose moiety, mannitol moiety and glucose moiety. In certain embodiments B* of formula (pA) is a pentaerythritol moiety, i.e. a moiety of formula

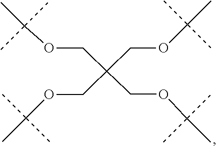

wherein dashed lines indicate attachment to -A-.

In certain embodiments the polyamine moieties for B* of formula (pA) is selected from the group consisting of an ornithine moiety, diaminobutyric acid moiety, trilysine moiety, tetralysine moiety, pentalysine moiety, hexalysine moiety, heptalysine moiety, octalysine moiety, nonalysine moiety, decalysine moiety, undecalysine moiety, dodecalysine moiety, tridecalysine moiety, tetradecalysine moiety and pentadecalysine moiety. In certain embodiments B* of formula (pA) is selected from the group consisting of an ornithine moiety, diaminobutyric acid moiety and a trilysine moiety.

A backbone moiety of formula (pA) may consist of the same or different PEG-based moieties -A- and each moiety -A- may be chosen independently. In certain embodiments all moieties -A- present in a backbone moiety of formula (pA) have the same structure. It is understood that the phrase “have the same structure” with regard to polymeric moieties, such as with regard to the PEG-based polymer -A-, means that the number of monomers of the polymer, such as the number of ethylene glycol monomers, may vary due to the polydisperse nature of polymers. In certain embodiments the number of monomer units does not vary by more than a factor of 2 between all moieties -A- of a hydrogel.

In certain embodiments each -A- of formula (pA) has a molecular weight ranging from 0.3 kDa to 40 kDa; e.g. from 0.4 to 30 kDa, from 0.4 to 25 kDa, from 0.4 to 20 kDa, from 0.4 to 15 kDa, from 0.4 to 10 kDa or from 0.4 to 5 kDa. In certain embodiments each -A- has a molecular weight from 0.4 to 5 kDa. In certain embodiments -A- has a molecular weight of about 0.5 kDa. In certain embodiments -A- has a molecular weight of about 1 kDa. In certain embodiments -A- has a molecular weight of about 2 kDa. In certain embodiments -A- has a molecular weight of about 3 kDa. In certain embodiments -A- has a molecular weight of about 5 kDa.

In certain embodiments -A- of formula (pA) is of formula (pB-i)

—(CH₂)ₙ₁(OCH₂CH₂)ₙX— (PB-i),
wherein
n1 is 1 or 2;
n is an integer ranging from 3 to 250, such as from 5 to 200, such as from 8 to 150 or from 10 to 100; and
X is a chemical bond or a linkage covalently linking A and Hyp.

In certain embodiments -A- of formula (pA) is of formula (pB-ii)

—(CH₂)ₙ₁(OCH₂CH₂)ₙ—(CH₂)ₙ₂X— (pB-ii),
wherein
n1 is 1 or 2;
n is an integer ranging from 3 to 250, such as from 5 to 200, such as from 8 to 150 or from 10 to 100;
n2 is 0 or 1; and
X is a chemical bond or a linkage covalently linking A and Hyp.

In certain embodiments -A- of formula (pA) is of formula (pB-i′)

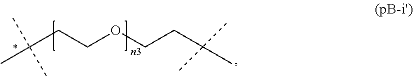

wherein
the dashed line marked with the asterisk indicates attachment to B*,
the unmarked dashed line indicates attachment to -Hyp; and
n3 is an integer ranging from 10 to 50.

In certain embodiments n3 of formula (pB-i′) is 25. In certain embodiments n3 of formula (pB-i′) is 26. In certain embodiments n3 of formula (pB-i′) is 27. In certain embodiments n3 of formula (pB-i′) is 28. In certain embodiments n3 of formula (pB-i′) is 29. In certain embodiments n3 of formula (pB-i′) is 30.

In certain embodiments a moiety B*-(A)₄ is of formula (pB-a)

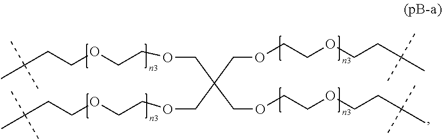

wherein
dashed lines indicate attachment to Hyp; and
each n3 is independently an integer selected from 10 to 50.

In certain embodiments n3 of formula (pB-a) is 25. In certain embodiments n3 of formula (pB-a) is 26. In certain embodiments n3 of formula (pB-a) is 27. In certain embodiments n3 of formula (pB-a) is 28. In certain embodiments n3 of formula (pB-a) is 29. In certain embodiments n3 of formula (pB-a) is 30.

A backbone moiety of formula (pA) may consist of the same or different dendritic moieties -Hyp and that each -Hyp can be chosen independently. In certain embodiments all moieties -Hyp present in a backbone moiety of formula (pA) have the same structure.

In certain embodiments each -Hyp of formula (pA) has a molecular weight ranging from 0.3 kDa to 5 kDa.

In certain embodiments -Hyp is selected from the group consisting of a moiety of formula (pHyp-i)

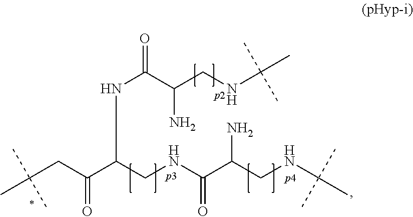

wherein
the dashed line marked with the asterisk indicates attachment to -A-,
the unmarked dashed lines indicate attachment to a spacer moiety -SP¹-, a crosslinker moiety -CL^{p}- or to -L²-; and
p2, p3 and p4 are identical or different and each is independently of the others an integer from 1 to 5;

a moiety of formula (pHyp-ii)

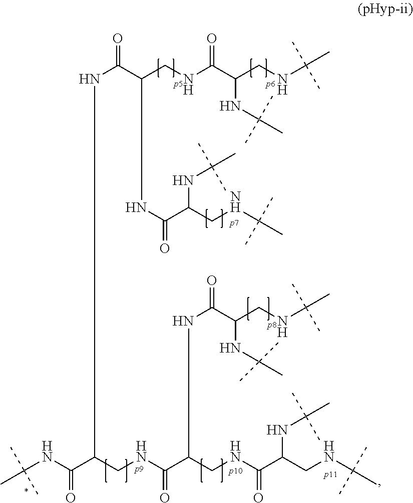

wherein
the dashed line marked with the asterisk indicates attachment to -A-,
the unmarked dashed lines indicate attachment to a spacer moiety -SP¹-, a crosslinker moiety -CL^{p}- or to -L²-; and
p5 to p11 are identical or different and each is independently of the others an integer from 1 to 5;

a moiety of formula (pHyp-iii)

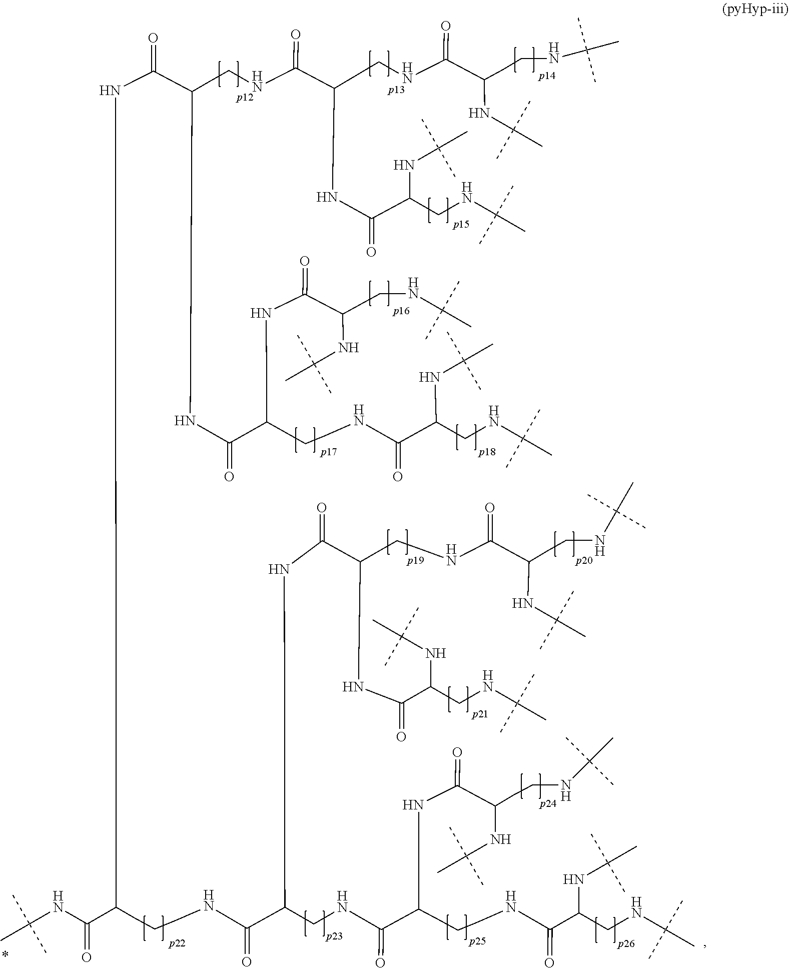

wherein
the dashed line marked with the asterisk indicates attachment to -A-,
the unmarked dashed lines indicate attachment to a spacer moiety -SP¹-, a crosslinker moiety -CL^{p}- or to -L²-; and
p12 to p26 are identical or different and each is independently of the others an integer from 1 to 5; and

a moiety of formula (pHyp-iv)

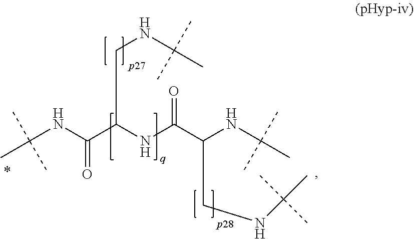

wherein
the dashed line marked with the asterisk indicates attachment to -A-,
the unmarked dashed lines indicate attachment to a spacer moiety -SP¹-, a crosslinker moiety -CL^{p}- or to -L²-;
p27 and p28 are identical or different and each is independently of the other an integer from 1 to 5; and
q is an integer from 1 to 8;

wherein the moieties (pHyp-i) to (pHyp-iv) may at each chiral center be in either R- or S-configuration.

In certain embodiments all chiral centers of a moiety (pHyp-i), (pHyp-ii), (pHyp-iii) or (pHyp-iv) are in the same configuration. In certain embodiments all chiral centers of a moiety (pHyp-i), (pHyp-ii), (pHyp-iii) or (pHyp-iv) are in R-configuration. In certain embodiments all chiral centers of a moiety (pHyp-i), (pHyp-ii), (pHyp-iii) or (pHyp-iv) are in S-configuration.

In certain embodiments p2, p3 and p4 of formula (pHyp-i) are 4. In certain embodiments p5 to p11 of formula (pHyp-ii) are 4. In certain embodiments p12 to p26 of formula (pHyp-iii) are 4. In certain embodiments q of formula (pHyp-iv) is 2 or 6. In certain embodiments q of formula (pHyp-iv) q is 6. In certain embodiments p27 and p28 of formula (pHyp-iv) are 4.

In certain embodiments -Hyp of formula (pA) comprises a branched polypeptide moiety.

In certain embodiments -Hyp of formula (pA) comprises a lysine moiety. In certain embodiments each -Hyp of formula (pA) is independently selected from the group consisting of a trilysine moiety, tetralysine moiety, pentalysine moiety, hexalysine moiety, heptalysine moiety, octalysine moiety, nonalysine moiety, decalysine moiety, undecalysine moiety, dodecalysine moiety, tridecalysine moiety, tetradecalysine moiety, pentadecalysine moiety, hexadecalysine moiety, heptadecalysine moiety, octadecalysine moiety and nonadecalysine moiety.

In certain embodiments -Hyp comprises 3 lysine moieties. In certain embodiments -Hyp comprises 7 lysine moieties. In certain embodiments -Hyp comprises 15 lysine moieties. In certain embodiments -Hyp comprises heptalysinyl.

In certain embodiments x of formula (pA) is 3. In certain embodiments x of formula (pA) is 4.

In certain embodiments x of formula (pA) is 6. In certain embodiments x of formula (pA) is 8.

In certain embodiments the backbone moiety is of formula (pC)

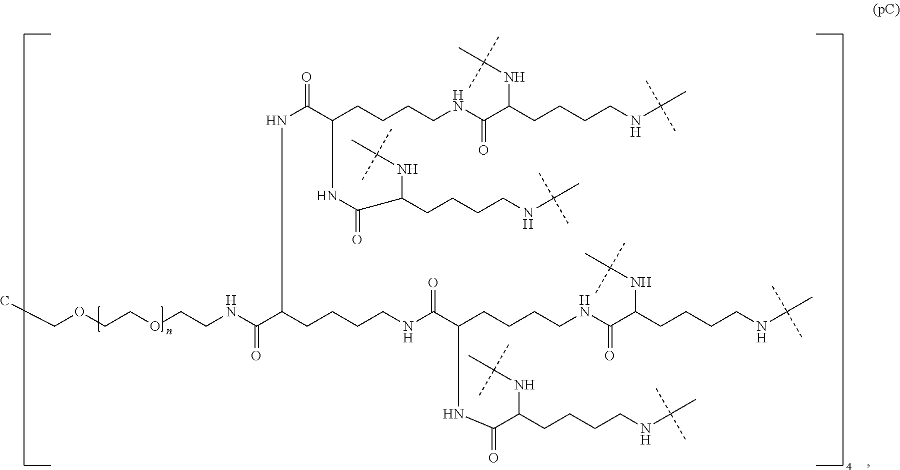

wherein
dashed lines indicate attachment to a spacer moiety -SP¹-, a crosslinker moiety -CL^{p}- or to -L²-; and
n ranges from 10 to 40.

In certain embodiments n of formula (pC) is about 28.

In certain embodiments there is no spacer moiety -SP¹- between a backbone moiety and a crosslinker moiety -CL^{p}-, i.e. -CL^{p}- is directly linked to -Hyp.

The crosslinker -CL^{p}- of the PEG-based hydrogel is in certain embodiments poly(alkylene glycol) (PAG)-based. In certain embodiments the crosslinker is poly(propylene glycol)-based. In certain embodiments the crosslinker -CL^{p}- is PEG-based.

In certain embodiments such PAG-based crosslinker moiety -CL^{p}- is of formula (pD)

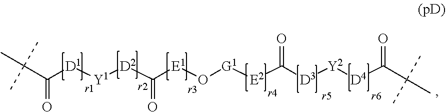

wherein
dashed lines indicate attachment to a backbone moiety or to a spacer moiety -SP¹-; —Y¹— is of formula

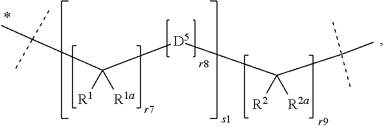

wherein the dashed line marked with the asterisk indicates attachment to -D¹- and the unmarked dashed line indicates attachment to -D²-;
—Y²- is of formula

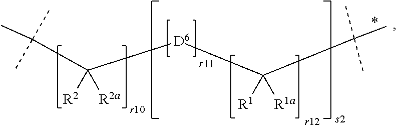

wherein the dashed line marked with the asterisk indicates attachment to -D⁴- and the unmarked dashed line indicates attachment to -D³-;
-E¹- is of formula

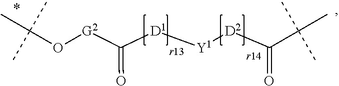

wherein the dashed line marked with the asterisk indicates attachment to —(C═O)— and the unmarked dashed line indicates attachment to —O—;
-E²- is of formula

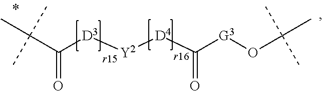

wherein the dashed line marked with the asterisk indicates attachment to -G¹- and the unmarked dashed line indicates attachment to —(C═O)—;
-G¹- is of formula

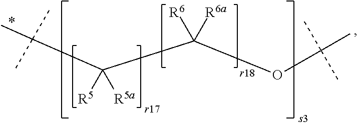

wherein the dashed line marked with the asterisk indicates attachment to —O— and the unmarked dashed line indicates attachment to -E²-;
-G²- is of formula

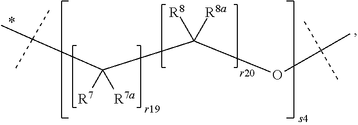

wherein the dashed line marked with the asterisk indicates attachment to —O— and the unmarked dashed line indicates attachment to —(C═O)—;
-G³- is of formula

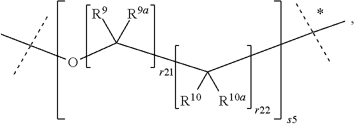

wherein the dashed line marked with the asterisk indicates attachment to —O— and the unmarked dashed line indicates attachment to —(C═O)—;
-D¹-, -D²-, -D³-, -D⁴-, -D⁵- and -D⁶- are identical or different and each is independently of the others selected from the group comprising —O—, —NR¹¹, N⁺R¹²R^{12a}—, —S—, —(S═O)—, —(S(O)₂)—, —C(O)—, —P(O)R¹³—, —P(O)(OR¹³) and —CR¹⁴R^{14a}—;
—R¹, —R^{1a}, —R², —R^{2a}, —R³, —R^{3a}, —R⁴, —R^{4a}, —R⁵, —R^{5a}, —R⁶, —R^{6a}, —R⁷, —R^{7a}, —R⁸, —R^{8a}, —R⁹, —R^{9a}, —R¹⁰, —R^{10a}, —R¹¹, —R¹², —R^{12a}, —R¹³, —R¹⁴ and —R^{14a} are identical or different and each is independently of the others selected from the group consisting of —H and C₁₋₆ alkyl;
optionally, one or more of the pairs —R¹/—R^{1a}, —R²/—R^{2a}, —R³/—R^{3a}, —R⁴/—R^{4a}, —R¹/—R², —R³/—R⁴, —R^{1a}/—R^{2a}, —R^{3a}/—R^{4a}, —R¹²/—R^{12a}, and —R¹⁴/—R^{14a} form a chemical bond or are joined together with the atom to which they are attached to form a C₃₋₈ cycloalkyl or to form a ring A or are joined together with the atom to which they are attached to form a 4- to 7-membered heterocyclyl or 8- to 11-membered heterobicyclyl or adamantyl;
A is selected from the group consisting of phenyl, naphthyl, indenyl, indanyl and tetralinyl;
r1, r2, r5, r6, r13, r14, r15 and r16 are independently 0 or 1;
r3, r4, r7, r8, r9, r10, r11, r12 are independently 0, 1, 2, 3, or 4;
r17, r18, r19, r20, r21 and r22 are independently 1, 2, 3, 4, 5, 6, 7, 8, 9 or 10;
s1, s2, s4, s5 are independently 1, 2, 3, 4, 5 or 6; and
s3 ranges from 1 to 900.

In certain embodiments s3 ranges from 1 to 500. In certain embodiments s3 ranges from 1 to 200.

In certain embodiments r1 of formula (pD) is 0. In certain embodiments r1 of formula (pD) is 1. In certain embodiments r2 of formula (pD) is 0. In certain embodiments r2 of formula (pD) is 1. In certain embodiments r5 of formula (pD) is 0. In certain embodiments r5 of formula (pD) is 1.

In certain embodiments r1, r2, r5 and r6 of formula (pD) are 0.

In certain embodiments r6 of formula (pD) is 0. In certain embodiments r6 of formula (pD) is 1. In certain embodiments r13 of formula (pD) is 0. In certain embodiments r13 of formula (pD) is 1. In certain embodiments r14 of formula (pD) is 0. In certain embodiments r14 of formula (pD) is 1. In certain embodiments r15 of formula (pD) is 0. In certain embodiments r15 of formula (pD) is 1. In certain embodiments r16 of formula (pD) is 0. In certain embodiments r16 of formula (pD) is 1.

In certain embodiments r3 of formula (pD) is 1. In certain embodiments r3 of formula (pD) is 2. In certain embodiments r4 of formula (pD) is 1. In certain embodiments r4 of formula (pD) is 2. In certain embodiments r3 and r4 of formula (pD) are both 1. In certain embodiments r3 and r4 of formula (pD) are both 2. In certain embodiments r3 and r4 of formula (pD) are both 3.

In certain embodiments r7 of formula (pD) is 0. In certain embodiments r7 of formula (pD) is 1. In certain embodiments r7 of formula (pD) is 2. In certain embodiments r8 of formula (pD) is 0. In certain embodiments r8 of formula (pD) is 1. In certain embodiments r8 of formula (pD) is 2. In certain embodiments r9 of formula (pD) is 0. In certain embodiments r9 of formula (pD) is 1. In certain embodiments r9 of formula (pD) is 2. In certain embodiments r10 of formula (pD) is 0. In certain embodiments r10 of formula (pD) is 1. In certain embodiments r10 of formula (pD) is 2. In certain embodiments r11 of formula (pD) is 0. In certain embodiments r11 of formula (pD) is 1. In certain embodiments r1 l of formula (pD) is 2. In certain embodiments r12 of formula (pD) is 0. In certain embodiments r12 of formula (pD) is 1. In certain embodiments r12 of formula (pD) is 2.

In certain embodiments r17 of formula (pD) is 1. In certain embodiments r18 of formula (pD) is 1. In certain embodiments r19 of formula (pD) is 1. In certain embodiments r20 of formula (pD) is 1. In certain embodiments r21 of formula (pD) is 1.

In certain embodiments s1 of formula (pD) is 1. In certain embodiments s1 of formula (pD) is 2. In certain embodiments s2 of formula (pD) is 1. In certain embodiments s2 of formula (pD) is 2. In certain embodiments s4 of formula (pD) is 1. In certain embodiments s4 of formula (pD) is 2.

In certain embodiments s3 of formula (pD) ranges from 5 to 500. In certain embodiments s3 of formula (pD) ranges from 10 to 250. In certain embodiments s3 of formula (pD) ranges from 12 to 150. In certain embodiments s3 of formula (pD) ranges from 15 to 100. In certain embodiments s3 of formula (pD) ranges from 18 to 75. In certain embodiments s3 of formula (pD) ranges from 20 to 50.

In certain embodiments —R¹ of formula (pD) is —H. In certain embodiments —R¹ of formula (pD) is methyl. In certain embodiments —R¹ of formula (pD) is ethyl. In certain embodiments —R^{1a} of formula (pD) is —H. In certain embodiments —R^{1a} of formula (pD) is methyl. In certain embodiments —R^{1a} of formula (pD) is ethyl. In certain embodiments —R² of formula (pD) is —H. In certain embodiments —R² of formula (pD) is methyl. In certain embodiments —R² of formula (pD) is ethyl. In certain embodiments —R^{2a} of formula (pD) is —H. In certain embodiments —R^{2a} of formula (pD) is methyl. In certain embodiments —R^{2a} of formula (pD) is ethyl. In certain embodiments —R³ of formula (pD) is —H. In certain embodiments —R³ of formula (pD) is methyl. In certain embodiments —R³ of formula (pD) is ethyl. In certain embodiments —R^{3a} of formula (pD) is —H. In certain embodiments —R^{3a} of formula (pD) is methyl. In certain embodiments —R^{3a} of formula (pD) is ethyl. In certain embodiments —R⁴ of formula (pD) is —H. In certain embodiments —R⁴ of formula (pD) is methyl. In certain embodiments —R⁴ of formula (pD) is methyl. In certain embodiments —R^{4a} of formula (pD) is —H. In certain embodiments —R^{4a} of formula (pD) is methyl. In certain embodiments —R^{4a} of formula (pD) is ethyl. In certain embodiments —R⁵ of formula (pD) is —H. In certain embodiments —R⁵ of formula (pD) is methyl. In certain embodiments —R⁵ of formula (pD) is ethyl. In certain embodiments —R^{5a} of formula (pD) is —H. In certain embodiments —R^{5a} of formula (pD) is methyl. In certain embodiments —R^{5a} of formula (pD) is ethyl. In certain embodiments —R⁶ of formula (pD) is —H. In certain embodiments —R⁶ of formula (pD) is methyl. In certain embodiments —R⁶ of formula (pD) is ethyl. In certain embodiments —R^{6a} of formula (pD) is —H. In certain embodiments —R^{6a} of formula (pD) is methyl. In certain embodiments —R^{6a} of formula (pD) is ethyl. In certain embodiments —R⁷ of formula (pD) is —H. In certain embodiments —R⁷ of formula (pD) is methyl. In certain embodiments —R⁷ of formula (pD) is ethyl. In certain embodiments —R⁸ of formula (pD) is —H. In certain embodiments —R⁸ of formula (pD) is methyl. In certain embodiments —R⁸ of formula (pD) is ethyl. In certain embodiments —R^{8a} of formula (pD) is —H. In certain embodiments —R^{8a} of formula (pD) is methyl. In certain embodiments —R^{8a} of formula (pD) is ethyl. In certain embodiments —R⁹ of formula (pD) is —H. In certain embodiments —R⁹ of formula (pD) is methyl. In certain embodiments —R⁹ of formula (pD) is ethyl. In certain embodiments —R^{9a} of formula (pD) is —H. In certain embodiments —R^{9a} of formula (pD) is methyl. In certain embodiments —R^{9a} of formula (pD) is ethyl. In certain embodiments —R¹⁰ of formula (pD) is —H. In certain embodiments —R¹⁰ of formula (pD) is methyl. In certain embodiments —R¹⁰ of formula (pD) is ethyl. In certain embodiments —R^{10a} of formula (pD) is —H. In certain embodiments —R^{10a} of formula (pD) is methyl. In certain embodiments —R^{10a} of formula (pD) is ethyl. In certain embodiments —R¹¹ of formula (pD) is —H. In certain embodiments —R¹¹ of formula (pD) is methyl. In certain embodiments —R¹¹ of formula (pD) is ethyl. In certain embodiments —R¹² of formula (pD) is —H. In certain embodiments —R¹² of formula (pD) is methyl. In certain embodiments —R¹² of formula (pD) is ethyl. In certain embodiments —R^{12a} of formula (pD) is —H. In certain embodiments —R^{12a} of formula (pD) is methyl. In certain embodiments —R^{12a} of formula (pD) is ethyl. In certain embodiments —R¹³ of formula (pD) is —H. In certain embodiments —R¹³ of formula (pD) is methyl. In certain embodiments —R¹³ of formula (pD) is ethyl. In certain embodiments —R¹⁴ of formula (pD) is —H. In certain embodiments —R¹⁴ of formula (pD) is methyl. In certain embodiments —R¹⁴ of formula (pD) is ethyl. In certain embodiments —R^{14a} of formula (pD) is —H. In certain embodiments —R^{14a} of formula (pD) is methyl. In certain embodiments —R^{14a} of formula (pD) is ethyl.

In certain embodiments -D¹- of formula (pD) is —O—. In certain embodiments -D¹- of formula (pD) is —NR¹¹—. In certain embodiments -D¹- of formula (pD) is —N⁺R¹²R^{12a}—. In certain embodiments -D¹- of formula (pD) is —S—. In certain embodiments -D¹- of formula (pD) is —(S═O). In certain embodiments -D¹- of formula (pD) is —(S(O)₂)—. In certain embodiments -D¹- of formula (pD) is —C(O)—. In certain embodiments -D¹- of formula (pD) is —P(O)R¹³—. In certain embodiments -D¹- of formula (pD) is —P(O)(OR¹³)—. In certain embodiments -D¹- of formula (pD) is —CR¹⁴R^{14a}—.

In certain embodiments -D²- of formula (pD) is —O—. In certain embodiments -D²- of formula (pD) is —NR¹¹—. In certain embodiments -D²- of formula (pD) is —N⁺R¹²R^{12a}—. In certain embodiments -D²- of formula (pD) is —S—. In certain embodiments -D²- of formula (pD) is —(S═O). In certain embodiments -D²- of formula (pD) is —(S(O)₂)—. In certain embodiments -D²- of formula (pD) is —C(O)—. In certain embodiments -D²- of formula (pD) is —P(O)R¹³—. In certain embodiments -D²- of formula (pD) is —P(O)(OR¹³)—. In certain embodiments -D²- of formula (pD) is —CR¹⁴R^{14a}—.

In certain embodiments -D³- of formula (pD) is —O—. In certain embodiments -D³- of formula (pD) is —NR¹¹—. In certain embodiments -D³- of formula (pD) is —N⁺R¹²R^{12a}—. In certain embodiments -D³- of formula (pD) is —S—. In certain embodiments -D³- of formula (pD) is —(S═O). In certain embodiments -D³- of formula (pD) is —(S(O)₂)—. In certain embodiments -D³- of formula (pD) is —C(O)—. In certain embodiments -D³- of formula (pD) is —P(O)R¹³—. In certain embodiments -D³- of formula (pD) is —P(O)(OR¹³)—. In certain embodiments -D³- of formula (pD) is —CR¹⁴R^{14a}—.

In certain embodiments -D⁴- of formula (pD) is —O—. In certain embodiments -D⁴- of formula (pD) is —NR¹¹—. In certain embodiments -D⁴- of formula (pD) is —N⁺R¹²R^{12a}—. In certain embodiments -D⁴- of formula (pD) is —S—. In certain embodiments -D⁴- of formula (pD) is —(S═O). In certain embodiments -D⁴- of formula (pD) is —(S(O)₂)—. In certain embodiments -D⁴- of formula (pD) is —C(O)—. In certain embodiments -D⁴- of formula (pD) is —P(O)R¹³—. In certain embodiments -D⁴- of formula (pD) is —P(O)(OR¹³)—. In certain embodiments -D⁴- of formula (pD) is —CR¹⁴R^{14a}—.

In certain embodiments -D⁵- of formula (pD) is —O—. In certain embodiments -D⁵- of formula (pD) is —NR¹¹—. In certain embodiments -D⁵- of formula (pD) is —N⁺R¹²R^{12a}—. In certain embodiments -D⁵- of formula (pD) is —S—. In certain embodiments -D⁵- of formula (pD) is —(S═O)—. In certain embodiments -D⁵- of formula (pD) is —(S(O)₂)—. In certain embodiments -D⁵- of formula (pD) is —C(O)—. In certain embodiments -D⁵- of formula (pD) is —P(O)R¹³—. In certain embodiments -D⁵- of formula (pD) is —P(O)(OR¹³)—. In certain embodiments -D⁵- of formula (pD) is —CR¹⁴R^{14a}—.

In certain embodiments -D⁶- of formula (pD) is —O—. In certain embodiments -D⁶- of formula (pD) is —NR¹¹—. In certain embodiments -D⁶- of formula (pD) is —N⁺R¹²R^{12a}—. In certain embodiments -D⁶- of formula (pD) is —S—. In certain embodiments -D⁶- of formula (pD) is —(S═O). In certain embodiments -D⁶- of formula (pD) is —(S(O)₂)—. In certain embodiments -D⁶- of formula (pD) is —C(O)—. In certain embodiments -D⁶- of formula (pD) is —P(O)R¹³—. In certain embodiments -D⁶- of formula (pD) is —P(O)(OR¹³)—. In certain embodiments -D⁶- of formula (pD) is —CR¹⁴R^{14a}—.

In one embodiment -CL^{p}- is of formula (pE)

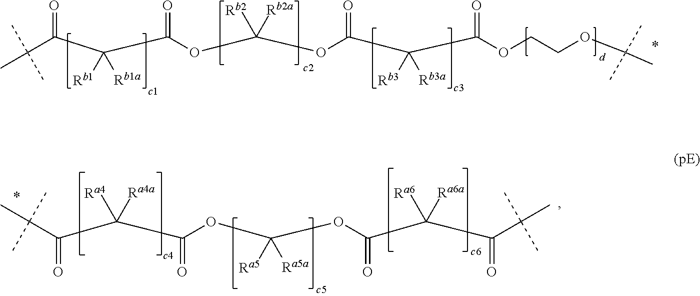

wherein
dashed lines marked with an asterisk indicate the connection point between the upper and the lower substructure,
unmarked dashed lines indicate attachment to a backbone moiety or to a spacer moiety -SP¹-;
—R^{b1}, —R^{b1a}, —R^{b2}, —R^{b2a}, —R^{b3}, —R^{b3a}, —R^{a4}, —R^{a4a}, —R^{a5}, —R^{a5a}, —R^{a6} and —R^{a6} are independently are independently selected from the group consisting of —H and C₁₋₆ alkyl;
c1, c2, c3, c4, c5 and c6 are independently selected from the group consisting of 1, 2, 3, 4, 5 and 6;
d is an integer ranging from 2 to 250.

In certain embodiments d of formula (pE) ranges from 3 to 200. In certain embodiments d of formula (pE) ranges from 4 to 150. In certain embodiments d of formula (pE) ranges from 5 to 100. In certain embodiments d of formula (pE) ranges from 10 to 50. In certain embodiments d of formula (pE) ranges from 15 to 30. In certain embodiments d of formula (pE) is about 23.

In certain embodiments —R^{b1} and —R^{b1a} of formula (pE) are —H. In certain embodiments —R^{b2} and —R^{b2a} of formula (pE) are —H. In certain embodiments —R^{b3} and —R^{b3a} of formula (pE) are —H. In certain embodiments —R^{a4} and —R^{a4a} of formula (pE) are —H. In certain embodiments —R^{a5} and —R^{a5a} of formula (pE) are —H. In certain embodiments —R^{a6} and —R^{a6a} of formula (pE) are —H. In certain embodiments —R^{b1}, —R^{b1a}, —R^{b2}, —R^{b2a}, —R^{b3}, —R^{b3a}, —R^{a4}, —R^{a4a}, —R^{a5}, —R^{a5a}, —R^{a6} and —R^{a6a} of formula (pE) are all —H.

In certain embodiments c1 of formula (pE) is 1. In certain embodiments c1 of formula (pE) is 2. In certain embodiments c1 of formula (pE) is 3. In certain embodiments c1 of formula (pE) is 4. In certain embodiments c1 of formula (pE) is 5. In certain embodiments c1 of formula (pE) is 6.

In certain embodiments c2 of formula (pE) is 1. In certain embodiments c2 of formula (pE) is 2. In certain embodiments c2 of formula (pE) is 3. In certain embodiments c2 of formula (pE) is 4. In certain embodiments c2 of formula (pE) is 5. In certain embodiments c2 of formula (pE) is 6.

In certain embodiments c3 of formula (pE) is 1. In certain embodiments c3 of formula (pE) is 2. In certain embodiments c3 of formula (pE) is 3. In certain embodiments c3 of formula (pE) is 4. In certain embodiments c3 of formula (pE) is 5. In certain embodiments c3 of formula (pE) is 6.

In certain embodiments c4 of formula (pE) is 1. In certain embodiments c4 of formula (pE) is 2. In certain embodiments c4 of formula (pE) is 3. In certain embodiments c4 of formula (pE) is 4. In certain embodiments c4 of formula (pE) is 5. In certain embodiments c4 of formula (pE) is 6.

In certain embodiments c5 of formula (pE) is 1. In certain embodiments c5 of formula (pE) is 2. In certain embodiments c5 of formula (pE) is 3. In certain embodiments c5 of formula (pE) is 4. In certain embodiments c5 of formula (pE) is 5. In certain embodiments c5 of formula (pE) is 6.

In certain embodiments c6 of formula (pE) is 1. In certain embodiments c6 of formula (pE) is 2. In certain embodiments c6 of formula (pE) is 3. In certain embodiments c6 of formula (pE) is 4. In certain embodiments c6 of formula (pE) is 5. In certain embodiments c6 of formula (pE) is 6.

In certain embodiments a crosslinker moiety -CL^{p}- is of formula (pE-i)

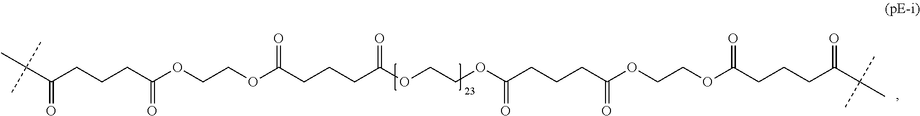

wherein

dashed lines indicate attachment to a backbone moiety or to a spacer moiety -SP¹-.

In certain embodiments —Z is a hyaluronic acid-based hydrogel. Such hyaluronic acid-based hydrogels are known in the art, such as for example from WO2018/175788, which is incorporated herewith by reference.

If —Z is a hyaluronic acid-based hydrogel, a conjugate of the present invention is in certain embodiments a conjugate comprising crosslinked hyaluronic acid strands to which a plurality of drug moieties is covalently and reversibly conjugated, wherein the conjugate comprises a plurality of connected units selected from the group consisting of

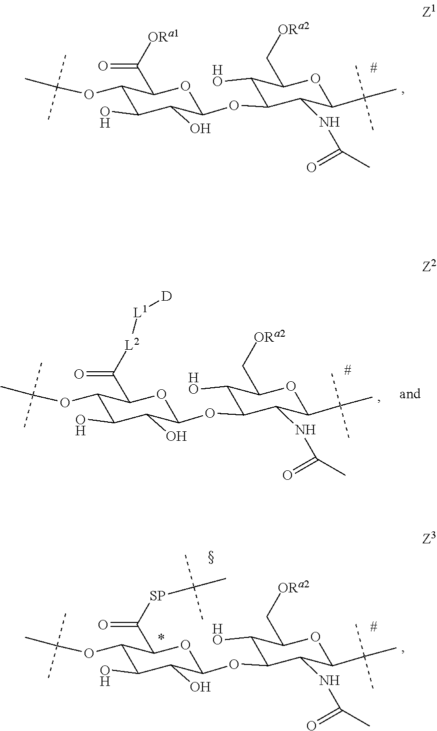

wherein
an unmarked dashed line indicates a point of attachment to an adjacent unit at a dashed line marked with # or to a hydrogen;
a dashed line marked with # indicates a point of attachment to an adjacent unit at an unmarked dashed line or to a hydroxyl;
a dashed line marked with § indicates a point of connection between at least two units Z³ via a moiety -CL-;
each -D, -L¹-, and -L² are used as defined above;
each -CL- is independently a moiety connecting at least two units Z³ and wherein there is at least one degradable bond in the direct connection between any two carbon atoms marked with the*connected by a moiety -CL-;
each -SP- is independently absent or a spacer moiety;
each —R^{a1} is independently selected from the group consisting of —H, C₁₋₄ alkyl, an ammonium ion, a tetrabutylammonium ion, a cetyl methylammonium ion, an alkali metal ion and an alkaline earth metal ion;
each —R^{a2} is independently selected from the group consisting of —H and C₁₋₁₀ alkyl;
wherein
all units Z¹ present in the conjugate may be the same or different;
all units Z² present in the conjugate may be the same or different;
all units Z³ present in the conjugate may be the same or different;
at least one unit Z³ is present per hyaluronic acid strand which is connected to at least one unit Z³ on a different hyaluronic acid strand; and
the conjugate comprises at least one moiety -L²-L¹-D.

The presence of at least one degradable bond between the carbon atom marked with the*of a first moiety Z³ and the direct connection to the carbon atom marked with the*of a second moiety Z³ ensures that after cleavage of all such degradable bonds the hyaluronic acid strands present in said conjugate are no longer crosslinked, which allows clearance of the hyaluronic acid network

It is understood that in case a degradable bond is located in a ring structure present in the direct connection of the carbon atom marked with the*of a first moiety Z³ and the carbon atom marked with the * of a second moiety Z³ such degradable bond is not sufficient to allow complete cleavage and accordingly one or more additional degradable bonds are present in the direct connection of the carbon atom marked with the * of a first moiety Z³ and the carbon atom marked with the * of a second moiety Z³.

It is understood that the phrase “a dashed line marked with § indicates a point of connection between at least two units Z³ via a moiety -CL-” refers to the following structure

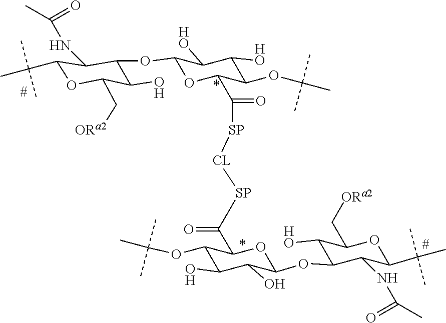

if -CL- is for example connected to two units Z³, which two moieties Z³ are connected at the position indicated with § via a moiety -CL-.

It is understood that no three-dimensionally crosslinked hydrogel can be formed if all hyaluronic acid strands of the present conjugate comprise only one unit Z³, which is connected to only one unit Z³ on a different hyaluronic acid strand. However, if a first unit Z³ is connected to more than one unit Z³ on a different strand, i.e. if -CL- is branched, such first unit Z³ may be crosslinked to two or more other units Z³ on two or more different hyaluronic acid strands. Accordingly, the number of units Z³ per hyaluronic acid strand required for a crosslinked hyaluronic acid hydrogel depends on the degree of branching of -CL-. In certain embodiments at least 30% of all hyaluronic acid strands present in the conjugate are connected to at least two other hyaluronic acid strands. It is understood that it is sufficient if the remaining hyaluronic acid strands are connected to only one other hyaluronic acid strand.

It is understood that such hydrogel also comprises partly reacted or unreacted units and that the presence of such moieties cannot be avoided. In certain embodiments the sum of such partly reacted or unreacted units is no more than 25% of the total number of units present in the conjugate, such as no more than 20%, such as no more than 15% or such as no more than 10%.

Furthermore, it is understood that in addition to units Z¹, Z² and Z³, partly reacted and unreacted units a conjugate may also comprise units that are the result of cleavage of the reversible bond between -D and -L¹- or of one or more of the degradable bonds present in the direct connection between any two carbon atoms marked with the * connected by a moiety -CL-, i.e. units resulting from degradation of the conjugate.

In certain embodiments each strand present in the conjugates of the present invention comprises at least 20 units, such as from 20 to 2500 units, from 25 to 2200 units, from 50 to 2000 units, from 75 to 100 units, from 75 to 100 units, from 80 to 560 units, from 100 to 250 units, from 200 to 800 units, from 20 to 1000, from 60 to 1000, from 60 to 400 or from 200 to 600 units.

In certain embodiments the moieties -CL- present in the conjugates of the present invention have different structures. In certain embodiments the moieties -CL- present in the conjugates of the present invention have the same structure.

In general, any moiety that connects at least two other moieties is suitable for use as a moiety -CL-, which may also be referred to as a “crosslinker moiety”.

The at least two units Z³ that are connected via a moiety -CL- may either be located on the same hyaluronic acid strand or on different hyaluronic acid strands.

The moiety -CL- may be linear or branched. In certain embodiments -CL- is linear. In certain embodiments -CL- is branched.

In certain embodiments -CL- connects two units Z³. In certain embodiments -CL- connects three units Z³. In certain embodiments -CL- connects four units Z³. In certain embodiments -CL- connects five units Z³. In certain embodiments -CL- connects six units Z³. In certain embodiments -CL- connects seven units Z³. In certain embodiments -CL- connects eight units Z³. In certain embodiments -CL- connects nine units Z³.

If -CL- connects two units Z³-CL- may be linear or branched. If -CL- connects more than two units Z³-CL- is branched.

A branched moiety -CL- comprises at least one branching point from which at least three branches extend, which branches may also be referred to as “arms”. Such branching point may be selected from the group consisting of

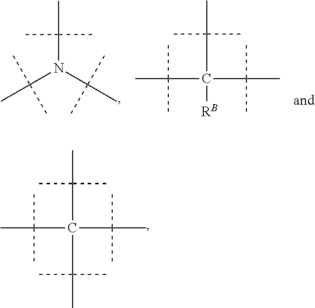

wherein
dashed lines indicate attachment to an arm; and
—R^{B} is selected from the group consisting of —H, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R^{B1}, which are the same or different, and wherein C₁₋₆ alkyl, C₂₋₆ alkenyl and C₂₋₆ alkynyl are optionally interrupted with —C(O)O—, —O—, —C(O)—, —C(O)N(R^{B2})—, —S(O)₂N(R^{B2})—, —S(O)N(R^{B2})—, —S(O)₂—, —S(O)—, —N(R^{B2})S(O)₂N(R^{B2a})—, —S—, —N(R^{B2})—, —OC(OR^{B2})(R^{B2a})—, —N(R^{B2})C(O)N(R^{B2a})—, and —OC(O)N(R^{B2})—; wherein —R^{B1}, —R^{B2} and —R^{B2a} are selected from —H, C₁₋₆ alkyl, C₂₋₆ alkenyl and C₂₋₆ alkynyl.

In certain embodiments —R^{B} is selected from the group consisting of —H, methyl and ethyl.

A branched moiety -CL- may comprise a plurality of branching points, such as 1, 2, 3, 4, 5, 6, 7 or more branching points, which may be the same or different.

If a moiety -CL- connects three units Z³, such moiety -CL- comprises at least one branching point from which at least three arms extend.

If a moiety -CL- connects four units Z³, such moiety -CL- may comprise one branching point from which four arms extend. However, alternative geometries are possible, such as at least two branching points from which at least three arms each extend. The larger the number of connected units Z³, the larger the number of possible geometries is.

In a first embodiment at least 70%, such as at least 75%, such as at least 80%, such as at least 85%, such as at least 90% or such as at least 95% of the number of hyaluronic acid strands of the conjugate of the present invention comprise at least one moiety Z² and at least one moiety Z³. In such embodiment units Z² and Z³ can be found in essentially all hyaluronic acid strands present in the conjugates of the present invention.

Accordingly, a conjugate of this first embodiment comprises crosslinked hyaluronic acid strands to which a plurality of drug moieties are covalently and reversibly conjugated, wherein the conjugate comprises a plurality of connected units selected from the group consisting of

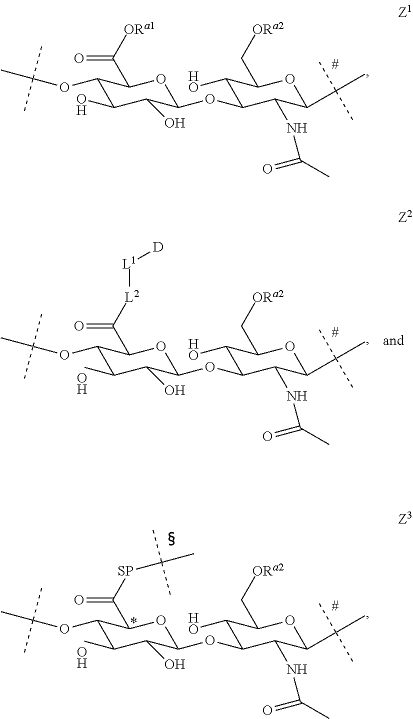

wherein
an unmarked dashed line indicates a point of attachment to an adjacent unit at a dashed line marked with # or to a hydrogen;
a dashed line marked with # indicates a point of attachment to an adjacent unit at an unmarked dashed line or to a hydroxyl;
a dashed line marked with § indicates a point of connection between at least two units Z³ via a moiety -CL-;
-D, -L¹-, -L²-, are used as defined above;
wherein
all units Z¹ present in the conjugate may be the same or different;
all units Z² present in the conjugate may be the same or different;
all units Z³ present in the conjugate may be the same or different;
the number of Z¹ units ranges from 1% to 98% of the total number of units present in the conjugate;
the number of Z² units ranges from 1% to 98% of the total number of units present in the conjugate, provided at least one unit Z² is present in the conjugate;
the number of Z³ units ranges from 1% to 97% of the total number of units present in the conjugate, provided that at least one unit Z³ is present per strand; and
wherein at least 70% of all hyaluronic acid strands comprise at least one moiety Z² and at least one moiety Z³.

In a conjugate according to this first embodiment the number of units Z² ranges from 1 to 70% of all units present in the conjugate, such as from 2 to 15%, from 2 to 10%, from 16 to 39, from 40 to 65%, or from 50 to 60% of all units present in the conjugate.

In a conjugate according to this first embodiment the number of units Z³ ranges from 1 to 30% of all units present in the conjugate, such as from 2 to 5%, from 5 to 20%, from 10 to 18%, or from 14 to 18% of all units present in the conjugate.

In a conjugate according to this first embodiment the number of units Z¹ ranges from 10 to 98% of all units present in the conjugate, such as from 20 to 40%, such as from 25 to 35%, such as from 41 to 95%, such as from 45 to 90%, such as from 50 to 70% of all units present in the conjugate.

Each degradable bond present in the direct connection between any two carbon atoms marked with the * connected by a moiety -CL- may be different or all such degradable bonds present in the conjugate may be the same.

Each direct connection between two carbon atoms marked with the * connected by a moiety -CL- may have the same or a different number of degradable bonds.

In certain embodiments the number of degradable bonds present in the conjugate of the present invention between all combinations of two carbon atoms marked with the * connected by a moiety -CL- is the same and all such degradable bonds have the same structure.

In the first embodiment the at least one degradable bond present in the direct connection between any two carbon atoms marked with the * connected by a moiety -CL- may be selected from the group consisting of ester, carbonate, sulfate, phosphate bonds, carbamate and amide bonds. It is understood that carbamates and amides are not reversible per se, and that in this context neighboring groups render these bonds reversible. In certain embodiments there is one degradable bond selected from the group consisting of ester, carbonate, sulfate, phosphate bonds, carbamate and amide bonds in the direct connection between any two carbon atoms marked with the * connected by a moiety -CL-. In certain embodiments there are two degradable bonds selected from the group consisting of ester, carbonate, sulfate, phosphate bonds, carbamate and amide bonds in the direct connection between any two carbon atoms marked with the * connected by a moiety -CL-, which degradable bonds may be the same or different. In certain embodiments there are three degradable bonds selected from the group consisting of ester, carbonate, sulfate, phosphate bonds, carbamate and amide bonds in the direct connection between any two carbon atoms marked with the * connected by a moiety -CL-, which degradable bonds may be the same or different. In certain embodiments there are four degradable bonds selected from the group consisting of ester, carbonate, sulfate, phosphate bonds, carbamate and amide bonds in the direct connection between any two carbon atoms marked with the * connected by a moiety -CL-, which degradable bonds may be the same or different. In certain embodiments there are five degradable bonds selected from the group consisting of ester, carbonate, sulfate, phosphate bonds, carbamate and amide bonds in the direct connection between any two carbon atoms marked with the * connected by a moiety -CL-, which degradable bonds may be the same or different. In certain embodiments there are six degradable bonds selected from the group consisting of ester, carbonate, sulfate, phosphate bonds, carbamate and amide bonds in the direct connection between any two carbon atoms marked with the * connected by a moiety -CL-, which degradable bonds may be the same or different. It is understood that if more than two units Z³ are connected by -CL- there are more than two carbons marked with * that are connected and thus there is more than one shortest connection with at least one degradable bond present. Each shortest connection may have the same or different number of degradable bonds.

In certain embodiments the at least one degradable bond, such as one, two, three, four, five, six degradable bonds, are located within -CL-.

In certain embodiments the at least one degradable bond present in the direct connection between any two carbon atoms marked with * connected by a moiety -CL- is one ester bond. In other embodiments the at least one degradable bond are two ester bonds. In other embodiments the at least one degradable bond are three ester bonds. In other embodiments the at least one degradable bond are four ester bonds. In other embodiments the at least one degradable bond are five ester bonds. In other embodiments the at least one degradable bond are six ester bonds.

In certain embodiments the at least one degradable bond present in the direct connection between any two carbon atoms marked with * connected by a moiety -CL- is one carbonate bond. In other embodiments the at least one degradable bond are two carbonate bonds. In other embodiments the at least one degradable bond are three carbonate bonds. In other embodiments the at least one degradable bond are four carbonate bonds. In other embodiments the at least one degradable bond are five carbonate bonds. In other embodiments the at least one degradable bond are six carbonate bonds.

In certain embodiments the at least one degradable bond present in the direct connection between any two carbon atoms marked with * connected by a moiety -CL- is one phosphate bond. In other embodiments the at least one degradable bond are two phosphate bonds. In other embodiments the at least one degradable bond are three phosphate bonds. In other embodiments the at least one degradable bond are four phosphate bonds. In other embodiments the at least one degradable bond are five phosphate bonds. In other embodiments the at least one degradable bond are six phosphate bonds.

In certain embodiments the at least one degradable bond present in the direct connection between any two carbon atoms marked with * connected by a moiety -CL- is one sulfate bond. In other embodiments the at least one degradable bond are two sulfate bonds. In other embodiments the at least one degradable bond are three sulfate bonds. In other embodiments the at least one degradable bond are four sulfate bonds. In other embodiments the at least one degradable bond are five sulfate bonds. In other embodiments the at least one degradable bond are six sulfate bonds.

In certain embodiments the at least one degradable bond present in the direct connection between any two carbon atoms marked with * connected by a moiety -CL- is one carbamate bond. In other embodiments the at least one degradable bond are two carbamate bonds. In other embodiments the at least one degradable bond are three carbamate bonds. In other embodiments the at least one degradable bond are four carbamate bonds. In other embodiments the at least one degradable bond are five carbamate bonds. In other embodiments the at least one degradable bond are six carbamate bonds.

In certain embodiments the at least one degradable bond present in the direct connection between any two carbon atoms marked with * connected by a moiety -CL- is one amide bond. In other embodiments the at least one degradable bond are two amide bonds. In other embodiments the at least one degradable bond are three amide bonds. In other embodiments the at least one degradable bond are four amide bonds. In other embodiments the at least one degradable bond are five amide bonds. In other embodiments the at least one degradable bond are six amide bonds.

It was found that a high degree of derivatization of the disaccharide units of hyaluronic acid, meaning that the number of units Z¹ is less than 80% of all units present in the conjugate, interferes with degradation of the hydrogel by certain hyaluronidases. This has the effect that less degradation by hyaluronidases occurs and that chemical cleavage of the degradable bonds becomes more relevant. This renders degradation of the conjugate more predictable. The reason for this is that the level of enzymes, such as hyaluronidases, exhibits inter-patient variability and may vary between different administration sites, whereas chemical cleavage predominantly depends on temperature and pH which are more stable parameters and thus chemical cleavage tends to be more predictable.

In some embodiments -CL- is C₁₋₅₀ alkyl, which is optionally interrupted by one or more atoms or groups selected from the group consisting of -T-, —C(O)O—, —O—, —C(O)—, —C(O)N(R^{c1})—, —S(O)₂—, —S(O)—, —S—, —N(R^{c1})—, —OC(OR^{c1})(Rc^{1a})_ and —OC(O)N(R^{c1})—;
wherein -T- is selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; and
—R^{c1} and —R^{c1a} are selected from the group consisting of —H and C₁₋₆ alkyl.

In certain embodiments -CL- is a moiety of formula (A)

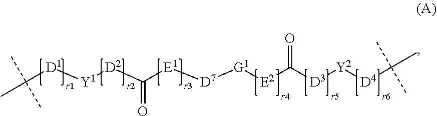

wherein
—Y¹— is of formula

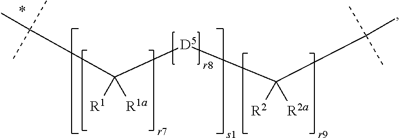

wherein the dashed line marked with the asterisk indicates attachment to -D¹- and the unmarked dashed line indicates attachment to -D²-;
—Y²— is of formula

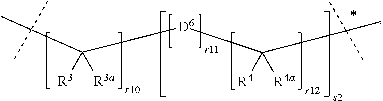

wherein the dashed line marked with the asterisk indicates attachment to -D⁴- and the unmarked dashed line indicates attachment to -D³-;
-E¹- is of formula

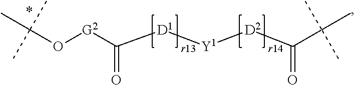

wherein the dashed line marked with the asterisk indicates attachment to —(C═O)— and the unmarked dashed line indicates attachment to —O—;
-E²- is of formula

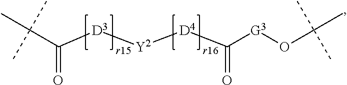

wherein the dashed line marked with the asterisk indicates attachment to -G¹- and the unmarked dashed line indicates attachment to —(C═O)—;
-G¹- is of formula

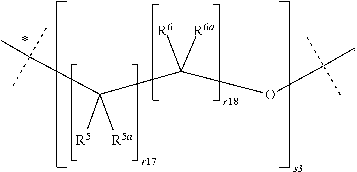

wherein the dashed line marked with the asterisk indicates attachment to —O— and the unmarked dashed line indicates attachment to -E²-;
-G²- is of formula

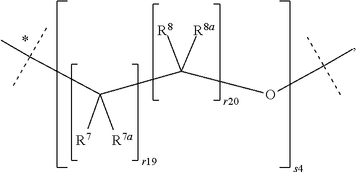

wherein the dashed line marked with the asterisk indicates attachment to —O— and the unmarked dashed line indicates attachment to —(C═O)—;
-G³- is of formula

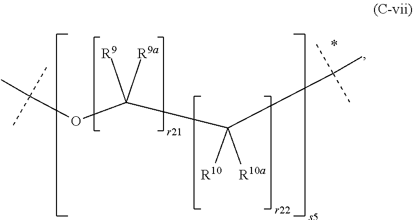

wherein the dashed line marked with the asterisk indicates attachment to —O— and the unmarked dashed line indicates attachment to —(C═O)—;
-D¹-, -D²-, -D³-, -D⁴-, -D⁵-, -D⁶- and -D⁷- are identical or different and each is independently of the others selected from the group comprising —O—, —NR¹¹—, —N⁺R¹²R^{12a}, —S—, —(S═O)—, —(S(O)₂), —C(O)—, —P(O)R¹³ and —CR¹⁴R^{14a}—;
—R¹, —R^{1a}, —R², —R^{2a}, —R³, —R^{3a}, —R⁴, —R^{4a}, —R⁵, —R^{5a}, —R⁶, —R^{6a}, —R⁷, —R^{7a}, —R⁸, —R^{8a}, —R⁹, —R^{9a}, —R¹⁰, —R^{10a}, —R¹¹, —R¹², —R^{12a}, —R¹³, —R¹⁴ and —R^{14a} are identical or different and each are identical or different and each is independently of the others selected from the group comprising —H and C₁₋₆ alkyl;
optionally, one or more of the pairs —R¹/—R^{1a}, —R²/—R^{2a}, —R³/—R^{3a}, —R⁴/—R^{4a}, —R¹/—R², —R³/—R⁴, —R^{1a}/—R^{2a}, —R^{3a}/—R^{4a}, —R¹²/—R^{12a}, and —R¹⁴/—R^{14a} form a chemical bond or are joined together with the atom to which they are attached to form a C₃₋₈ cycloalkyl or to form a ring A or are joined together with the atom to which they are attached to form a 4- to 7-membered heterocyclyl or 8- to 11-membered heterobicyclyl or adamantyl;
A is selected from the group consisting of phenyl, naphthyl, indenyl, indanyl and tetralinyl;
r1, r2, r5, r6, r13, r14, r15 and r16 are independently 0 or 1;
r3, r4, r7, r8, r9, r10, r11, r12 are independently 0, 1, 2, 3, or 4;
r17, r18, r19, r20, r21 and r22 are independently 1, 2, 3, 4, 5, 6, 7, 8, 9 or 10; and
s1, s2, s4, s5 are independently 1, 2, 3, 4, 5 or 6.
s3 ranges from 1 to 200, preferably from 1 to 100 and more preferably from 1 to 50

In certain embodiments r1 of formula (A) is 0. In certain embodiments r1 of formula (A) is 1. In certain embodiments r2 of formula (A) is 0. In certain embodiments r2 of formula (A) is 1. In certain embodiments r5 of formula (A) is 0. In certain embodiments r5 of formula (A) is 1. In certain embodiments r6 of formula (A) is 0. In certain embodiments r6 of formula (A) is 1. In certain embodiments r13 of formula (A) is 0. In certain embodiments r13 of formula (A) is 1. In certain embodiments r14 of formula (A) is 0. In certain embodiments r14 of formula (A) is 1. In certain embodiments r15 of formula (A) is 0. In certain embodiments r15 of formula (A) is 1. In certain embodiments r16 of formula (A) is 0. In certain embodiments r16 of formula (A) is 1.

In certain embodiments r3 of formula (A) is 0. In certain embodiments r3 of formula (A) is 1. In certain embodiments r4 of formula (A) is 0. In certain embodiments r4 of formula (A) is 1. In certain embodiments r3 of formula (A) and r4 of formula (A) are both 0.

In certain embodiments r7 of formula (A) is 0. In certain embodiments r7 of formula (A) is 1. In certain embodiments r7 of formula (A) is 2. In certain embodiments r8 of formula (A) is 0. In certain embodiments r8 of formula (A) is 1. In certain embodiments r8 of formula (A) of formula (A) is 2. In certain embodiments r9 of formula (A) is 0. In certain embodiments r9 of formula (A) is 1. In certain embodiments r9 of formula (A) is 2. In certain embodiments r10 of formula (A) is 0. In certain embodiments r10 of formula (A) is 1. In certain embodiments r10 of formula (A) is 2. In certain embodiments r11 of formula (A) is 0. In certain embodiments r11 of formula (A) is 1. In certain embodiments r11 of formula (A) is 2. In certain embodiments r12 of formula (A) is 0. In certain embodiments r12 of formula (A) is 1. In certain embodiments r12 of formula (A) is 2.

In certain embodiments r17 of formula (A) is 1. In certain embodiments r18 of formula (A) is 1. In certain embodiments r19 of formula (A) is 1. In certain embodiments r20 of formula (A) is 1. In certain embodiments r21 of formula (A) is 1.

In certain embodiments s1 of formula (A) is 1. In certain embodiments s1 of formula (A) is 2. In certain embodiments s2 of formula (A) is 1. In certain embodiments s2 of formula (A) is 2. In certain embodiments s4 of formula (A) is 1. In certain embodiments s4 of formula (A) is 2.

In certain embodiments s3 of formula (A) ranges from 1 to 100. In certain embodiments s3 of formula (A) ranges from 1 to 75. In certain embodiments s3 of formula (A) ranges from 2 to 50. In certain embodiments s3 of formula (A) ranges from 2 to 40. In certain embodiments s3 of formula (A) ranges from 3 to 30. In certain embodiments s3 of formula (A) is about 3.

In certain embodiments —R¹ of formula (A) is —H. In certain embodiments —R¹ of formula (A) is methyl. In certain embodiments —R¹ of formula (A) is ethyl. In certain embodiments —R^{1a} of formula (A) is —H. In certain embodiments —R^{1a} of formula (A) is methyl. In certain embodiments —R^{1a} of formula (A) is ethyl. In certain embodiments —R² of formula (A) is —H. In certain embodiments —R² of formula (A) is methyl. In certain embodiments —R² of formula (A) is ethyl. In certain embodiments —R^{2a} of formula (A) is —H. In certain embodiments —R^{2a} of formula (A) is methyl. In certain embodiments —R^{2a} of formula (A) is ethyl. In certain embodiments —R³ of formula (A) is —H. In certain embodiments —R³ of formula (A) is methyl. In certain embodiments —R³ of formula (A) is ethyl. In certain embodiments —R^{3a} of formula (A) is —H. In certain embodiments —R^{3a} of formula (A) is methyl. In certain embodiments —R^{3a} of formula (A) is ethyl. In certain embodiments —R⁴ of formula (A) is —H. In certain embodiments —R⁴ of formula (A) is methyl. In certain embodiments —R⁴ of formula (A) is methyl. In certain embodiments —R^{4a} of formula (A) is —H. In certain embodiments —R^{4a} of formula (A) is methyl. In certain embodiments —R^{4a} of formula (A) is ethyl. In certain embodiments —R⁵ of formula (A) is —H. In certain embodiments —R⁵ of formula (A) is methyl. In certain embodiments —R⁵ of formula (A) is ethyl. In certain embodiments —R^{5a} of formula (A) is —H. In certain embodiments —R^{5a} of formula (A) is methyl. In certain embodiments —R^{5a} of formula (A) is ethyl. In certain embodiments —R⁶ of formula (A) is —H. In certain embodiments —R⁶ of formula (A) is methyl. In certain embodiments —R⁶ of formula (A) is ethyl. In certain embodiments —R^{6a} of formula (A) is —H. In certain embodiments —R^{6a} of formula (A) is methyl. In certain embodiments —R^{6a} of formula (A) is ethyl. In certain embodiments —R⁷ of formula (A) is —H. In certain embodiments —R⁷ of formula (A) is methyl. In certain embodiments —R⁷ of formula (A) is ethyl. In certain embodiments —R⁸ of formula (A) is —H. In certain embodiments —R⁸ of formula (A) is methyl. In certain embodiments —R⁸ of formula (A) is ethyl. In certain embodiments —R^{8a} of formula (A) is —H. In certain embodiments —R^{8a} of formula (A) is methyl. In certain embodiments —R^{8a} of formula (A) is ethyl. In certain embodiments —R⁹ of formula (A) is —H. In certain embodiments —R⁹ of formula (A) is methyl. In certain embodiments —R⁹ of formula (A) is ethyl. In certain embodiments —R^{9a} of formula (A) is —H. In certain embodiments —R^{9a} of formula (A) is methyl. In certain embodiments —R^{9a} of formula (A) is ethyl. In certain embodiments —R¹⁰ of formula (A) is —H. In certain embodiments —R¹⁰ of formula (A) is methyl. In certain embodiments —R¹⁰ of formula (A) is ethyl. In certain embodiments —R^{10a} of formula (A) is —H. In certain embodiments —R^{10a} of formula (A) is methyl. In certain embodiments —R^{10a} of formula (A) is ethyl. In certain embodiments —R¹¹ of formula (A) is —H. In certain embodiments —R¹¹ of formula (A) is methyl. In certain embodiments —R¹¹ of formula (A) is ethyl. In certain embodiments —R¹² of formula (A) is —H. In certain embodiments —R¹² of formula (A) is methyl. In certain embodiments —R¹² of formula (A) is ethyl. In certain embodiments —R^{12a} of formula (A) is —H. In certain embodiments —R^{12a} of formula (A) is methyl. In certain embodiments —R^{12a} of formula (A) is ethyl. In certain embodiments —R¹³ of formula (A) is —H. In certain embodiments —R¹³ of formula (A) is methyl. In certain embodiments —R¹³ of formula (A) is ethyl. In certain embodiments —R¹⁴ of formula (A) is —H. In certain embodiments —R¹⁴ of formula (A) is methyl. In certain embodiments —R¹⁴ of formula (A) is ethyl. In certain embodiments —R^{14a} of formula (A) is —H. In certain embodiments —R^{14a} of formula (A) is methyl. In certain embodiments —R^{14a} of formula (A) is ethyl.

In certain embodiments -D¹- of formula (A) is —O—. In certain embodiments -D¹- of formula (A) is —NR¹¹—. In certain embodiments -D¹- of formula (A) is —N⁺R¹²R^{12a}—. In certain embodiments -D¹- of formula (A) is —S—. In certain embodiments -D¹- of formula (A) is —(S═O). In certain embodiments -D¹- of formula (A) is —(S(O)₂)—. In certain embodiments -D¹- of formula (A) is —C(O)—. In certain embodiments -D¹- of formula (A) is —P(O)R¹³—. In certain embodiments -D¹- of formula (A) is —P(O)(OR¹³)—. In certain embodiments -D¹- of formula (A) is —CR¹⁴R^{14a}—.

In certain embodiments -D²- of formula (A) is —O—. In certain embodiments -D²- of formula (A) is —NR¹¹—. In certain embodiments -D²- of formula (A) is —N⁺R¹²R^{12a}—. In certain embodiments -D²- of formula (A) is —S—. In certain embodiments -D²- of formula (A) is —(S═O). In certain embodiments -D²- of formula (A) is —(S(O)₂)—. In certain embodiments -D²- of formula (A) is —C(O)—. In certain embodiments -D²- of formula (A) is —P(O)R¹³—. In certain embodiments -D²- of formula (A) is —P(O)(OR¹³)—. In certain embodiments -D²- of formula (A) is —CR¹⁴R^{14a}—.

In certain embodiments -D³- of formula (A) is —O—. In certain embodiments -D³- of formula (A) is —NR¹¹—. In certain embodiments -D³- of formula (A) is —N⁺R¹²R^{12a}—. In certain embodiments -D³- of formula (A) is —S—. In certain embodiments -D³- of formula (A) is —(S═O). In certain embodiments -D³- of formula (A) is —(S(O)₂)—. In certain embodiments -D³- of formula (A) is —C(O)—. In certain embodiments -D³- of formula (A) is —P(O)R¹³—. In certain embodiments -D³- of formula (A) is —P(O)(OR¹³)—. In certain embodiments -D³- of formula (A) is —CR¹⁴R^{14a}—.

In certain embodiments -D⁴- of formula (A) is —O—. In certain embodiments -D⁴- of formula (A) is —NR¹¹—. In certain embodiments -D⁴- of formula (A) is —N⁺R¹²R^{12a}—. In certain embodiments -D⁴- of formula (A) is —S—. In certain embodiments -D⁴- of formula (A) is —(S═O). In certain embodiments -D⁴- of formula (A) is —(S(O)₂)—. In certain embodiments -D⁴- of formula (A) is —C(O)—. In certain embodiments -D⁴- of formula (A) is —P(O)R¹³—. In certain embodiments -D⁴- of formula (A) is —P(O)(OR¹³)—. In certain embodiments -D⁴- of formula (A) is —CR¹⁴R^{14a}—.

In certain embodiments -D⁵- of formula (A) is —O—. In certain embodiments -D⁵- of formula (A) is —NR¹¹—. In certain embodiments -D⁵- of formula (A) is —N⁺R¹²R^{12a}—. In certain embodiments -D⁵- of formula (A) is —S—. In certain embodiments -D⁵- of formula (A) is —(S═O)—. In certain embodiments -D⁵- of formula (A) is —(S(O)₂)—. In certain embodiments -D⁵- of formula (A) is —C(O)—. In certain embodiments -D⁵- of formula (A) is —P(O)R¹³—. In certain embodiments -D⁵- of formula (A) is —P(O)(OR¹³)—. In certain embodiments -D⁵- of formula (A) is —CR¹⁴R^{14a}—.

In certain embodiments -D⁶- of formula (A) is —O—. In certain embodiments -D⁶- of formula (A) is —NR¹¹—. In certain embodiments -D⁶- of formula (A) is —N⁺R¹²R^{12a}—. In certain embodiments -D⁶- of formula (A) is —S—. In certain embodiments -D⁶- of formula (A) is —(S═O). In certain embodiments -D⁶- of formula (A) is —(S(O)₂)—. In certain embodiments -D⁶- of formula (A) is —C(O)—. In certain embodiments -D⁶- of formula (A) is —P(O)R¹³—. In certain embodiments -D⁶- of formula (A) is —P(O)(OR¹³)—. In certain embodiments -D⁶- of formula (A) is —CR¹⁴R^{14a}—.

In certain embodiments -D⁷- of formula (A) is —O—. In certain embodiments -D⁷- of formula (A) is —NR¹¹—. In certain embodiments -D⁷- of formula (A) is —N⁺R¹²R^{12a}—. In certain embodiments -D⁷- of formula (A) is —S—. In certain embodiments -D⁷- of formula (A) is —(S═O). In certain embodiments -D⁷- of formula (A) is —(S(O)₂)—. In certain embodiments -D⁷- of formula (A) is —C(O)—. In certain embodiments -D⁷- of formula (A) is —P(O)R¹³—. In certain embodiments -D⁷- of formula (A) is —P(O)(OR¹³)—. In certain embodiments -D⁷- of formula (A) is —CR¹⁴R^{14a}—.

In certain embodiments -CL- is of formula (B)

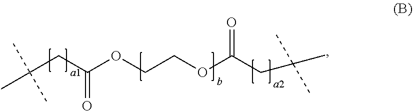

wherein
a1 and a2 are independently selected from the group consisting of a1 and a2 are independently selected from the group consisting of 1, 2, 3, 4, 5, 6, 7, 8, 9, 10, 11, 12, 13 and 14; and
b is an integer ranging from 1 to 50.

In certain embodiments a1 and a2 of formula (B) are different. In certain embodiments a1 and a2 of formula (B) are the same.

In certain embodiments a1 of formula (B) is 1. In certain embodiments a1 of formula (B) is 2. In certain embodiments a1 of formula (B) is 3. In certain embodiments a1 of formula (B) is 4. In certain embodiments a1 of formula (B) is 5. In certain embodiments a1 of formula (B) is 6. In certain embodiments a1 of formula (B) is 7. In certain embodiments a1 of formula (B) is 8. In certain embodiments a1 of formula (B) is 9. In certain embodiments a1 of formula (B) is 10.

In certain embodiments a2 of formula (B) is 1. In certain embodiments a2 of formula (B) is 2. In certain embodiments a2 of formula (B) is 3. In certain embodiments a2 of formula (B) is 4. In certain embodiments a2 of formula (B) is 5. In certain embodiments a2 of formula (B) is 6. In certain embodiments a2 of formula (B) is 7. In certain embodiments a2 of formula (B) is 8. In certain embodiments a2 of formula (B) is 9. In certain embodiments a2 of formula (B) is 10. In certain embodiments b of formula (B) ranges from 1 to 500. In certain embodiments b of formula (B) ranges from 2 to 250. In certain embodiments b of formula (B) ranges from 3 to 100. In certain embodiments b of formula (B) ranges from 3 to 50. In certain embodiments b of formula (B) ranges from 3 to 25. In certain embodiments b of formula (B) is 3. In certain embodiments b of formula (B) is 25.

In certain embodiments -CL- is of formula (B-i)

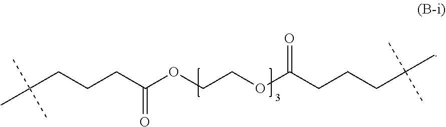

In certain embodiments -CL- is of formula (C)

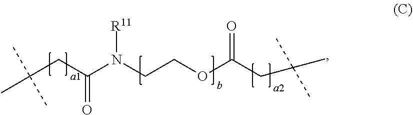

wherein
a1 and a2 are independently selected from the group consisting of 1, 2, 3, 4, 5, 6, 7, 8, 9, 10, 11, 12, 13 and 14;
b is an integer ranging from 1 to 50; and
—R¹¹ is selected from the group comprising —H and C₁₋₆ alkyl.

In certain embodiments a1 and a2 of formula (C) are different. In certain embodiments a1 and a2 of formula (B) are the same.

In certain embodiments a1 of formula (C) is 1. In certain embodiments a1 of formula (C) is 2. In certain embodiments a1 of formula (C) is 3. In certain embodiments a1 of formula (C) is 4. In certain embodiments a1 of formula (C) is 5. In certain embodiments a1 of formula (C) is 6. In certain embodiments a1 of formula (C) is 7. In certain embodiments a1 of formula (C) is 8. In certain embodiments a1 of formula (C) is 9. In certain embodiments a1 of formula (C) is 10. In certain embodiments a2 of formula (C) is 1. In certain embodiments a2 of formula (C) is 2. In certain embodiments a2 of formula (C) is 3. In certain embodiments a2 of formula (C) is 4. In certain embodiments a2 of formula (C) is 5. In certain embodiments a2 of formula (C) is 6. In certain embodiments a2 of formula (C) is 7. In certain embodiments a2 of formula (C) is 8. In certain embodiments a2 of formula (C) is 9. In certain embodiments a2 of formula (C) is 10.

In certain embodiments b of formula (C) ranges from 1 to 500. In certain embodiments b of formula (C) ranges from 2 to 250. In certain embodiments b of formula (C) ranges from 3 to 100. In certain embodiments b of formula (C) ranges from 3 to 50. In certain embodiments b of formula (C) ranges from 3 to 25. In certain embodiments b of formula (C) is 3. In certain embodiments b of formula (C) is 25.

In certain embodiments —R¹¹ of formula (C) is —H. In certain embodiments —R¹¹ of formula (C) is methyl. In certain embodiments —R¹¹ of formula (C) is ethyl. In certain embodiments —R¹¹ of formula (C) is n-propyl. In certain embodiments —R¹¹ of formula (C) is isopropyl. In certain embodiments —R¹¹ of formula (C) is n-butyl. In certain embodiments —R¹¹ of formula (C) is isobutyl. In certain embodiments —R¹¹ of formula (C) is sec-butyl. In certain embodiments —R¹¹ of formula (C) is tert-butyl. In certain embodiments —R¹¹ of formula (C) is n-pentyl. In certain embodiments —R¹¹ of formula (C) is 2-methylbutyl. In certain embodiments —R¹¹ of formula (C) is 2,2-dimethylpropyl. In certain embodiments —R¹¹ of formula (C) is n-hexyl. In certain embodiments —R¹¹ of formula (C) is 2-methylpentyl. In certain embodiments —R¹¹ of formula (C) is 3-methylpentyl. In certain embodiments —R¹¹ of formula (C) is 2,2-dimethylbutyl. In certain embodiments —R¹¹ of formula (C) is 2,3-dimethylbutyl. In certain embodiments —R¹¹ of formula (C) is 3,3-dimethylpropyl.

In certain embodiments -CL- is of formula (C-i)

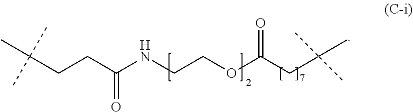

In a second embodiment the moiety -CL- is selected from the group consisting of

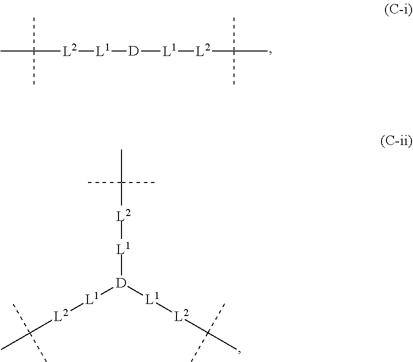

wherein
each dashed line indicates attachment to a unit Z³; and
-L²- and -D are used as defined for Z².

It is understood that in formula (C-i) two functional groups of the drug are conjugated to one moiety -L¹- each and that in formula (C-ii) three functional groups of the drug are conjugated to one moiety -L¹- each. The moiety -CL- of formula (C-i) connects two moieties Z³ and the moiety -CL- of formula (C-ii) connects three moieties Z³, which may be on the same or different hyaluronic acid strand. In this embodiment -CL- comprises at least two degradable bonds, if -CL- is of formula (C-i) or at least three degradable bonds, if -CL- is of formula (C-ii), namely the degradable bonds that connect D with a moiety -L¹-. A conjugate may only comprise moieties -CL- of formula (C-i), may only comprise moieties -CL- of formula (C-ii) or may comprise moieties -CL- of formula (C-i) and formula (C-ii).

Accordingly, a conjugate of this second embodiment comprises crosslinked hyaluronic acid strands to which a plurality of drug moieties are covalently and reversibly conjugated, wherein the conjugate comprises a plurality of connected units selected from the group consisting of

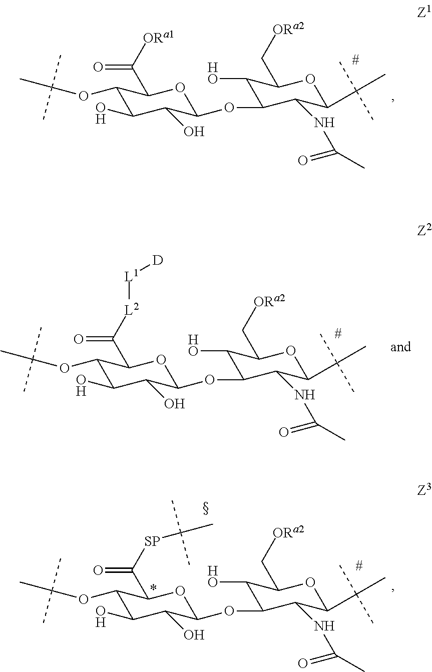

wherein
an unmarked dashed line indicates a point of attachment to an adjacent unit at a dashed line marked with # or to a hydrogen;
a dashed line marked with # indicates a point of attachment to an adjacent unit at an unmarked dashed line or to a hydroxyl;
a dashed line marked with § indicates a point of connection between at least two units Z³ via a moiety -CL-;
each -CL- comprises at least one degradable bond between the two carbon atoms marked with the * connected by a moiety -CL- and each -CL- is independently selected from the group consisting of formula (C-i) and (C-ii)

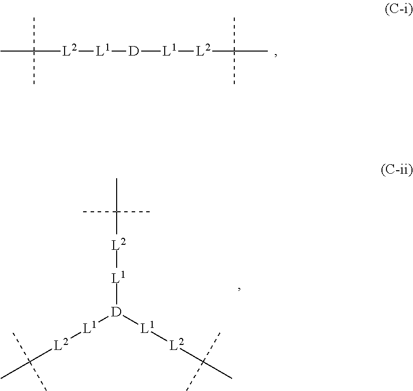

wherein
dashed lines indicate attachment to a unit Z³;
-D, -L¹-, -L²-, -SP-, —R^{a1} and —R^{a2} are used as defined for Z¹, Z² and Z³;
wherein
all units Z¹ present in the conjugate may be the same or different;
all units Z² present in the conjugate may be the same or different;
all units Z³ present in the conjugate may be the same or different;
the number of Z¹ units ranges from 1% to 98% of the total number of units present in the conjugate;
the number of Z² units ranges from 0% to 98% of the total number of units present in the conjugate;
the number of Z³ units ranges from 1% to 97% of the total number of units present in the conjugate, provided that at least one unit Z³ is present per strand which is connected to at least one unit Z³ on a different hyaluronic acid strand.

It is understood that such hydrogel according to the second embodiment also comprises partly reacted or unreacted units and that the presence of such moieties cannot be avoided. In certain embodiments the sum of such partly reacted or unreacted units is no more than 25% of the total number of units present in the conjugate, such as no more than 20%, such as no more than 15% or such as no more than 10%.

In a conjugate according to this second embodiment the number of units Z² ranges from 0 to 70% of all units present in the conjugate, such as from 2 to 15%, from 2 to 10%, from 16 to 39, from 40 to 65%, or from 50 to 60% of all units present in the conjugate.

In a conjugate according to this second embodiment the number of units Z³ ranges from 1 to 30% of all units present in the conjugate, such as from 2 to 5%, from 5 to 20%, from 10 to 18%, or from 14 to 18% of all units present in the conjugate.

In a conjugate according to this second embodiment the number of units Z¹ ranges from 10 to 97% of all units present in the conjugate, such as from 20 to 40%, such as from 25 to 35%, such as from 41 to 95%, such as from 45 to 90%, such as from 50 to 70% of all units present in the conjugate.

More specific embodiments for -D, -L¹-, -L²-, -SP-, —R^{a1} and —R^{a2} of the second embodiment are as described elsewhere herein.

In a third embodiment the moiety -CL- is a moiety

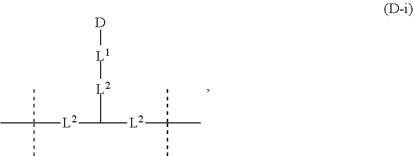

wherein
each dashed line indicates attachment to a unit Z³.

It is understood that a moiety -CL- of formula (D-i) comprises at least one branching point, which branching point may be selected from the group consisting of

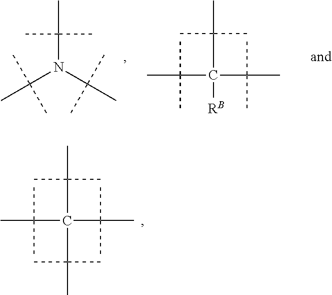

wherein
dashed lines indicate attachment to an arm; and
—R¹³ is selected from the group consisting of —H, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R^{B1}, which are the same or different, and wherein C₁₋₆ alkyl, C₂₋₆ alkenyl and C₂₋₆ alkynyl are optionally interrupted with —C(O)O—, —O—, —C(O)—, —C(O)N(R^{B2})—, —S(O)₂N(R^{B2})—, —S(O)N(R^{B2})—, —S(O)₂—, —S(O)—, —N(R^{B2})S(O)₂N(R^{B2a})—, —S—, —N(R^{B2})—, —OC(OR^{B2})(R^{B2a}), —N(R^{B2})C(O)N(RB^{2a})—, and —OC(O)N(R^{B2})—; wherein —R^{B1}, —R^{B2} and —R^{B2a} are selected from —H, C₁₋₆ alkyl, C₂₋₆ alkenyl and C₂₋₆ alkynyl.

In certain embodiments —R^{B} is selected from the group consisting of —H, methyl and ethyl.

Accordingly, a conjugate of the third embodiment comprises crosslinked hyaluronic acid strands to which a plurality of drug moieties are covalently and reversibly conjugated, wherein the conjugate comprises a plurality of connected units selected from the group consisting of

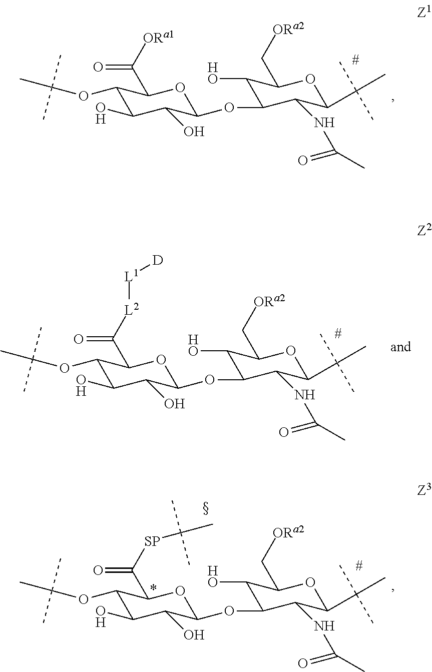

wherein
an unmarked dashed line indicates a point of attachment to an adjacent unit at a dashed line marked with # or to a hydrogen;
a dashed line marked with # indicates a point of attachment to an adjacent unit at an unmarked dashed line or to a hydroxyl;
a dashed line marked with § indicates a point of connection between two units Z³ via a moiety -CL-;
each -CL- comprises at least one degradable bond between the two carbon atoms marked with the * connected by a moiety -CL- and each -CL- is independently of formula (D-i)

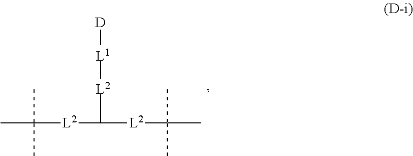

wherein
dashed lines indicate attachment to a unit Z³;
-D, -L¹-, -L²-, -SP-, —R^{a1} and —R^{a2} are used as defined for Z¹, Z² and Z³;
wherein
all units Z¹ present in the conjugate may be the same or different;
all units Z² present in the conjugate may be the same or different;
all units Z³ present in the conjugate may be the same or different;
the number of units Z¹ ranges from 1% to 99% of the total number of units present in the conjugate;
the number of units Z² ranges from 0% to 98% of the total number of units present in the conjugate; and
the number of units Z³ ranges from 1% to 97% of the total number of units present in the conjugate, provided that at least one unit Z³ is present per strand.

It is understood that such hydrogel according to the third embodiment also comprises partly reacted or unreacted units and that the presence of such moieties cannot be avoided. In certain embodiments the sum of such partly reacted or unreacted units is no more than 25% of the total number of units present in the conjugate, such as no more than 10%, such as no more than 15% or such as no more than 10%.

In a conjugate according to this third embodiment the number of units Z² ranges from 0 to 70% of all units present in the conjugate, such as from 2 to 15%, from 2 to 10%, from 16 to 39, from 40 to 65%, or from 50 to 60% of all units present in the conjugate.

In a conjugate according to this third embodiment the number of units Z³ ranges from 1 to 30% of all units present in the conjugate, such as from 2 to 5%, from 5 to 20%, from 10 to 18%, or from 14 to 18% of all units present in the conjugate.

In a conjugate according to this third embodiment the number of units Z¹ ranges from 10 to 97% of all units present in the conjugate, such as from 20 to 40%, such as from 25 to 35%, such as from 41 to 95%, such as from 45 to 90%, such as from 50 to 70% of all units present in the conjugate.

In this third embodiment -CL- comprises a moiety -L²-L¹-D, so the presence of units Z² is optional in this embodiment. In certain embodiment no units Z² are present in the third embodiment. In certain embodiments the conjugate according to the third embodiment also comprises units Z². The presence of units Z² may have the effect that in case of a high drug loading is desired, which in this embodiment also means a high degree of crosslinking, an undesired high degree of crosslinking can be avoided by the presence of units Z².

More specific embodiments for -D, -L²-, -SP-, —R^{a1} and —R^{a2} of the second embodiment are as described elsewhere herein.

-SP- is absent or a spacer moiety. In certain embodiments -SP- does not comprise a reversible linkage, i.e. all linkages in -SP- are stable linkages.

In certain embodiments -SP- is absent.

In certain embodiments -SP- is a spacer moiety.

In certain embodiments -SP- does not comprise a degradable bond, i.e. all bonds of -SP- are stable bonds. In certain embodiments at least one of the at least one degradable bond in the direct connection between two carbon atoms marked with the * connected by a moiety -CL- is provided by -SP-.

In certain embodiments -SP- is a spacer moiety selected from the group consisting of -T-, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;

—R^{y1} and —R^{y1a} are independently of each other selected from the group consisting of —H, -T, C₁₋₅₀ alkyl, C₂₋₅₀ alkenyl, and C₂₋₅₀ alkynyl; wherein -T, C₁₋₅₀ alkyl, C₂₋₅₀ alkenyl, and C₂₋₅₀ alkynyl are optionally substituted with one or more —R^{y2}, which are the same or different, and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;

each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;

each —R^{y2} is independently selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and

each —R^{y3}, —R^{y3a}, —R^{y4}, —R^{y4a}, —R^{y5}, —R^{y5a} and —R^{y5b} is independently selected from the group consisting of —H, and C₁₋₆ alkyl, wherein C₁₋₆ alkyl is optionally substituted with one or more halogen, which are the same or different.

In certain embodiments -SP- is a spacer moiety selected from the group consisting of -T-, C₁₋₅₀ alkyl, C₂₋₅₀ alkenyl, and C₂₋₅₀ alkynyl; wherein -T-, C₁₋₂₀ alkyl, C₂₋₂₀ alkenyl, and C₂₋₂₀ alkynyl are optionally substituted with one or more —R^{y2}, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;

—R^{y1} and —R^{y1a} are independently of each other selected from the group consisting of —H, -T, C₁₋₁₀ alkyl, C₂₋₁₀ alkenyl, and C₂₋₁₀ alkynyl; wherein -T, C₁₋₁₀ alkyl, C₂₋₁₀ alkenyl, and C₂₋₁₀ alkynyl are optionally substituted with one or more —R^{y2}, which are the same or different, and wherein C₁₋₁₀ alkyl, C₂₋₁₀ alkenyl, and C₂₋₁₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;

each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;

—R^{y2} is selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and

each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C₁₋₆ alkyl is optionally substituted with one or more halogen, which are the same or different.

In certain embodiments -SP- is a spacer moiety selected from the group consisting of -T-, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;

—R^{y1} and —R^{y1a} are independently selected from the group consisting of —H, -T, C₁₋₁₀ alkyl, C₂₋₁₀ alkenyl, and C₂₋₁₀ alkynyl;

each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl;

each —R^{y2} is independently selected from the group consisting of halogen and C₁₋₆ alkyl; and

each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C₁₋₆ alkyl is optionally substituted with one or more halogen, which are the same or different.

In certain embodiments -SP- is a C₁₋₂₀ alkyl chain, which is optionally interrupted by one or more groups independently selected from —O—, -T-, —N(R^{y3})— and —C(O)N(R^{y1})—; and which C₁₋₂₀ alkyl chain is optionally substituted with one or more groups independently selected from —OH, -T, —N(R^{y3})— and —C(O)N(R^{y6}R^{y6a}); wherein —R^{y1}, —R^{y6}, —R^{y6a} are independently selected from the group consisting of H and C₁₋₄ alkyl, wherein T is selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl, provided that -SP- is attached to —X^{0E}— and —X^{0F}— via a carbon atom of -SP-.

In certain embodiments -SP- has a molecular weight ranging from 14 g/mol to 750 g/mol.

In certain embodiments -SP- has a chain length ranging from 1 to 20 atoms.

In certain embodiments all moieties -SP- of a conjugate are identical.

In certain embodiments -SP- is a C₁₋₁₀ alkyl. In certain embodiments -SP- is a C₁ alkyl. In certain embodiments -SP- is a C₂ alkyl. In certain embodiments -SP- is a C₃ alkyl. In certain embodiments -SP- is a C₄ alkyl. In certain embodiments -SP- is a C₅ alkyl. In certain embodiments -SP- is a C₆ alkyl. In certain embodiments -SP- is a C₇ alkyl. In certain embodiments -SP- is a C₈ alkyl. In certain embodiments -SP- is a C₉ alkyl. In certain embodiments -SP- is a C₁₀ alkyl.

In certain embodiments Z is a hydrogel as disclosed in WO2013/036847 A1. In particular, in certain embodiments Z is a hydrogel produced by a method comprising the step of reacting at least a first reactive polymer with a cleavable crosslinker compound, wherein said cleavable crosslinker compound comprises a first functional group —Y¹ that reacts with the first reactive polymer and further comprises a moiety that is cleaved by elimination under physiological conditions wherein said moiety comprises a second functional group —Y² that reacts with a second reactive polymer. In certain embodiments the cleavable crosslinker compound is of formula (PL-1)

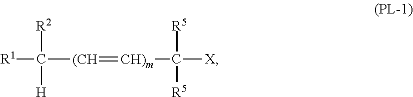

wherein
m is 0 or 1;
—X comprises a functional group capable of connecting to a reactive polymer that is amenable to elimination under physiological conditions and said second functional group —Y²;
at least one of —R¹, —R² and —R⁵ comprises said first functional group —Y¹ capable of connecting to a polymer;
one and only one of —R¹ and —R² is selected from the group consisting of —H, alkyl, arylalkyl, and heteroarylalkyl;
optionally, —R¹ and —R² may be joined to form a 3- to 8-membered ring;
at least one or both of —R¹ and —R² is independently selected from the group consisting of —CN, —NO₂, aryl, heteroaryl, alkenyl, alkynyl, —COR³, —SOR³, —SO₂R³ and —SR⁴;
—R³ is selected from the group consisting of —H, alkyl, aryl, arylalkyl, heteroaryl, heteroarylalkyl, —OR⁹ and —NR⁹₂;
—R⁴ is selected from the group consisting of alkyl, aryl, arylalkyl, heteroaryl and heteroarylalkyl;
each —R⁵ is independently selected from the group consisting of —H, alkyl, alkenylalkyl, alkynylalkyl, (OCH₂CH₂)ₚO-alkyl with p being an integer ranging from 1 to 1000, aryl, arylalkyl, heteroaryl and heteroarylalkyl;
each —R⁹ is independently selected from the group consisting of —H and alkyl or both —R⁹ together with the nitrogen to which they are attached form a heterocyclic ring; and wherein the moiety of formula (PL-1) is optionally further substituted.

The following paragraphs describe such hydrogel in more detail.

In certain embodiments —X of formula (PL-1) is selected from the group consisting of succinimidyl carbonate, sulfosuccinimidyl carbonate halides, thioethers, esters, nitrophenyl carbonate, chloroformate, fluoroformate, optionally substituted phenols and formula (PL-2)

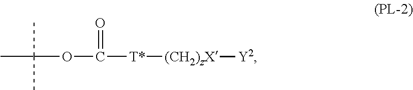

wherein
the dashed line indicates attachment to the remainder of formula (PL-1);
-T*- is selected from the group consisting of —O—, —S— and —NR⁶—;
z is an integer selected from the group consisting of 1, 2, 3, 4, 5 and 6;
—X′— is absent or is selected from the group consisting of —OR⁷— and —SR⁷—;
—Y² is a functional group capable of connecting with a reactive polymer;
—R⁶ is selected from the group consisting of —H, alkyl, aryl, heteroaryl, arylalkyl, and heteroarylalkyl; and
—R⁷ is selected from the group consisting of alkylene, phenylene and (OCH₂CH₂)ₚ, with p being an integer ranging from 1 to 1000.

In certain embodiments —X of formula (PL-1) comprises an activated carbonate such as succinimidyl carbonate, sulfosuccinimidyl carbonate, or nitrophenyl carbonate. In certain embodiments —X of formula (PL-1) comprises a carbonyl halide such as O(C═O)Cl or O(C═O)F. In certain embodiments —X of formula (PL-1) has the formula (PL-2). In certain embodiments —X of formula (PL-1) is OR⁷ or SR⁷, wherein R⁷ is optionally substituted alkylene, optionally substituted phenylene or (OCH₂CH₂)ₚ, wherein p is 1 to 1000.

In certain embodiments p of formula (PL-2) is an integer ranging from 1 to 100. In certain embodiments p of formula (PL-2) is an integer ranging from 1 to 10.

In certain embodiments —Y¹ of formula (PL-1) and —Y² of formula (PL-2) independently comprise N₃, NH₂, NH—CO₂^{t}Bu, SH, S^{t}Bu, maleimide, CO₂H, CO₂^{t}Bu, 1,3-diene, cyclopentadiene, furan, alkyne, cyclooctyne, acrylate or acrylamide, wherein ^{t}Bu is tert-butyl, and wherein when one of —Y¹ or —Y² comprises N₃ the other does not comprise alkyne or cyclooctyne; when one of —Y¹ or —Y² comprises SH the other does not comprise maleimide, acrylate or acrylamide; when one of —Y¹ or —Y² comprises NH₂ the other does not comprise CO₂H; when one of —Y¹ or —Y² comprises 1,3-diene or cyclopentadiene the other does not comprise furan.

In certain embodiments the cleavable crosslinker compound is of formula (PL-3)

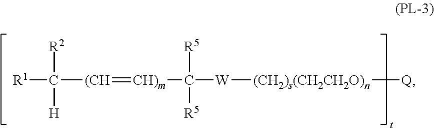

wherein
m is 0 or 1;
n is an integer selected from 1 to 1000;
s is 0, 1 or 2;
t is selected from the group consisting of 2, 4, 8, 16 and 32;
—W— is selected from the group consisting of —O(C═O)O—, —O(C═O)NH—, —O(C═O)S—, —O(C═O)NR⁶CH₂O— and —O(C═O)NR⁶S—;
-Q is a core group having a valency=t; which connects the multiple arms of the cleavable crosslinking compound,
wherein t is an integer selected from 2, 4, 8, 16 and 32, and
wherein —R¹, —R² and —R⁵ are defined as in formula (PL-1).

In certain embodiments t of formula (PL-3) is 2. In certain embodiments t of formula (PL-3) is 4. In certain embodiments t of formula (PL-3) is 8. In certain embodiments t of formula (PL-3) is 16. In certain embodiments t of formula (PL-3) is 32.

In certain embodiments -Q of formula (PL-3) has a structure selected from the group consisting of

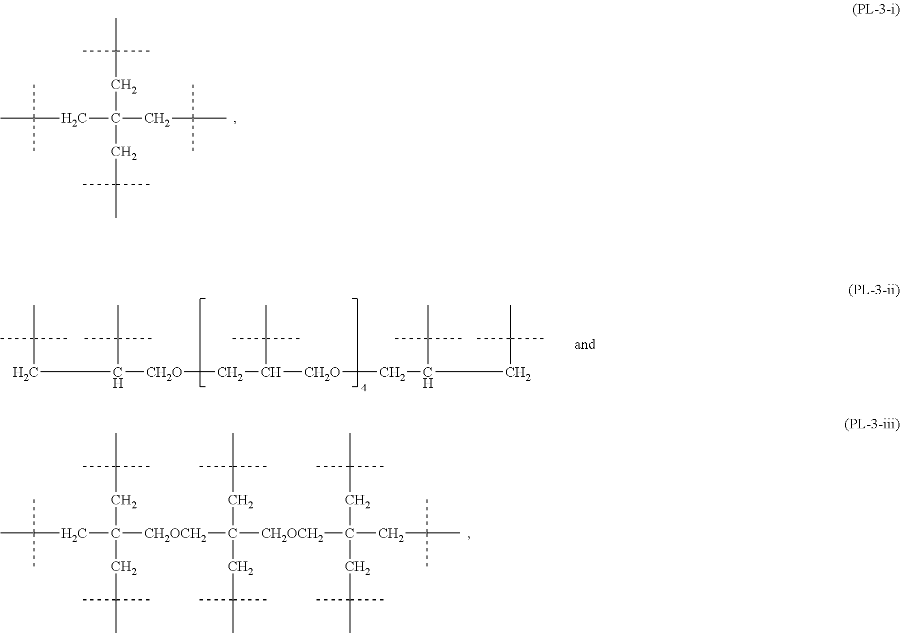

wherein the dashed lines indicate attachment to the remainder of the cleavable crosslinker compound.

In certain embodiments -Q of formula (PL-3) has the structure of (PL-3-i). In certain embodiments -Q of formula (PL-3) has the structure of (PL-3-ii). In certain embodiments -Q of formula (PL-3) has the structure of (PL-3-iii).

In certain embodiments the cleavable crosslinker compound is of formula (PL-3), wherein m is 0, n is approximately 100, s is 0, t is 4, —W— is —O(C═O)NH—, -Q has the structure of (PL-3i), —R² is H, one —R⁵ is —H and the other —R⁵ is (CH₂)₅N₃, and —R¹ is (4-chlorophenyl)SO₂, phenyl substituted with —SO₂, morpholino-SO₂, or —CN.

In certain embodiments —Y¹ of formula (PL-3) comprises N₃, NH₂, NH—CO₂^{t}Bu, SH, S^{t}Bu, maleimide, CO₂H, CO₂^{t}Bu, 1,3-diene, cyclopentadiene, furan, alkyne, cyclooctyne, acrylate or acrylamide, wherein ^{t}Bu is tert-butyl.

In certain embodiments each —Y¹ of formula (PL-1) or (PL-3) and —Y² of formula (PL-2) independently comprises N₃, NH₂, NH—CO₂^{t}Bu, SH, S^{t}Bu, maleimide, CO₂H, CO₂^{t}Bu, 1,3-diene, cyclopentadiene, furan, alkyne, cyclooctyne, acrylate or acrylamide.

In certain embodiments one of —Y¹ and —Y² is azide and the other is a reactive functional group selected from the group consisting of acetylene, cyclooctyne, and maleimide. In certain embodiments one of —Y¹ and —Y² is thiol and the other is a reactive functional group selected from the group consisting of maleimide, acrylate, acrylamide, vinylsulfone, vinylsulfonamide, and halocarbonyl. In certain embodiments one of —Y¹ and —Y² is amine and the other is a selective reactive functional group selected from carboxylic acid and activated carboxylic acid. In certain embodiments one of —Y¹ and —Y² is maleimide and the other is a selective reactive functional group selected from the group consisting of 1,3-diene, cyclopentadiene, and furan.

In certain embodiments the first and any second polymer is selected from the group consisting of homopolymeric or copolymeric polyethylene glycols, polypropylene glycols, poly(N-vinylpyrrolidone), polymethacrylates, polyphosphazenes, polylactides, polyacrylamides, polyglycolates, polyethylene imines, agaroses, dextrans, gelatins, collagens, polylysines, chitosans, alginates, hyaluronans, pectins and carrageenans that either comprise suitable reactive functionalities or is of formula [Y³—(CH₂)ₛ(CH₂CH₂O)ₙ]ₜQ, wherein —Y³ is a reactive functional group, s is 0, 1 or 2, n is an integer selected from the group ranging from 10 to 1000, -Q is a core group having valency t, and t is an integer selected from the group consisting of 2, 4, 8, 16 and 32.

In certain embodiments the first polymer comprises a multi-arm polymer. In certain embodiments the first polymer comprises at least three arms. In certain embodiments the first polymer comprises at least four arms. In certain embodiments the first polymer comprises at least five arms. In certain embodiments the first polymer comprises at least six arms. In certain embodiments the first polymer comprises at least seven arms. In certain embodiments the first polymer comprises at least eight arms.

In certain embodiments the second polymer comprises a multi-arm polymer. In certain embodiments the second polymer comprises at least three arms. In certain embodiments the second polymer comprises at least four arms. In certain embodiments the second polymer comprises at least five arms. In certain embodiments the second polymer comprises at least six arms. In certain embodiments the second polymer comprises at least seven arms. In certain embodiments the second polymer comprises at least eight arms.

In certain embodiments the first polymer comprises a 2-arm polyethylene glycol polymer. In certain embodiments the first polymer comprises a 4-arm polyethylene glycol polymer. In certain embodiments the first polymer comprises an 8-arm polyethylene glycol polymer. In certain embodiments the first polymer comprises a 16-arm polyethylene glycol polymer. In certain embodiments the first polymer comprises a 32-arm polyethylene glycol polymer.

In certain embodiments the second polymer comprises a 2-arm polyethylene glycol polymer. In certain embodiments the second polymer comprises a 4-arm polyethylene glycol polymer. In certain embodiments the second polymer comprises an 8-arm polyethylene glycol polymer. In certain embodiments the second polymer comprises a 16-arm polyethylene glycol polymer. In certain embodiments the second polymer comprises a 32-arm polyethylene glycol polymer.

In certain embodiments the first and a second reactive polymer are reacted with said cleavable crosslinker compound, either sequentially or simultaneously.

In certain embodiments the first and second functional groups are the same.

Only in the context of formulas (PL-1), (PL-2) and (PL-3) the terms used have the following meaning:

The term “a moiety capable of being cleaved by elimination under physiological conditions” refers to a structure comprising a group H—C—(CH═CH)ₘ—C—X′ wherein m is 0 or 1 and X′ is a leaving group, wherein an elimination reaction as described above to remove the elements of HX′ can occur at a rate such that the half-life of the reaction is between 1 and 10,000 hours under physiological conditions of pH and temperature. Preferably, the half-life of the reaction is between 1 and 5,000 hours, and more preferably between 1 and 1,000 hours, under physiological conditions of pH and temperature. By physiological conditions of pH and temperature is meant a pH of between 7 and 8 and a temperature between 30 and 40 degrees centigrade

The term “reactive polymer and reactive oligomer” refers to a polymer or oligomer comprising functional groups that are reactive towards other functional groups, most preferably under mild conditions compatible with the stability requirements of peptides, proteins, and other biomolecules. Suitable functional groups found in reactive polymers include maleimides, thiols or protected thiols, alcohols, acrylates, acrylamides, amines or protected amines, carboxylic acids or protected carboxylic acids, azides, alkynes including cycloalkynes, 1,3-dienes including cyclopentadienes and furans, alpha-halocarbonyls, and N-hydroxysuccinimidyl, N-hydroxysulfosuccinimidyl, or nitrophenyl esters or carbonates.

The term “functional group capable of connecting to a reactive polymer” refers to a functional group that reacts to a corresponding functional group of a reactive polymer to form a covalent bond to the polymer. Suitable functional groups capable of connecting to a reactive polymer include maleimides, thiols or protected thiols, acrylates, acrylamides, amines or protected amines, carboxylic acids or protected carboxylic acids, azides, alkynes including cycloalkynes, 1,3-dienes including cyclopentadienes and furans, alpha-halocarbonyls, and N-hydroxysuccinimidyl, N-hydroxysulfosuccinimidyl, or nitrophenyl esters or carbonates.

The term “substituted” refers to an alkyl, alkenyl, alkynyl, aryl, or heteroaryl group comprising one or more substituent groups in place of one or more hydrogen atoms. Substituent groups may generally be selected from halogen including F, Cl, Br, and I; lower alkyl including linear, branched, and cyclic; lower haloalkyl including fluoroalkyl, chloroalkyl, bromoalkyl, and iodoalkyl; OH; lower alkoxy including linear, branched, and cyclic; SH; lower alkylthio including linear, branched, and cyclic; amino, alkylamino, dialkylamino, silyl including alkylsilyl, alkoxysilyl, and arylsilyl; nitro; cyano; carbonyl; carboxylic acid, carboxylic ester, carboxylic amide; aminocarbonyl; aminoacyl; carbamate; urea; thiocarbamate; thiourea; ketone; sulfone; sulfonamide; aryl including phenyl, naphthyl, and anthracenyl; heteroaryl including 5-member heteroaryls including as pyrrole, imidazole, furan, thiophene, oxazole, thiazole, isoxazole, isothiazole, thiadiazole, triazole, oxadiazole, and tetrazole, 6-member heteroaryls including pyridine, pyrimidine, pyrazine, and fused heteroaryls including benzofuran, benzothiophene, benzoxazole, benzimidazole, indole, benzothiazole, benzisoxazole, and benzisothiazole.

The properties of R¹ and R² may be modulated by the optional addition of electron-donating or electron-withdrawing substituents. By the term “electron-donating group” is meant a substituent resulting in a decrease in the acidity of the R¹R²CH; electron-donating groups are typically associated with negative Hammett σ or Taft σ* constants and are well-known in the art of physical organic chemistry. (Hammett constants refer to aryl/heteroaryl substituents, Taft constants refer to substituents on non-aromatic moieties.) Examples of suitable electron-donating substituents include lower alkyl, lower alkoxy, lower alkylthio, amino, alkylamino, dialkylamino, and silyl.

The term “electron-withdrawing group” refers to a substituent resulting in an increase in the acidity of the R¹R²CH group; electron-withdrawing groups are typically associated with positive Hammett σ or Taft σ* constants and are well-known in the art of physical organic chemistry. Examples of suitable electron-withdrawing substituents include halogen, difluoromethyl, trifluoromethyl, nitro, cyano, C(═O)—R^{x}, wherein —R^{x} is H, lower alkyl, lower alkoxy, or amino, or S(O)ₘR^{Y}, wherein m is 1 or 2 and —R^{Y} is lower alkyl, aryl, or heteroaryl. As is well-known in the art, the electronic influence of a substituent group may depend upon the position of the substituent. For example, an alkoxy substituent on the ortho- or para-position of an aryl ring is electron-donating, and is characterized by a negative Hammett σ constant, while an alkoxy substituent on the meta-position of an aryl ring is electron-withdrawing and is characterized by a positive Hammett σ constant.

The terms “alkyl”, “alkenyl”, and “alkynyl” include linear, branched or cyclic hydrocarbon groups of 1 to 8 carbons or 1 to 6 carbons or 1 to 4 carbons wherein alkyl is a saturated hydrocarbon, alkenyl includes one or more carbon-carbon double bonds and alkynyl includes one or more carbon-carbon triple bonds. Unless otherwise specified these contain 1 to 6 carbons.

The term “aryl” includes aromatic hydrocarbon groups of 6 to 18 carbons, preferably 6 to 10 carbons, including groups such as phenyl, naphthyl, and anthracenyl. “Heteroaryl” includes aromatic rings comprising 3 to 15 carbons containing at least one N, O or S atom, preferably 3 to 7 carbons containing at least one N, O or S atom, including groups such as pyrrolyl, pyridyl, pyrimidinyl, imidazolyl, oxazolyl, isoxazolyl, thiazolyl, isothiazolyl, quinolyl, indolyl, indenyl, and similar.

The term “halogen” includes fluoro, chloro, bromo and iodo.

The term “maleimido” is a group of the formula

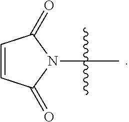 [SMILES: *N1C(=O)C=CC1=O]

In certain embodiments Z is a hydrogel as disclosed in WO2020/206358 A1. In particular, in certain embodiments Z is a hydrogel produced by a method comprising the steps of
(a) providing a first prepolymer comprising a multi-arm polymer —P², wherein said first prepolymer is of formula (PL-4)

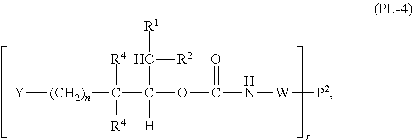

wherein
n is an integer selected from 0, 1, 2, 3, 4, 5 and 6;
r is an integer higher than 2;
—Y is a reactive functional group for connecting said first prepolymer to a second prepolymer;
—R¹ and —R² are independently an electron-withdrawing group, alkyl, or —H, and wherein at least one of —R¹ and —R² is an electron-withdrawing group;
each —R⁴ is independently C₁-C₃ alkyl or the two —R⁴ form together with the carbon atom to which they are attached a 3- to 6-membered ring;
—W— is absent or is

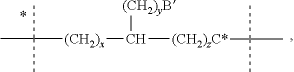

wherein the dashed line marked with the asterisk indicates the attachment to —NH— and the unmarked dashed line indicates the attachment to —P²;
each of x, y, and z is independently an integer selected from 0, 1, 2, 3, 4, 5 and 6;
—B′ is —NH₂, —ONH₂, ketone, aldehyde, —SH, —OH, —CO₂H, carboxamide group, or a group comprising a cyclooctyne or bicyclononyne; and
—C* is carboxamide, thioether, thiosuccinimidyl, triazole, or oxime;
(b) providing the second prepolymer comprising a multi-arm polymer —P¹ wherein each arm is terminated by a reactive functional group —Y″ that reacts with —Y of step (a);
(c) mixing the two prepolymers of steps (a) and (b) under conditions wherein —Y and —Y″ react to form a linkage —Y*—; and optionally
(d) isolating the resulting hydrogel.

Accordingly, —Z is a hydrogel obtainable from the method described above. In certain embodiments the hydrogel produced by the preceding method is degradable.

In certain embodiments —Y and —Y″ react under step (c) to form an insoluble hydrogel matrix comprising crosslinks of formula (PL-4′):

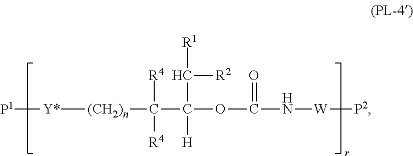

wherein n, r, —P¹, —Y*—, —R⁴, —R¹, —R², —W— and —P² are as defined above.

In certain embodiments n of formula (PL-4) or (PL-4′) is an integer selected from 1, 2, 3, 4, 5 and 6. In certain embodiments n of formula (PL-4) or (PL-4′) is an integer selected from 1, 2 and 3. In certain embodiments n of formula (PL-4) or (PL-4′) is an integer selected from 0, 1, 2 and 3. In certain embodiments n of formula (PL-4) or (PL-4′) is 1. In certain embodiments n of formula (PL-4) is 2. In certain embodiments n of formula (PL-4) or (PL-4′) is 3.

In certain embodiments the multi-arm —P² of formula (PL-4) or (PL-4′) is an r-armed polymer, wherein r is an integer selected from 2, 3, 4, 5, 6, 7, 8, 9, 10, 11 and 12. In certain embodiments r of formula (PL-4) or (PL-4′) is an integer selected from 2, 3, 4, 5, 6, 7 and 8. In certain embodiments r of formula (PL-4) or (PL-4′) is an integer selected from 2, 4, 6 and 8. In certain embodiments r of formula (PL-4) or (PL-4′) is 2. In certain embodiments r of formula (PL-4) or (PL-4′) is 4. In certain embodiments r of formula (PL-4) or (PL-4′) is 6. In certain embodiments r of formula (PL-4) or (PL-4′) is 8.

In certain embodiments —P² of formula (PL-4) or (PL-4′) has a molecular weight of at least 1 kDa. In certain embodiments —P² of formula (PL-4) or (PL-4′) has a molecular weight of 1 to 100 kDa. In certain embodiments —P² of formula (PL-4) or (PL-4′) has a molecular weight of 1 to 80 kDa. In certain embodiments —P² of formula (PL-4) or (PL-4′) has a molecular weight of 1 to 60 kDa. In certain embodiments —P² of formula (PL-4) or (PL-4′) has a molecular weight of 1 to 40 kDa. In certain embodiments —P² of formula (PL-4) or (PL-4′) has a molecular weight of 1 to 20 kDa. In certain embodiments —P² of formula (PL-4) or (PL-4′) has a molecular weight of 1 to 10 kDa. In certain embodiments —P² of formula (PL-4) or (PL-4′) has a molecular weight of 1 to 5 kDa. In certain embodiments —P² of formula (PL-4) or (PL-4′) has a molecular weight of about 20 kDa. In certain embodiments —P² of formula (PL-4) or (PL-4′) has a molecular weight of about 40 kDa. In certain embodiments —P² of formula (PL-4) or (PL-4′) has a molecular weight of about 60 kDa. In certain embodiments —P² of formula (PL-4) or (PL-4′) has a molecular weight of about 80 kDa.

In certain embodiments the multi-arm polymer —P¹ of step (b) is an r-armed polymer, wherein r is an integer selected from 2, 3, 4, 5, 6, 7, 8, 9, 10, 11 and 12. In certain embodiments the multi-arm —P¹ of step (b) is an r-armed polymer, wherein r is an integer selected from 2, 3, 4, 5, 6, 7 and 8. In certain embodiments the multi-arm —P¹ of step (b) is an r-armed polymer, wherein r is an integer selected from 2, 4, 6 and 8. In certain embodiments the multi-arm —P¹ of step (b) is an r-armed polymer, wherein r is 2. In certain embodiments the multi-arm —P¹ of step (b) is an r-armed polymer, wherein r is 4. In certain embodiments the multi-arm —P¹ of step (b) is an r-armed polymer, wherein r is 6. In certain embodiments the multi-arm —P¹ of step (b) is an r-armed polymer, wherein r is 8.

In certain embodiments —P¹ of step (b) has a molecular weight of at least 1 kDa. In certain embodiments the multi-arm polymer —P¹ of step (b) has a molecular weight of 1 to 100 kDa. In certain embodiments the multi-arm polymer —P¹ of step (b) has a molecular weight of 1 to 80 kDa. In certain embodiments the multi-arm polymer —P¹ of step (b) has a molecular weight of 1 to 60 kDa. In certain embodiments the multi-arm polymer —P¹ of step (b) has a molecular weight of 1 to 40 kDa. In certain embodiments the multi-arm polymer —P¹ of step (b) has a molecular weight of 1 to 20 kDa. In certain embodiments the multi-arm polymer —P¹ of step (b) has a molecular weight of 1 to 10 kDa. In certain embodiments the multi-arm polymer —P¹ of step (b) has a molecular weight of 1 to 5 kDa. In certain embodiments the multi-arm polymer —P¹ of step (b) has a molecular weight of about 20 kDa. In certain embodiments the multi-arm polymer —P¹ of step (b) has a molecular weight of about 40 kDa. In certain embodiments the multi-arm polymer —P¹ of step (b) has a molecular weight of about 60 kDa. In certain embodiments the multi-arm polymer —P¹ of step (b) has a molecular weight of about 80 kDa.

In certain embodiments —P¹ of step (b) and —P² of formula (PL-4) or (PL-4′) comprise poly(ethylene glycol) (PEG), poly(ethylene oxide) (PEO), poly(ethylene imine) (PEI), dextrans, hyaluronic acids, or co-polymers thereof. In certain embodiments —P¹ of step (b) and P² of formula (PL-4) or (PL-4′) are PEG-based polymers. In certain embodiments —P¹ of step (b) and —P² of formula (PL-4) or (PL-4′) are hyaluronic acid-based polymers.

In certain embodiments —R¹ and —R² of formula (PL-4) or (PL-4′) are independently electron-withdrawing groups, alkyl, or —H, and wherein at least one of —R¹ and —R² is an electron-withdrawing group.

In certain embodiments the electron-withdrawing group of —R¹ and —R² of formula (PL-4) or (PL-4′) is —CN, —NO₂, optionally substituted aryl, optionally substituted heteroaryl, optionally substituted alkenyl, optionally substituted alkynyl, —COR³, —SOR³, or —SO₂R³, wherein —R³ is —H, optionally substituted alkyl, optionally substituted aryl, optionally substituted arylalkyl, optionally substituted heteroaryl, optionally substituted heteroarylalkyl, —OR⁸ or —NR⁸₂, wherein each —R⁸ is independently —H or optionally substituted alkyl, or both —R⁸ groups are taken together with the nitrogen to which they are attached to form a heterocyclic ring; or —SR⁹, wherein —R⁹ is optionally substituted alkyl, optionally substituted aryl, optionally substituted arylalkyl, optionally substituted heteroaryl, or optionally substituted heteroarylalkyl.

In certain embodiments the electron-withdrawing group of —R¹ and —R² of formula (PL-4) or (PL-4′) is —CN. In certain embodiments the electron-withdrawing group of —R¹ and —R² of formula (PL-4) or (PL-4′) is —NO₂. In certain embodiments the electron-withdrawing group of —R¹ and —R² of formula (PL-4) or (PL-4′) is optionally substituted aryl containing 6 to 10 carbons. In certain embodiments the electron-withdrawing group of —R¹ and —R² of formula (PL-4) or (PL-4′) is optionally substituted phenyl, naphthyl, or anthracenyl. In certain embodiments the electron-withdrawing group of —R¹ and —R² of formula (PL-4) or (PL-4′) is optionally substituted heteroaryl comprising 3 to 7 carbons and containing at least one N, O, or S atom. In certain embodiments the electron-withdrawing group of —R¹ and —R² of formula (PL-4) or (PL-4′) is optionally substituted pyrrolyl, pyridyl, pyrimidinyl, imidazolyl, oxazolyl, isoxazolyl, thiazolyl, isothiazolyl, quinolyl, indolyl, or indenyl. In certain embodiments the electron-withdrawing group of —R¹ and —R² of formula (PL-4) or (PL-4′) is optionally substituted alkenyl containing 2 to 20 carbon atoms. In certain embodiments the electron-withdrawing group of —R¹ and —R² of formula (PL-4) or (PL-4′) is optionally substituted alkynyl containing 2 to 20 carbon atoms. In certain embodiments the electron-withdrawing group of —R¹ and —R² of formula (PL-4) or (PL-4′) is —COR³, —SOR³, or —SO₂R³, wherein R³ is —H, optionally substituted alkyl containing 1 to 20 carbon atoms, optionally substituted aryl, optionally substituted arylalkyl, optionally substituted heteroaryl, optionally substituted heteroarylalkyl, —OR⁸ or —NR⁸₂, wherein each —R⁸ is independently —H or optionally substituted alkyl containing 1 to 20 carbon atoms, or both —R⁸ groups are taken together with the nitrogen to which they are attached to form a heterocyclic ring. In certain embodiments the electron-withdrawing group of —R¹ and —R² of formula (PL-4) or (PL-4′) is —SR⁹, wherein —R⁹ is optionally substituted alkyl containing 1 to 20 carbon atoms, optionally substituted aryl, optionally substituted arylalkyl, optionally substituted heteroaryl, or optionally substituted heteroarylalkyl. In certain embodiments at least one of —R¹ and —R² is —CN or —SO₂R³.

In certain embodiments at least one of —R¹ and —R² of formula (PL-4) or (PL-4′) is —CN, —SOR³ or —SO₂R³. In certain embodiments at least one of —R¹ and —R² of formula (PL-4) or (PL-4′) is —CN or —SO₂R³. In certain embodiments at least one of —R¹ and —R² of formula (PL-4) or (PL-4′) is —CN or —SO₂R³, wherein —R³ is optionally substituted alkyl, optionally substituted aryl, or —NR⁸₂. In certain embodiments at least one of —R¹ and —R² of formula (PL-4) or (PL-4′) is —CN, —SO₂N(CH₃)₂, —SO₂CH₃, phenyl substituted with —SO₂, phenyl substituted with —SO₂ and —Cl, —SO₂N(CH₂CH₂)₂O, —SO₂CH(CH₃)₂, —SO₂N(CH₃)(CH₂CH₃), or —SO₂N(CH₂CH₂OCH₃)₂.

In certain embodiments each —R⁴ of formula (PL-4) or (PL-4′) is independently C₁-C₃ alkyl or taken together may form a 3- to 6-membered ring. In certain embodiments each —R⁴ of formula (PL-4) or (PL-4′) is independently C₁-C₃ alkyl. In certain embodiments both —R⁴ of formula (PL-4) or (PL-4′) are methyl.

In certain embodiments —Y and —Y″ are independently selected from the group consisting of amine, aminooxy, ketone, aldehyde, maleimidyl, thiol, alcohol, azide, 1,2,4,6-tetrazinyl, trans-cyclooctenyl, bicyclononynyl, cyclooctynyl, and protected variants thereof.

In certain embodiments Y and Y″ may react with each other such as in a selective way. For example, when —Y is amine, —Y″ is carboxylic acid, active ester, or active carbonate to yield a residual connecting functional group —Y*— that is amide or carbamate. As another example, when —Y is azide, —Y″ is alkynyl, bicyclononynyl, or cyclooctynyl to yield a residual connecting functional group —Y*— that is 1,2,3-triazole. As another example, when —Y is NH₂O, —Y″ is ketone or aldehyde to yield a residual connecting functional group —Y*— that is oxime. As another example, when —Y is SH, —Y″ is maleimide or halocarbonyl to yield a residual connecting functional group —Y*— that is thiosuccinimidyl or thioether. Similarly, these roles of —Y and —Y″ can be reversed to yield —Y*— of opposing orientation.

In certain embodiments —Y*— comprises an amide, oxime, 1,2,3-triazole, thioether, thiosuccinimide, or ether. In certain embodiments —Y*— is -L²-.

These conjugation reactions may be performed under conditions known in the art, for example when —Y is azide and —Y″ is cyclooctyne the conjugation occurs in any solvent wherein both components show adequate solubility, although it is known that aqueous solutions show more favorable reaction rates. When mixed in an appropriate solvent, typically an aqueous buffer at a pH of 2 to 7 when —Y and —Y″ are azide/cyclooctyne, or at a pH of 6 to 9 when —Y and —Y″ are an activated ester and an amine, the —Y and —Y″ groups react to form an insoluble hydrogel matrix comprising crosslinks of formula (PL-4′). This process may be carried out in bulk phase, or under conditions of emulsification in a mixed organic/aqueous system so as to form microparticle suspensions such as microspheres that are suitable for injection.

In certain embodiments a conjugate comprising a hydrogel Z is produced by a method comprising the steps of
(a) providing a first prepolymer of formula (PL-4)
(b) reacting the prepolymer of formula (PL-4) with a linker-drug of formula (PL-5)

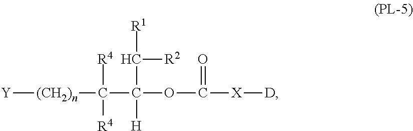

wherein
n, —R¹, —R², —R⁴ and —Y are as defined in formula (PL-4);
D is a drug moiety;
—X— is absent when -D is a drug moiety connected through an amine, or —X— is —N(R⁶)CH₂— when -D is a drug moiety connected through a phenol, alcohol, thiol, thiophenol, imidazole, or non-basic amine; wherein —R⁶ is optionally substituted C₁-C₆ alkyl, optionally substituted aryl, or optionally substituted heteroaryl;
so that —Y of formula (PL-5) reacts with —B′ of formula (PL-4);
(c) providing the second prepolymer comprising a multi-arm polymer —P¹ wherein each arm is terminated by a reactive functional group —Y″ that reacts with —Y of step (a) and wherein embodiments for —P¹ are described above;
(d) mixing the two prepolymers of steps (a) and (b) under conditions wherein —Y and —Y″ react to form a residual connecting functional group —Y*—, and optionally
(e) isolating the resulting hydrogel.

In certain embodiments a conjugate is obtained by a method comprising the step of reacting a hydrogel Z with the linker-drug of formula (PL-5), wherein —B′ on the hydrogel Z reacts with —Y of formula (PL-5).

Only in the context of formulas (PL-4), (PL-4′) and (PL-5) the terms used have the following meaning:

The term “alkyl” refers to linear, branched, or cyclic saturated hydrocarbon groups of 1 to 20, 1 to 12, 1 to 8, 1 to 6, or 1 to 4 carbon atoms. In certain embodiments an alkyl is linear or branched. Examples of linear or branched alkyl groups include methyl, ethyl, n-propyl, isopropyl, n-butyl, t-butyl, isobutyl, sec-butyl, n-pentyl, n-hexyl, n-heptyl, n-octyl, n-nonyl, and n-decyl. In certain embodiments an alkyl is cyclic. Examples of cyclic alkyl groups include cyclopropyl, cyclobutyl, cyclopentyl, cyclopentadienyl, and cyclohexyl.

The term “alkoxy” refers to alkyl groups bonded to oxygen, including methoxy, ethoxy, isopropoxy, cyclopropoxy, and cyclobutoxy.

The term “alkenyl” refers to non-aromatic unsaturated hydrocarbons with carbon-carbon double bonds and 2 to 20, 2 to 12, 2 to 8, 2 to 6, or 2 to 4 carbon atoms.

The term “alkynyl” refers to non-aromatic unsaturated hydrocarbons with carbon-carbon triple bonds and 2 to 20, 2 to 12, 2 to 8, 2 to 6, or 2 to 4 carbon atoms.

The term “aryl” refers to aromatic hydrocarbon groups of 6 to 18 carbons, preferably 6 to 10 carbons, including groups such as phenyl, naphthyl, and anthracenyl. The term “heteroaryl” refers to aromatic rings comprising 3 to 15 carbons comprising at least one N, O or S atom, preferably 3 to 7 carbons comprising at least one N, O or S atom, including groups such as pyrrolyl, pyridyl, pyrimidinyl, imidazolyl, oxazolyl, isoxazolyl, thiazolyl, isothiazolyl, quinolyl, indolyl, and indenyl.

In certain embodiments alkenyl, alkynyl, aryl or heteroaryl moieties may be coupled to the remainder of the molecule through an alkyl linkage. Under those circumstances, the substituent will be referred to as alkenylalkyl, alkynylalkyl, arylalkyl or heteroarylalkyl, indicating that an alkylene moiety is between the alkenyl, alkynyl, aryl or heteroaryl moiety and the molecule to which the alkenyl, alkynyl, aryl or heteroaryl is coupled.

The term “halogen” or “halo” refers to bromo, fluoro, chloro and iodo.

The term “heterocyclic ring” or “heterocyclyl” refers to a 3- to 15-membered aromatic or non-aromatic ring comprising at least one N, O, or S atom. Examples include piperidinyl, piperazinyl, tetrahydropyranyl, pyrrolidine, and tetrahydrofuranyl, as well as the exemplary groups provided for the term “heteroaryl” above. In certain embodiments a heterocyclic ring or heterocyclyl is non-aromatic. In certain embodiments a heterocyclic ring or heterocyclyl is aromatic.

The term “optionally substituted” refers to a group may be unsubstituted or substituted by one or more (e.g., 1, 2, 3, 4 or 5) of the substituents which may be the same or different. Examples of substituents include alkyl, alkenyl, alkynyl, halogen, —CN, —OR^{aa}, —SR^{aa}, —NR^{aa}R^{bb}, —NO₂, —C═NH(OR^{aa}), —C(O)R^{aa}, —OC(O)R^{aa}, —C(O)OR^{aa}, —C(O)NR^{aa}R^{bb}, —OC(O)NR^{aa}R^{bb}, —NR^{aa}C(O)R^{bb}, —NR^{aa}C(O)OR^{bb}, —S(O)R^{aa}, —S(O)₂R^{aa}, —NR^{aa}S(O)R^{bb}, —C(O)NR^{aa}S(O)R^{bb}, —NR^{aa}S(O)₂R^{bb}, —C(O)NR^{aa}S(O)₂R^{bb}, —S(O)NR^{aa}R^{bb}, —S(O)₂NR^{aa}R^{bb}, —P(O)(OR^{aa})(OR^{bb}), heterocyclyl, heteroaryl, or aryl, wherein the alkyl, alkenyl, alkynyl, cycloalkyl, heterocyclyl, heteroaryl, and aryl are each independently optionally substituted by —R^{cc}, wherein —R^{aa} and —R^{bb} are each independently —H, alkyl, alkenyl, alkynyl, heterocyclyl, heteroaryl, or aryl, or —R^{aa} and —R^{bb} are taken together with the nitrogen atom to which they attach to form a heterocyclyl, which is optionally substituted by alkyl, alkenyl, alkynyl, halogen, hydroxyl, alkoxy, or —CN, and wherein: each —R^{cc} is independently alkyl, alkenyl, alkynyl, halogen, heterocyclyl, heteroaryl, aryl, —CN, or —NO₂.

In certain embodiments Z′ is degradable. Irradiating degradable polymer carriers is particularly challenging, because the degradable bonds may be damaged or additional cross-links could be introduced during irradiation. Both reactions would alter the biophysical properties of the polymer and potentially impact its intended performance. In the context of the use for drug delivery purposes, this may result in changes in the safety or efficacy profile, or both. It was now surprisingly found that the degradation half-life after irradiation of such degradable Z′ varies by no more than 20% compared to corresponding non-irradiated Z′.

In certain embodiments Z′ comprises a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(alkylene glycols), such as poly(ethylene glycols) and poly(propylene glycol), poly(ethylene oxides), poly(ethyl phosphates), poly(ethyloxazolines), poly(glycolic acids), poly(hydroxyethyl acrylates), poly(hy droxy ethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids) (PLGA), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

In certain embodiments Z′ comprises PLGA. In certain embodiments Z′ is a hydrogel. In certain embodiments Z′ is a poly(alkylene glycol)-based or hyaluronic acid-based hydrogel. In certain embodiments Z′ is a poly(propylene glycol)-based hydrogel.

In certain embodiments the conjugate or complex is a shaped article, such as a coating, mesh, stent or a microparticle.

Another aspect is a conjugate or complex sterilized by the method of the process of the present invention.

Materials and Methods

The transient daptomycin-linker hydrogel conjugates 1 and 2 and the transient resiquimod-linker hydrogel conjugate 3 for the irradiation experiments were synthesized as described below. Conjugate 3 is suspended in aqueous buffer pH 7.4.

All other materials were commercially available.

Process Methods for Transient Resiquimod-Linker Hydrogel Conjugate 3:

Dosimetry

Radiation doses were determined using photometric measurements of radiochromic thin layer films or by electron paramagnetic resonance measurements of alanine-coated microchips.

Temperature Mapping

In certain embodiments temperatures inside the vials, outside the vials, and/or in the vessels were measured using Type T thermocouples (Omega SSRTC-TT-T-30-72) and captured on a thermometer (Sper Scientific 4-channel thermometer).

Analytical Methods for Transient Daptomycin-Linker Hydrogel Conjugates 1 and 2 and Transient Resiquimod-Linker Hydrogel Conjugate 3

Uplc-Ms Analysis:

Analytical ultra-performance UPLC-MS was performed on an Agilent 1290 Infinity II equipped with a Waters BEH300 C18 column coupled to a Single Quad MS System from Agilent.

HPLC-SEC Analysis:

Analytical size exclusion chromatography was performed on an Agilent 1290 Infinity II equipped with a Sepax Zenix SEC-150 column.

### EXAMPLE 1

Transient Daptomycin-Linker PEG-Hydrogel Conjugate 1

Transient daptomycin-linker PEG-hydrogel conjugate 1 was synthesized as described for compound 9b in WO 2020/064844 A1, example 9. The obtained material had a daptomycin content of 470 mg/g dry material.

### EXAMPLE 2

Transient Daptomycin-Linker HA-Hydrogel Conjugate 2

Transient daptomycin-linker HA-hydrogel conjugate was synthesized as described for compound 5 in WO 2020/064847 A1, example 15. The representative material 2 that was used for all experiments had a daptomycin content of 459 mg/g dry material.

### EXAMPLE 3

X-Ray Irradiation of Transient Daptomycin-Linker Hydrogel Conjugates

Two 11-15 mg samples of transient daptomycin-linker PEG-hydrogel conjugate 1 and two 15-17 mg samples of a representative transient daptomycin-linker HA-hydrogel conjugate (equal to compound 2) were separately filled into 2 mL plastic vials under an argon atmosphere. A set of one sample tube of compound 1 and one sample tube of representative compound 2 was irradiated with X-rays at 78° C. in a bed of dry ice and a total dose of 32.1-33.6 kGy to form sample set A consisting of compound 1a (−78° C. irradiated transient daptomycin-linker PEG-hydrogel conjugate) and compound 2a (−78° C. irradiated transient daptomycin-linker HA-hydrogel conjugate).

A second set of one sample tube of compound 1 and one sample tube of representative compound 2 was irradiated with X-rays at room temperature and a total dose of 32.1-33.6 kGy to form sample set B consisting of compound 1b (r.t. irradiated transient daptomycin-linker PEG-hydrogel conjugate) and compound 2b (r.t. irradiated transient daptomycin-linker HA-hydrogel conjugate).

### EXAMPLE 4

Purity of Released Daptomycin from Irradiated and Non-Irradiated Transient Daptomycin-Linker Hydrogel Conjugates

To investigate the influence of X-rays on the purity of the transiently bound daptomycin in the carriers, a time limited cumulative release of daptomycin from compounds 1, 2, 1a, 2a, 1b and 2b was carried out. For that purpose, the materials were incubated under physiological conditions in concentrations of 4 mg/mL for exactly 42 hours. After this time, samples were taken from the supernatants and analyzed by UPLC-MS at 215 nm. Daptomycin itself is prone to degrade under physiological conditions via different pathways. Therefore, intrinsic degradation of daptomycin in the cumulative release solution to a reduced purity of 88-89% was expected and observed. It was additionally found that the purity of the released daptomycin from all irradiated samples was at least 96% of the purity of the released daptomycin from the respective non-irradiated samples.

| | Purity of daptomycin | | |
|---|---|---|---|
| | in the supernatant after | Relative | Compared to |
| Compound | 42 hours cumulative release | purity | compound |
| 1 | 88.0% | / | / |
| 2 | 88.8% | / | / |
| 1a | 85.6% | 97.3% | 1 |
| 2a | 87.1% | 98.1% | 2 |
| 1b | 84.5% | 96.0% | 1 |
| 2b | 85.3% | 96.1% | 2 |

### EXAMPLE 5

Linker Release Kinetics for Irradiated and Non-Irradiated Transient Daptomycin-Linker Hydrogel Conjugates

The linker kinetics with respect to the daptomycin species release from irradiated and non-irradiated transient daptomycin-linker hydrogel conjugates 1, 2, 1a, 2a, 1b and 2b were investigated by incubation of the materials under physiological conditions. Daptomycin is prone to hydrolytic degradation and some minor different degradation pathways upon aqueous incubation. For determination of the linker kinetics on the carriers, the supernatants of the incubated suspensions were analyzed by UPLC-MS at 215 nm and all daptomycin-related peaks were taken into account for the calculation of the linker kinetics. The linker kinetics did not differ between the irradiated and the respective non-irradiated samples with respect to half-life, release rate and the finally reached plateau. Linker half-lives have been determined to be twelve days for the transient daptomycin-linker PEG-hydrogel conjugates 1, 1a and 1b and ten days for the transient daptomycin-linker HA-conjugates 2, 2a and 2b.

### EXAMPLE 6

Degradation Studies of Irradiated and Non-Irradiated Transient Daptomycin-Linker Hydrogel Conjugates

Irradiated and non-irradiated transient daptomycin-linker hydrogel conjugates 1, 2, 1a, 2a, 1b and 2b were analyzed regarding carrier degradation. For that purpose, the materials were incubated under physiological conditions. The samples were visually checked for the presence of the solid carrier particles daily. As soon as no particles could be detected in the sample anymore, the material was deemed to be fully degraded to soluble products. It was found that the degradation time for the HA-hydrogel conjugates 2, 2a and 2b was 52-56 days and did not differ significantly between the non-irradiated and the irradiated samples. The degradation of the irradiated PEG-hydrogel samples 1a and 1b occurred with 27-29 days slightly faster than it was observed for the non-irradiated sample 1 with 40 days. A slight prolongation of the degradation time could be observed for the samples 1b and 2b, which were irradiated at r.t. compared to the samples 1a and 2a, which were treated at −78° C.

| Compound | 1 | 1a | 1b | 2 | 2a | 2b |
|---|---|---|---|---|---|---|
| Irradiation | not | −78° C. | r.t. | not | −78° C. | r.t. |
| temperature | irradiated | | | irradiated | | |
| Time for full | 40 d | 27 d | 29 d | 55 d | 52 d | 56 d |
| degradation | | | | | | |

### EXAMPLE 7

Transient Resiquimod-Linker PEG-Hydrogel Conjugate 3

Transient resiquimod-linker PEG-hydrogel conjugate 3 was synthesized as described for compound 14 in PCT/EP2020/050093, example 8. The obtained material had a resiquimod content of 17.2 mg/g dry material. It was formulated to 0.5 mg resiquimod/mL in phosphate-buffered saline containing 137 mM sodium chloride, 2.68 mM potassium chloride, 4.3 mM sodium phosphate, dibasic, and 1.4 mM potassium phosphate, monobasic, pH 7.4.

### EXAMPLE 8

X-Ray Irradiation of Transient Resiquimod-Linker Hydrogel Conjugates

0.5-mg samples (1-mL fill) of transient resiquimod-linker PEG-hydrogel conjugate 3 are separately filled into 5-mL glass vials under atmospheric gas. Vials with 1.0-mL fill of only aqueous buffer were separately filled into 5-mL glass vials under atmospheric gas.

A set of multiple sample vials of compound 3 are irradiated with x-rays in a vessel containing dry ice, a total absorbed dose of 25-40 kGy with a temperature inside the vials of −50° C.±10° C. during irradiation to form a sample set of compound 3a.

### EXAMPLE 9

E-Beam Irradiation of Transient Resiquimod-Linker Hydrogel Conjugates

0.5-mg samples (1-mL fill) of transient resiquimod-linker PEG-hydrogel conjugate 3 were separately filled into 5-mL glass vials under atmospheric gas. Vials with 1.0-mL fill of only aqueous buffer were separately filled into 5-mL glass vials under atmospheric gas.

A set of multiple sample vials of compound 3 were irradiated with electron beam in a vessel containing dry ice and a target absorbed dose of 25-40 kGy with an executed total absorbed dose of 29-32 kGy to form a sample set of compound 3b. Temperature mapping data for e-beam irradiation demonstrated the temperatures inside vials were colder than −25° C.±10° C.

### EXAMPLE 10

Drug Purity for Irradiated and Non-Irradiated Resiquimod-Linker Hydrogel Conjugates

Irradiated and non-irradiated resiquimod-linker hydrogel conjugates 3 and 3b were tested for resiquimod purity by releasing all or nearly all transiently bound resiquimod from the hydrogel under basic conditions. The samples were incubated under basic conditions overnight, after which they were pH adjusted and the supernatants were analyzed by UPLC-MS at 320 nm. The measured purity values of conjugates 3 and 3b were 99.7% and 98.1%, respectively.

### EXAMPLE 11

Linker Release Kinetics for Irradiated and Non-Irradiated Resiquimod-Linker Hydrogel Conjugates

The resiquimod release rate under physiological conditions of irradiated and non-irradiated resiquimod-linker hydrogel conjugates 3 and 3b was measured. The supernatants of incubated samples were analyzed by UPLC-MS at 320 nm; as resiquimod is stable to the incubation conditions, only the resiquimod peak was considered for the release kinetics determination. The rate of resiquimod release was fit using a first order exponential equation to estimate the half-life of drug release. The resulting half-life parameter fits for conjugates 3 and 3b were 18.1 and 20.8 days, respectively.

### EXAMPLE 12

Accelerated Degradation Studies of Irradiated and Non-Irradiated Resiquimod-Linker Hydrogel Conjugates

The carrier degradation profile under accelerated conditions of irradiated and non-irradiated resiquimod-linker hydrogel conjugates 3 and 3b were measured. Samples were incubated under basic conditions and the supernatants were sampled over the course of one week. After the final timepoint was collected, the supernatant samples were further hydrolyzed overnight to ensure that all transiently bound resiquimod was released from solubilized carrier fragments. The samples were pH adjusted and analyzed by SEC to measure soluble carrier fragments. The degradation rate of the carrier was fit using a five parameter asymmetric sigmoidal equation, which was used to estimate the time to 50% degradation (T50). The T50 values for conjugates 3 and 3b were 73.7 and 75.1 hours, respectively.

### Abbreviations

HA Hyaluronic Acid
HPLC High Performance Liquid Chromatography
PEG Poly(ethylene glycol)
r.t. Room Temperature
SEC Size Exclusion Chromatography
T50 Time to 50% hydrogel degradation
TFA Trifluoroacetic Acid
UPLC-MS Mass Spectrometry Coupled Ultra Performance Liquid Chromatography

1. A process for the irradiation of a water-insoluble conjugate comprising a polymer Z to which a plurality of moieties -L²-L¹-D- is covalently conjugated or of a water-insoluble complex comprising a plurality of releasably and non-covalently bound drug molecules D-H or D-OH embedded in a polymer Z′, wherein the process comprises the steps of
(a) providing said conjugate or complex; and
(b) exposing the conjugate or complex to ionizing radiation;
wherein
each -L²- is independently a chemical bond or is a spacer moiety;
each -L¹- is independently a linker moiety covalently and reversibly attached to -D; and
each -D is independently a drug moiety.

2. The process of claim 1, wherein the process is performed with the water-insoluble conjugate.

3. The process of claim 1, wherein the process is performed with the water-insoluble complex.

4. The process of claim 1, wherein the ionizing radiation is selected from the group consisting of electron beam radiation, X-ray radiation, gamma radiation, proton beam radiation, neutron beam radiation, positron beam radiation, alpha particle radiation, UV radiation and any combination thereof.

5. The process of claim 1, wherein the ionizing radiation is electron beam.

6. The process of claim 1, wherein the ionizing radiation is X-ray radiation.

7. The process of claim 1, wherein the ionizing radiation is gamma radiation.

8. The process of claim 1, wherein the ionizing radiation is proton beam radiation.

9. The process of claim 1, wherein the ionizing radiation is neutron beam radiation.

10. The process of claim 1, wherein the ionizing radiation is positron beam radiation.

11. The process of claim 1, wherein the ionizing radiation is alpha particle radiation.

12. The process of claim 1, wherein the ionizing radiation is UV radiation.

13. The process of claim 1, wherein the ionizing radiation is a combination of one or more selected from the group consisting of electron beam radiation, X-ray radiation, gamma radiation, proton beam radiation, neutron beam radiation, positron beam radiation, alpha particle radiation and UV radiation.

14. The process of claim 1, wherein irradiation is performed as a continuous irradiation or as multiple irradiation exposures.

15. The process of claim 1, wherein the exposing to ionizing radiation in step (b) is performed with a total absorbed radiation dose ranging from 10 to 80 kGy.

16. The process of claim 1, wherein the exposing to ionizing radiation in step (b) is performed with a total absorbed radiation dose ranging from 17.5 to 35 kGy.

17. The process of claim 1, wherein the conjugate or complex is in a liquid formulation in step (b).

18. The process of claim 1, wherein the conjugate or complex is in a dry formulation in step (b).

19. The process of claim 1, wherein step (b) is performed at a temperature ranging from −196° C. to +45° C.

20. The process of claim 1, wherein each -D is independently selected from the group consisting of small molecule drug moieties, large molecule drug moieties, oligonucleotide moieties, peptide nucleic acid moieties, peptide moieties and protein moieties.

21. The process of claim 1, wherein all moieties -D of the conjugate or complex are identical.

22. The process of claim 1, wherein the conjugate or complex comprises more than one type of -D.

23. The process of claim 1, wherein the conjugate or complex comprises two, three, four or five different types of -D.

24. An irradiated water-insoluble conjugate comprising a polymer Z to which a plurality of moieties -L²-L¹-D is covalently and reversibly conjugated or an irradiated water-insoluble complex comprising a plurality of releasably and non-covalently bound drug molecules D-H or D-OH embedded in a polymer Z′ obtainable from the process of claim 1.
